(19) United States
(12) Patent Application Publication
(10) Pub. No.: US 2020/0123265 A1
(43) Pub. Date: Apr. 23, 2020

(54) ANTI-GITR ANTIBODIES AND METHODS OF USE THEREOF

(71) Applicants: Agenus Inc., Lexington, MA (US); Memorial Sloan-Kettering Cancer Center, New York, NY (US); Ludwig Institute for Cancer Research Ltd., Zurich (CH)

(72) Inventors: Nicholas S. WILSON, Somerville, MA (US); Jeremy D. WAIGHT, Everett, MA (US); Gerd RITTER, New York, NY (US); Takemasa TSUJI, Williamsville, NY (US); Olivier LÉGER, Saint-Sixt (FR); Volker SEIBERT, Lorrach (DE); David SCHAER, Mamaroneck, NY (US); Taha MERGHOUB, Jersey City, NJ (US); Dennis J. UNDERWOOD, Jamaica Plain, MA (US); Ana M. GONZALEZ, Cambridge, MA (US); Marc VAN DIJK, Bilthoven (NL)

(21) Appl. No.: 15/781,051

(22) PCT Filed: Dec. 2, 2016
(86) PCT No.: PCT/US2016/064657

§ 371 Date: Jun. 1, 2018

Related U.S. Application Data
(60) Provisional application No. 62/328,542, filed on Apr. 27, 2016.
(60) Provisional application No. 62/262,376, filed on Dec. 2, 2015.

(51) Int. Cl. C07K 16/28

(52) U.S. Cl. CPC C07K 16/2878; C07K 2317/21; C07K 2317/24; A61K 45/06; C07K 2317/34; C07K 2317/56; C07K 2317/76; C07K 2317/33

(57) ABSTRACT

The present disclosure provides antibodies that specifically bind to human GITR, as well as compositions comprising such antibodies. In a specific aspect, the antibodies specifically bind to human GITR and deactivate, reduce, or inhibit GITR activity. The present disclosure also provides methods for treating autoimmune or inflammatory diseases disorders, by administering an antibody that specifically binds to human GITR and deactivates, reduces, or inhibits GITR activity.

### 1. RELATED APPLICATIONS

The instant application claims priority to U.S. Provisional Application Nos. 62/262,376, filed on Dec. 2, 2015, and 62/328,542, filed on Apr. 27, 2016, the disclosures of which are herein incorporated by reference in their entireties.

### 2. SEQUENCE LISTING

The instant application contains a sequence listing which has been submitted electronically in ASCII format and is hereby incorporated by reference in its entirety (said ASCII copy, created on Dec. 1, 2016, is named 3617_018PC03_SeqListing.txt and is 117,074 bytes in size).

### 3. FIELD

The present disclosure relates to antibodies that specifically bind to human glucocorticoid-induced TNF receptor family-related protein (“GITR”), compositions comprising such antibodies, and methods of producing and using those antibodies.

### 4. BACKGROUND

GITR, a member of the tumor necrosis factor receptor superfamily, is an important stimulator of the immune response. Also known as activation-inducible TNFR family receptor (AITR), GITR-D, CD357, and tumor necrosis factor receptor superfamily member 18 (TNFRSF18)), GITR is expressed in many components of the innate and adaptive immune system and stimulates both acquired and innate immunity (Nocentini, G et al., PNAS 94: 6216-6221 (1994); Hanabuchi, S et al., Blood 107:3617-3623 (2006); Nocentini, G & Riccardi, C, Eur J Immunol 35: 1016-1022 (2005); Nocentini, G et al., (2007), Eur J Immunol 37:1165-1169). GITR is expressed in several cells and tissues, including T, B, dendritic (DC), and Natural Killer (NK) cells, and is activated by its ligand, GITRL, mainly expressed on Antigen Presenting Cells (APCs), on endothelial cells, and also in tumor cells.

The GITR/GITRL system participates in the development of autoimmune/inflammatory responses and potentiates response to infection and tumors. For example, treating animals with GITR-Fc fusion protein ameliorates autoimmune/inflammatory diseases, while GITR triggering is effective in treating viral, bacterial, and parasitic infections, as well as in boosting immune response against tumors (Nocentini, G et al., Br J Pharmacol 165: 2089-2099 (2012)). These effects are due to several concurrent mechanisms including: co-activation of effector T-cells, inhibition of regulatory T (Treg) cells, NK-cell co-activation, activation of macrophages, modulation of dendritic cell function, and regulation of the extravasation process. The membrane expression of GITR is increased following T cell activation (Hanabuchi, S et al, (2006), supra; Nocentini, G & Riccardi, C (2005), supra)). Its triggering coactivates effector T lymphocytes (McHugh, R S et al., Immunity 16:311-323 (2002); Shimizu, J et al., Nat Immunol 3:135-142 (2002); Roncheti, S et al., Eur J Immunol 34:613-622 (2004); Tone, M et al., PNAS 100:15059-15064 (2003)). GITR activation increases resistance to tumors and viral infections, is involved in autoimmune/inflammatory processes and regulates leukocyte extravasation (Nocentini, G & Riccardi, C (2005), supra; Cuzzocrea, S et al., J Leukoc Biol. 76:933-940 (2004); Shevach, E M & Stephens, G L, Nat Rev Immunol 6:613-618 (2006); Cuzzocrea, S et al., J Immunol 177:631-641 (2006); Cuzzocrea, S et al., FASEB J 21:117-129 (2007)).

Human GITR is expressed at very low levels in peripheral (non-activated) T cells. After T cell activation, GITR is strongly up-regulated for several days in both CD4⁺ and CD8⁺ cells (Kwon, B et al., J Biol Chem 274:6056-6061 (1999); Gurney, A L et al., Curr Biol 9:215-218 (1999); Ronchetti, S et al. (2004), supra; Shimizu, J et al. (2002) supra; Ji, H B et al. (2004), supra; Ronchetti, S et al., Blood 100:350-352 (2002); Li, Z et al. J Autoimmune 21:83-92 (2003)), with CD4⁺ cells having a higher GITR expression than CD8⁺ cells (Kober, J et al., Eur J Immunol 38:2678-88 (2008); Bianchini, R et al., Eur J Immunol 41:2269-78 (2011)).

As activating GITR results in an enhanced immune response, antibodies that specifically bind to GITR and deactivate, reduce, or inhibit such activation (e.g., antagonist antibodies) are provided herein, e.g., to treat autoimmune disorders and inflammatory diseases.

### 5. SUMMARY

In one aspect, provided herein are antagonist antibodies that specifically bind to GITR (e.g., human GITR).

In one aspect, an isolated antibody that specifically binds to human GITR comprises: (a) a first antigen-binding domain that specifically binds to human GITR; and (b) a second antigen-binding domain that does not specifically bind to an antigen expressed by a human immune cell.

In one aspect, the antigen-binding domain that specifically binds to human GITR comprises: (a) a first heavy chain variable domain (VH) comprising a VH-complementarity determining region (CDR) 1 comprising the amino acid sequence of X₁YX₂MX₃ (SEQ NO:87), wherein X₁ is D, E or G; X₂ is A or V, and X₃ is Y or H; a VH-CDR2 comprising the amino acid sequence of X₁IX₂TX₃SGX₄X₅X₆YNQKFX₇X₈(SEQ ID NO:88), wherein X₁ is V or L, X₂ is R, K or Q, X₃ is Y or F, X₄ is D, E or G, X₅ is V or L, X₆ is T or S, X₇ is K, R or Q, and X₈ is D, E or G; and a VH-CDR3 comprising the amino acid sequence of SGTVRGFAY (SEQ ID NO:3); and (b) a first light chain variable domain (VL) comprising a VL-CDR1 comprising the amino acid sequence of KSSQSLLNSX₁NQKNYLX₂ (SEQ ID NO:90), wherein X₁ is G or S, and X₂ is T or S; a VL-CDR2 comprising the amino acid sequence of WASTRES (SEQ ID NO:5); and a VL-CDR3 comprising the amino acid sequence of QNX₁YSX₂PYT (SEQ ID NO:92), wherein X₁ is D or E; and X₂ is Y, F or S.

In one aspect, the antigen-binding domain that specifically binds to GITR binds to the same epitope of human GITR as an antibody comprising a VH comprising the amino acid sequence of SEQ ID NO:18 and a VL comprising the amino acid sequence of SEQ ID NO:19.

In one aspect, the antigen-binding domain that specifically binds to human GITR exhibits, as compared to binding to a human GITR sequence of residues 26 to 241 of SEQ ID NO:41, reduced or absent binding to a protein identical to residues 26 to 241 of SEQ ID NO:41 except for the presence of a D60A or G63A amino acid substitution, numbered according to SEQ ID NO:41.

In one aspect, the antigen-binding domain that specifically binds to human GITR comprises CDRs comprising the amino acid sequences of SEQ ID NOs: 1-6.

In one aspect, the antigen-binding domain that specifically binds to human GITR comprises a VH and a VL, wherein the VH comprises an amino acid sequence selected from the group consisting of SEQ ID NOs: 18, 20, 22, 24, and 25. In one aspect, the antigen-binding domain that specifically binds to human GITR comprises a VH and a VL, wherein the VL comprises an amino acid sequence selected from the group consisting of SEQ ID NOs: 19, 21, 23, and 26.

In one aspect, the second antigen-binding domain specifically binds to a non-human antigen. In one aspect, the second antigen-binding domain specifically binds to a viral antigen. In one aspect, the viral antigen is an HIV antigen. In one aspect, the second antigen-binding domain specifically binds to chicken albumin or hen egg lysozyme.

In one aspect, the antigen-binding domain that specifically binds to human GITR specifically binds to an epitope of GITR comprising at least one amino acid in residues 60-63 of SEQ ID NO:41. In one aspect, the antigen-binding domain that specifically binds to human GITR specifically binds to each of i) human GITR, comprising amino acid residues 26 to 241 of SEQ ID NO:41; and ii) a variant of cynomolgus GITR, said variant comprising amino acid residues 26-234 of SEQ ID NO:46, wherein the antigen-binding domain that specifically binds to human GITR does not specifically bind to cynomolgus GITR comprising amino acid residues 26-234 of SEQ ID NO:44.

In one aspect, an isolated antibody that specifically binds to human GITR comprises: (a) an antigen-binding domain that specifically binds to human GITR, comprising a first heavy chain and a light chain; and (b) a second heavy chain or a fragment thereof.

In one aspect, the antigen-binding domain that specifically binds to human GITR comprises: (a) a first heavy chain variable domain (VH) comprising a VH complementarity determining region (CDR) 1 comprising the amino acid sequence of X₁YX₂MX₃ (SEQ ID NO:87), wherein X₁ is D, E or G; X₂ is A or V, and X₃ is Y or H; a VH-CDR2 comprising the amino acid sequence of X₁IX₂TX₃SGX₄X₅X₆YNQKFX₇X₈ (SEQ ID NO:88), wherein X₁ is V or L, X₂ is R, K or Q, X₃ S Y or F, X₄ is D, E or G, X₅ is V or L, X₆ is T or S, X₇ is K, R or Q, and X₈ is D, E or G; and a VH-CDR3 comprising the amino acid sequence of SGTVRGFAY (SEQ ID NO:3); and (b) a first light chain variable domain (VL) comprising a VL-CDR1 comprising the amino acid sequence of KSSQSLLNSX₁NQKNYLX₂(SEQ ID NO:90), wherein X₁ is G or 5, and X₂ is T or S; a VL-CDR2 comprising the amino acid sequence of WASTRES (SEQ ID NO:5); and a VL-CDR3 comprising the amino acid sequence of QNX₁YSX₂PYT (SEQ ID NO:92), wherein X₁ is D or E; and X₂ is Y, F or S.

In one aspect, the antigen-binding domain that specifically binds to human GITR comprises CDRs comprising the amino acid sequences of SEQ ID NOs: 1-6.

In one aspect, the antigen-binding domain that specifically binds to human GITR specifically binds to the same epitope of GITR as an antibody comprising a VH comprising the amino acid sequence of SEQ ID NO:18 and a VL comprising the amino acid sequence of SEQ ID NO:19.

In one aspect, the antigen-binding domain that specifically binds to human GITR exhibits, as compared to binding to a human GITR sequence of residues 26 to 241 of SEQ ID NO:41, reduced or absent binding to a protein identical to residues 26 to 241 of SEQ ID NO:41 except for the presence of a D60A or G63A amino acid substitution, numbered according to SEQ ID NO:41.

In one aspect, the antigen-binding domain that specifically binds to human GITR comprises a VH and a VL, wherein the VH comprises an amino acid sequence selected from the group consisting of SEQ ID NOs: 18, 20, 22, 24, and 25. In one aspect, the antigen-binding domain that specifically binds to human GITR comprises a VH and a VL, wherein the VL comprises an amino acid sequence selected from the group consisting of SEQ ID NOs: 19, 21, 23, and 26.

In one aspect, the fragment of the second heavy chain is an Fc fragment.

In one aspect, the antigen-binding domain that specifically binds to human GITR comprises a VH-CDR1, comprising an amino acid sequence selected from the group consisting of SEQ ID NOs: 7-9. In one aspect, the antigen-binding domain that specifically binds to human GITR comprises a VH-CDR2 comprising an amino acid sequence selected from the group consisting of SEQ ID NOs: 10-13. In one aspect, the antigen-binding domain that specifically binds to human GITR comprises a VL-CDR1 comprising the amino acid sequence of SEQ ID NO: 14 or 15. In one aspect, the antigen-binding domain that specifically binds to human GITR comprises a VL-CDR3 comprising the amino acid sequence of SEQ ID NO: 16 or 17. In one aspect, the antigen-binding domain that specifically binds to human GITR comprises VH-CDR1, VH-CDR2, and VH-CDR3 sequences set forth in SEQ ID NOs: 7, 10, and 3; SEQ ID NOs: 8, 11, and 3; SEQ ID NOs: 9, 12, and 3; or SEQ ID NOs: 9, 13, and 3, respectively; and/or VL-CDR1, VL-CDR2, and VL-CDR3 sequences set forth in SEQ ID NOs: 14, 5, and 16; or SEQ ID NOs: 15, 5, and 17, respectively. In one aspect, the antigen-binding domain that specifically binds to human GITR comprises VH-CDR1, VH-CDR2, VH-CDR3, VL-CDR1, VL-CDR2, and VL-CDR3 sequences set forth in SEQ ID NOs: 7, 10, 3, 14, 5, and 16, respectively.

In one aspect, the antigen-binding domain that specifically binds to human GITR comprises a VH comprising the sequence set forth in SEQ ID NO:25, In one aspect, the antigen-binding domain that specifically binds to human GITR comprises a VH comprising an amino acid sequence at least 75%, 80%, 85%, 90%, 95%, or 99% identical to an amino acid sequence selected from the group consisting of SEQ ID NOs: 18, 20, and 24. In one aspect, the antigen-binding domain that specifically binds to human GITR comprises a VH comprising an amino acid sequence selected from the group consisting of SEQ ID NOs: 18, 20, 22, and 24. In one aspect, the antigen-binding domain that specifically binds to human GITR comprises a VH comprising the amino acid sequence of SEQ ID NO:18.

In one aspect, the antigen-binding domain that specifically binds to human GITR comprises a heavy chain comprising the amino acid sequence of SEQ ID NOs: 29, 30, or 36. In one aspect, the antigen-binding domain that specifically binds to human GITR comprises a heavy chain comprising the amino acid sequence of SEQ ID NOs: 74, 75, or 81

In one aspect, the antigen-binding domain that specifically binds to human GITR comprises a VH comprising an amino acid sequence derived from a human IGHV1-2 germline sequence.

In one aspect, the antigen-binding domain that specifically binds to human GITR comprises a VL comprising the amino acid sequence of SEQ ID NO: 26. In one aspect, the antigen-binding domain that specifically binds to human GITR comprises a VL comprising an amino acid sequence at least 75%, 80%, 85%, 90%, 95%, or 99% identical to an amino acid sequence selected from the group consisting of SEQ ID NOs: 19, 21, and 23. In one aspect, the antigen-binding domain that specifically binds to human GITR comprises a VL comprising an amino acid sequence selected from the group consisting of SEQ ID NOs: 19, 21, and 23. In one aspect, the antigen-binding domain that specifically binds to human GITR comprises a VL comprising the amino acid sequence of SEQ ID NO:19.

In one aspect, the antigen-binding domain that specifically binds to human GITR comprises a light chain comprising the amino acid sequence of SEQ ID NO: 37. In one aspect, the antigen-binding domain that specifically binds to human GITR comprises a light chain comprising the amino acid sequence of SEQ ID NO: 38.

In one aspect, the antigen-binding domain that specifically binds to human GITR comprises a VL comprising an amino acid sequence derived from a human IGKV4-1 germline sequence.

In one aspect, the antigen-binding domain that specifically binds to human GITR comprises VH and VL sequences set forth in SEQ ID NOs: 18 and 19, SEQ ID NOs: 20 and 21, SEQ ID NOs: 22 and 23, or SEQ ID NOs: 24 and 23, respectively. In one aspect, the antigen-binding domain that specifically binds to human GITR comprises a VH comprising the sequence set forth in SEQ ID NO:18 and a VL comprising the sequence set forth in SEQ ID NO:19.

In one aspect, the antigen-binding domain that specifically binds to human GITR comprises one heavy chain and one light chain.

In one aspect, an isolated antibody that specifically binds to human GITR comprises an antigen-binding domain provided herein that specifically binds to human GITR and is selected from the group consisting of a Fab, Fab′, F(ab′)₂, and say fragment.

In one aspect, the first antigen-binding domain comprises a first human IgG₁ heavy chain and the second antigen-binding domain comprises a second human IgG₁ heavy chain, wherein the first and second heavy chains comprise an identical mutation selected from the group consisting of N297A, N297Q, D265A, and a combination thereof, numbered according to the EU numbering system. In one aspect, the first antigen-binding domain comprises a first human IgG₁ heavy chain and the second antigen-binding domain comprises a second human IgG₁ heavy chain, wherein the first and second heavy chains comprise an identical mutation selected from the group consisting of D265A, P329A, and a combination thereof, numbered according to the EU numbering system.

In one aspect, the first antigen-binding domain comprises a first human IgG₂ heavy chain and the second antigen-binding domain comprises a second human IgG₂ heavy chain, wherein the first and second heavy chains comprise a C127S mutation, numbered according to Kabat. In one aspect, the first antigen-binding domain comprises a first human IgG₄ heavy chain and the second antigen-binding domain comprises a second human IgG₄ heavy chain, wherein the first and second heavy chains comprise a S228P mutation, numbered according to the EU numbering system. In one aspect, the first and second heavy chains are human IgG₁ heavy chains, wherein the first and second heavy chains comprise an identical mutation selected from the group consisting of N297A, N297Q, D265A, and a combination thereof, numbered according to the EU numbering system. In one aspect, the first and second heavy chains are human IgG₁ heavy chains, wherein the first and second heavy chains comprise an identical mutation selected from the group consisting of D265A, P329A, and a combination thereof, numbered according to the EU numbering system. In one aspect, the first and second heavy chains are human IgG₂ heavy chains, wherein the first and second heavy chains comprise a C1275 mutation, numbered according to Kabat. In one aspect, the first and second heavy chains are human IgG₄ heavy chains, wherein the first and second heavy chains comprise a S228P mutation, numbered according to the EU numbering system.

In one aspect, the antibody is antagonistic to human GITR. In one aspect, the antibody deactivates, reduces, or inhibits an activity of human GITR. In one aspect, the antibody inhibits or reduces binding of human GITR to human GITR ligand. In one aspect, the antibody inhibits or reduces human GITR signaling. In one aspect, the antibody inhibits or reduces human GITR signaling induced by human GITR ligand.

In one aspect, the antibody decreases CD4+ T cell proliferation induced by synovial fluid from rheumatoid arthritis patients. In one aspect, the antibody increases survival of NOG mice transplanted with human PBMCs. In one aspect, the antibody increases proliferation of regulatory T cells in a GVHD model.

In one aspect, the antibody further comprises a detectable label.

In one aspect, provided herein is a pharmaceutical composition comprising an antibody that specifically binds to GITR (e.g., human GITR) provided herein and a pharmaceutically acceptable excipient.

In one aspect, provided herein is a method of modulating an immune response in a subject comprising administering to the subject an effective amount of an antibody that specifically binds to GITR (e.g., human GITR) provided herein or a pharmaceutical composition provided herein. In one aspect, modulating an immune response comprises reducing or inhibiting the immune response in the subject.

In one aspect, provided herein is a method of treating an autoimmune or inflammatory disease or disorder in a subject comprising administering to the subject an effective amount of an antibody that specifically binds to GITR (e.g., human GITR) provided herein or a pharmaceutical composition provided herein. In one aspect, the disease or disorder is selected from the group consisting of transplant rejection, graft-versus-host disease, vasculitis, asthma, rheumatoid arthritis, dermatitis, inflammatory bowel disease, uveitis, lupus, colitis, diabetes, multiple sclerosis, and airway inflammation.

In one aspect, provided herein is a method of treating an infectious disease in a subject comprising administering an effective amount of an antibody that specifically binds to GITR (e.g., human GITR) provided herein or a pharmaceutical composition provided herein.

In one aspect, the subject is human.

In one aspect, provided herein is a method for detecting GITR in a sample comprising contacting the sample with an antibody that specifically binds to GITR (e.g., human GITR) provided herein.

In one aspect, provided herein is a kit comprising an antibody that specifically binds to GITR (e.g., human GITR) provided herein or a pharmaceutical composition provided herein and a) a detection reagent, b) a GITR antigen, c) a notice that reflects approval for use or sale for human administration, or d) a combination thereof.

In one aspect, provided herein is a method of reducing or inhibiting an immune response in a subject, wherein the method comprises administering to the subject an effective amount of an isolated antibody that specifically binds to human GITR, wherein the antibody comprises: (i) an antigen-binding domain that specifically binds to human GITR, comprising: (a) a first heavy chain comprising a first heavy chain variable domain (VH) comprising a VH complementarity determining region (CDR) 1 comprising the amino acid sequence of X₁YX₂1MX₃(SEQ ID NO:87), wherein X₁ is D, E or G; X₂ is A or V, and X₃ is Y or H; a VH-CDR2 comprising the amino acid sequence of X₁IX₂T X₃SGX₄X₅X₆YNQKFX₇X₈ (SEQ ID NO:88), wherein X₁ is V or L, X₂ is R, K or Q, X₃ is Y or F, X₄ is D, E or G, X₅ is V or L, X₆ is T or S, X₇ is K, R or Q, and X₈ is D, E or G; and a VH-CDR3 comprising the amino acid sequence of SGTVRGFAY (SEQ ID NO:3); and (b) a first light chain comprising a first light chain variable domain (VL) comprising a VL-CDR1 comprising the amino acid sequence of KSSQSLLNSX₁NQKNYLX₂(SEQ ID NO:90), wherein X₁ is G or S, and X₂ is I or S; a VL-CDR2 comprising the amino acid sequence of WASTRES (SEQ ID NO:5); and a VL-CDR3 comprising the amino acid sequence of QNX₁YSX₂PYT (SEQ ID NO:92), wherein X₁ is D or E; and X₂ is Y, F or S; and (ii) a second heavy chain or a fragment thereof; and wherein the antibody is antagonistic to human GITR.

In one aspect, provided herein is a method of treating an autoimmune or inflammatory disease or disorder in a subject, wherein the method comprises administering to the subject an effective amount of an isolated antibody that specifically binds to human GITR, wherein the antibody comprises: (i) an antigen-binding domain that specifically binds to human GITR, comprising: (a) a first heavy chain comprising a first heavy chain variable domain (VH) comprising a VH complementarity determining region (CDR) 1 comprising the amino acid sequence of X₁YX₂MX₃(SEQ NO:87), wherein X₁ is D, E or G; X₂ is A or V, and X₃ is Y or H; a VH-CDR2 comprising the amino acid sequence of X₁IX₂TX₃SGX₄X₅X₆YNQKFX₇X₈(SEQ ID NO:88), wherein X₁ is V or L, X₂ is R, K or Q, X₃ is Y or F, X₄ is D, E or G, X₅ is V or L, X₆ is T or S, X-₇ is K, R or Q, and X₈ is D, E or G; and a VH-CDR3 comprising the amino acid sequence of SGTVRGFAY (SEQ ID NO:3); and (b) a first light chain comprising a first light chain variable domain (VL) comprising a VL-CDR1 comprising the amino acid sequence of KSSQSLLNSX₁NQKNYLX₂(SEQ ID NO:90), wherein X₁ is G or S, and X₂ is T or S; a VL-CDR2 comprising the amino acid sequence of WASTRES (SEQ ID NO:5); and a VL-CDR3 comprising the amino acid sequence of QNX₁YSX₂PYT (SEQ ID NO:92), wherein X₁ is D or E; and X₂ is Y, F or S; and (ii) a second heavy chain or a fragment thereof; wherein the antibody is antagonistic to human GITR.

In one aspect, the second heavy chain or a fragment thereof comprises a second heavy chain variable domain and a second heavy chain constant domain.

In one aspect, the antibody further comprises a second light chain comprising a second light chain variable domain and a second light chain constant domain.

In one aspect, the antigen-binding domain that specifically binds to human GITR comprises VH-CDR1, VH-CDR2, VH-CDR3, VL-CDR1, VL-CDR2, and VL-CDR3 sequences comprising the amino acid sequences of SEQ ID NOs: 1-6, respectively.

In one aspect, the antigen-binding domain that specifically binds to human GITR comprises VH-CDR1, VH-CDR2, and VH-CDR3 sequences set forth in SEQ ID NOs: 7, 10, and 3; SEQ ID NOs: 8, 11, and 3; SEQ ID NOs: 9, and 3; or SEQ ID NOs: 9, 13, and 3, respectively; and/or VL-CDR1, VL-CDR2, and VL-CDR3 sequences set forth in SEQ ID NOs: 14, 5, and 16; or SEQ ID NOs: 15, 5, and 17, respectively.

In one aspect, the antigen-binding domain that specifically binds to human GITR comprises a VH and a VL, wherein the VH comprises an amino acid sequence selected from the group consisting of SEQ ID NOs: 18, 20, 22, 24, and 25.

In one aspect, the antigen-binding domain that specifically binds to human GITR comprises a VH and a VL, wherein the VL comprises an amino acid sequence selected from the group consisting of SEQ ID NOs: 19, 21, 23, and 26,

In one aspect, the antigen-binding domain that specifically binds to human GITR comprises VH and VL sequences set forth in SEQ ID NOs: 18 and 19, SEQ ID NOs: 20 and 21, SEQ ID NOs: 22 and 23, or SEQ ID NOs: 24 and 23, respectively.

In one aspect, the first and second heavy chains comprise an identical mutation selected from the group consisting of N297A, N297Q, D265A, and a combination thereof, numbered according to the EU numbering system. In one aspect, the first and second heavy chains comprise the identical mutation of N297A, numbered according to the EU numbering system.

In one aspect, the antigen-binding domain that specifically binds to human GITR binds to the same epitope of human GITR as an antibody comprising a VH comprising the amino acid sequence of SEQ ID NO:18 and a VL comprising the amino acid sequence of SEQ ID NO:19.

In one aspect, the antigen-binding domain that specifically binds to human GITR exhibits, as compared to binding to a human GITR sequence of residues 26 to 241 of SEQ ID NO:41, reduced or absent binding to a protein identical to residues 26 to 241 of SEQ ID NO:41 except for the presence of a D60A or G63A amino acid substitution, numbered according to SEQ ID NO:41.

In one aspect, the antigen-binding domain that specifically binds to human GITR specifically binds to an epitope of GITR comprising at least one amino acid in residues 60-63 of SEQ ID NO:41.

In one aspect, the antigen-binding domain that specifically binds to human GITR specifically binds to each of i) human GITR, comprising amino acid residues 26 to 241 of SEQ ID NO:41; and ii) a variant of cynomolgus GITR, said variant comprising amino acid residues 26-234 of SEQ ID NO:46, wherein the antigen-binding domain that specifically binds to human GITR does not specifically bind to cynomolgus GITR comprising amino acid residues 26-234 of SEQ ID NO:44.

### 6. BRIEF DESCRIPTION OF THE FIGURES

FIG. 1A and 1B: FIG. 1A depicts NF-κB-luciferase signal from Jurkat-huGITR-NF-κB-luciferase reporter cells triggered by trimeric GITRL. FIG. 1B is a graph showing the luciferase signal induced by pab1876w or an isotype control antibody. Relative light units (RLU) are plotted against a dose titration of GITRL or antibody concentrations.

FIGS. 2A and 2B are results of a reporter assay where Jurkat-huGITR-NF-κB-luciferase reporter cells were incubated with GITR ligand (GITRL)-expressing cells and soluble pab1876w or an isotype control antibody. FIG. 2A is a graph showing % GITRL activity (GITRL-induced activation normalized as a percent of maximal stimulation) plotted against a range of antibody concentrations. FIG. 2B is a bar graph showing % GITRL activity at 5 μg/ml antibody concentration for the indicated treatment groups. FIG. 2C is a graph showing % GITRL activity over a range of antibody concentrations from a study in which Jurkat-huGITR-NF-κB-luciferase reporter cells were incubated with cross-linked recombinant GITRL and soluble pab1876w or an isotype control antibody.

FIG. 3 is a histogram showing the loss of binding of 1624-5 pre-B cells expressing the chimeric parental 231-32-15 antibody to biotinylated GITR (GITR-bio) when GITR-bio was pre-incubated with chimeric parental 231-32-15, pab1875 or pab1876 antibodies. The FIG. 3 right-hand profile depicts the binding of 1624-5 pre-B cells expressing the chimeric parental 231-32-15 antibody to GITR-bio. In the left-hand profile, however, there is loss of binding of 1624-5 cells expressing the chimeric parental 231-32-15 antibody to GITR-bio following pre-incubation of GITR-bio with either the chimeric parental 231-32-15, pab1875 or pab1876 antibodies.

FIG. 4 shows the results of an epitope competition assay measured by surface plasmon resonance (BIAcore® T100/200). GITR antigen was immobilized on a CM5 sensor chip and the anti-GITR antibodies applied at a concentration of 300 nM. Chimeric parental 231-32-15 antibody was applied first followed by the application of the murine antibody 6C8.

FIGS. 5A and 5B are the results of an epitope mapping experiment using a cellular library expressing GITR variants generated by error prone PCR. Shown in FIGS. 5A and 5B is an alignment of sequences from the GITR variants that bind to a polyclonal anti-GITR antibody but do not bind to the anti-GITR chimeric parental 231-32-15 antibody.

FIGS. 6A and 6B are the result of an epitope mapping experiment using alanine scanning. The following positions in human GITR (numbered according to SEQ ID NO: 41) were separately mutated to an Alanine: P28A, T29A, G30A, G31 A, P32A, T54A, T55A, R56A, C57A, C58A, R59A, D60A, Y61A, P62A, G63A, E64A, E65A, C66A, C67A, S68A, E69A, W70A, D71A, C72A, M73A, C74A, V75A and Q76A. The antibodies tested in the experiment shown in FIG. 6A included: the monoclonal anti-GITR antibodies pab1876, pab1967, pab1975, pab1979 and m6C8; and a polyclonal anti-GITR antibody (AF689, R&D systems), FIG. 6A is a table summarizing the binding of pab1876, pab1967, pab1975, pab1979 and the reference antibody m6C8 to1.624-5 cells expressing human GITR alanine mutants. FIG. 6B is a set of flow cytometry plots showing the staining of 1624-5 cells expressing wild type human GITR, D60A mutant, or G63A mutant using the monoclonal antibody 231-32-15, pab1876, or m6C8, or a polyclonal antibody. The percentage of GITR positive cells is indicated in each plot.

FIG. 7A is a sequence alignment of human GITR, V1M cynomolgus GITR, and V1M/Q62P/S63G cynomolgus GITR, highlighting the positions 62 and 63 where two amino acids from cynomolgus GITR (GlnSer) were replaced by corresponding residues in human GITR (ProGly). FIG. 7B is a set of flow cytometry plots showing the staining of 1624-5 cells expressing human GITR, V1M cynomolgus GITR, or V1M/Q62P/S63G cynomolgus GITR using the monoclonal antibody 231-32-15, pab1876, or m6C8, or a polyclonal anti-GITR antibody.

### 7. DETAILED DESCRIPTION

Provided herein are antibodies that specifically bind to GITR (e.g., human GITR). For example, in one aspect, provided herein are antibodies that specifically bind to GITR (e.g., human GITR) and deactivate, reduce, or inhibit one or more GITR activities. In a specific embodiment, the antibodies are isolated.

Also provided are isolated nucleic acids (polynucleotides), such as complementary DNA (cDNA), encoding such antibodies. Further provided are vectors (e.g., expression vectors) and cells (e.g., host cells) comprising nucleic acids (polynucleotides) encoding such antibodies. Also provided are methods of making such antibodies. In other aspects, provided herein are methods and uses for deactivating, reducing, or inhibiting GITR (e.g., human GITR) activity, and treating certain conditions, such as inflammatory or autoimmune diseases and disorders. Related compositions (e.g., pharmaceutical compositions), kits, and detection methods are also provided.

### 7.1 Terminology

As used herein, the terms “about” and “approximately,” when used to modify a numeric value or numeric range, indicate that deviations of 5% to 10% above and 5% to 10% below the value or range remain within the intended meaning of the recited value or range.

As used herein, the terms “antibody” and “antibodies” are terms of art and can be used interchangeably herein and refer to a molecule with an antigen-binding site that specifically binds an antigen.

Antibodies can include, for example, monoclonal antibodies, recombinantly produced antibodies, human antibodies, humanized antibodies, resurfaced antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), camelized antibodies, affybodies, Fab fragments, F(ab′)₂ fragments, disulfide-linked. Fvs (sdFv), anti-idiotypic (anti-Id) antibodies (including, e.g., anti-anti-Id antibodies), bispecific antibodies, and multi-specific antibodies. In certain embodiments, antibodies described herein refer to polyclonal antibody populations. Antibodies can be of any type (e.g., IgG, IgE, IgM, IgD, IgA, or IgY), any class (e.g., IgG₁, IgG₂, IgG₃, IgG₄, IgA₁, or IgA₂), or any subclass (e.g., IgG₂ₐ or IgG_{2b}) of immunoglobulin molecule. In certain embodiments, antibodies described herein are IgG antibodies, or a class (e.g., human IgG₁, IgG₂, or IgG₄) or subclass thereof. In a specific embodiment, the antibody is a humanized monoclonal antibody. In another specific embodiment, the antibody is a human monoclonal antibody, e.g., that is an immunoglobulin. In certain embodiments, an antibody described herein is an IgG₁, IgG₂, or IgG₄ antibody.

As used herein, the terms “antigen-binding domain,” “antigen-binding region,” “antigen-binding site,” and similar terms refer to the portion of antibody molecules which comprises the amino acid residues that confer on the antibody molecule its specificity for the antigen (e.g., the complementarity determining regions (“CDR”)). The antigen-binding region can be derived from any animal species, such as rodents (e.g., mouse, rat, or hamster) and humans.

As used herein, the term “antigen-binding domain that does not specifically bind to an antigen expressed by a human immune cell” means that the antigen-binding domain does not bind to an antigen expressed by any cell of hematopoietic origin that plays a role in the human immune response. Human immune cells include lymphocytes, such as B cells and T cells; natural killer cells; and myeloid cells, such as monocytes, macrophages, eosinophils, mast cells, basophils, and granulocytes. For example, such a binding domain would not bind to GITR or any other members of the TNF receptor superfamily that are expressed by a human immune cell. However, the antigen-binding domain can bind to an antigen such as, but not limited to, an antigen expressed in other organisms and not humans(i.e., a non-human antigen); an antigen that is not expressed by wild-type human cells; or a viral antigen, including, but not limited to, an antigen from a virus that does not infect human cells, or a viral antigen that is absent in an uninfected human immune cell.

As used herein, the terms “variable region” or “variable domain” are used interchangeably and are common in the art. The variable region typically refers to a portion of an antibody, generally, a portion of a light or heavy chain, typically about the amino-terminal 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

The terms “VL” and “VL domain” are used interchangeably to refer to the light chain variable region of an antibody.

The terms “VH” and “VH domain” are used interchangeably to refer to the heavy chain variable region of an antibody.

The term “Kabat numbering” and like terms are recognized in the art and refer to a system of numbering amino acid residues in the heavy and light chain variable regions of an antibody, or an antigen-binding portion thereof. In certain aspects, the CDRs of an antibody can be determined according to the Kabat numbering system (see, e.g., Kabat, E A & Wu, T T (1971) Ann NY Acad Sci 190: 382-391 and Kabat, E A et al., (1991) Sequences of Proteins of immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). In a specific embodiment, the CDRs of the antibodies described herein have been determined according to the Kabat numbering scheme.

As used herein, the term “constant region” or “constant domain” are interchangeable and have its meaning common in the art. The constant region is an antibody portion, e.g., a carboxyl terminal portion of a light and/or heavy chain which is not directly involved in binding of an antibody to antigen but which can exhibit various effector functions, such as interaction with the Fe receptor. The constant region of an immunoglobulin molecule generally has a more conserved amino acid sequence relative to an immunoglobulin variable domain.

As used herein, the term “heavy chain” when used in reference to an antibody can refer to any distinct type, e.g., alpha (α), delta (δ), epsilon (ε), gamma (γ), and mu (μ), based on the amino acid sequence of the constant domain, which give rise to IgA, IgD, IgE, IgG, and IgM classes of antibodies, respectively, including subclasses of IgG, e.g., IgG₁, IgG₂, IgG₃, and IgG₄.

As used herein, the term “light chain” when used in reference to an antibody can refer to any distinct type, e.g., kappa (κ) or lambda (λ) based on the amino acid sequence of the constant domains. Light chain amino acid sequences are well known in the art. In specific embodiments, the light chain is a human light chain.

As used herein, the term “EU numbering system” refers to the EU numbering convention for the constant regions of an antibody, as described in Edelman, G. M. et al., Proc. Natl. Acad. USA, 63, 78-85 (1969) and Kabat et al, Sequences of Proteins of Immunological Interest, U.S. Dept. Health and Human Services, 5th edition, 1991, each of which is herein incorporated by reference in its entirety.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ, whereas K_{A} is calculated from the quotient of kₒₙ/k_{off}. kₒₙ refers to the association rate constant of, e.g., an antibody to an antigen, and k_{off} refers to the dissociation of, e.g., an antibody to an antigen. The kₒₙ and k_{off} can be determined by techniques known to one of ordinary skill in the art, such as BIAcore® or KinExA.

As used herein, a “conservative amino acid substitution” is one in which the amino acid residue is replaced with an amino acid residue having a similar side chain. Families of amino acid residues having side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In certain embodiments, one or more amino acid residues within a CDR(s) or within a framework region(s) of an antibody can be replaced with an amino acid residue with a similar side chain.

As used herein, an “epitope” is a term in the art and refers to a localized region of an antigen to which an antibody can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In certain embodiments, the epitope to which an antibody binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giegé, R et at., Acta Crystallogr D Biol Crystallogr 50(Pt 4): 339-350 (1994); McPherson, A, Eur J Biochem 189: 1-23 (1990); Chayen, N E, Structure 5: 1269-1274 (1997); McPherson, A, J Bio Chem 251: 6300-6303 (1976)). Antibody:antigen crystals can be studied using well known X-ray diffraction techniques and can be refined using computer software such as X-PLOR (Yale University, 1992, distributed by Molecular Simulations, Inc.; see, e.g., Meth Enzymol (1985) volumes 114 & 115, eds. Wyckoff, H W et al.,; U.S. 2004/0014194), and BUSTER (Bricogne, G, Acta Crystallogr D Biol Crystallogr 49(Pt 1): 37-60 (1993); Bricogne, G, Meth Enzymol 276A:361-423 (1997), ed Carter, C W; Roversi, P et al., Acta Crystallogr D Biol Crysollogr 56(Pt 10):1316-1323 (2000)). Mutagenesis mapping studies can be accomplished using any method known to one of skill in the art. See, e.g., Champe, M et al., J Biol Chem 270: 1388-1394 (1995) and Cunningham, B C & Wells, J A Science 244: 1081-1085 (1989) for a description of mutagenesis techniques, including alanine scanning mutagenesis techniques. In a specific embodiment, the epitope of an antibody is determined using alanine scanning mutagenesis studies.

As used herein, the terms “immunospecifically binds,” “immunospecifically recognizes,” “specifically binds,” and “specifically recognizes” are analogous terms in the context of antibodies and refer to molecules that bind to an antigen (e.g., epitope or immune complex) as such binding is understood by one skilled in the art. For example, a molecule that specifically binds to an antigen can bind to other peptides or polypeptides, generally with lower affinity as determined by, e.g., immunoassays, BIAcore®, KinExA. 3000 instrument (Sapidyne Instruments, Boise, Id.), or other assays known in the art. In a specific embodiment, molecules that immunospecifically bind to an antigen bind to the antigen with a K_{A} that is at least 2 logs, 2,5 logs, 3 logs, 4 logs or greater than the K_{A} when the molecules bind non-specifically to another antigen. In the context of antibodies with an anti-GITR antigen-binding domain and a second antigen-binding domain (e.g., a second antigen-binding domain that does not specifically bind to an antigen expressed by a human immune cell), the terms “immunospecifically binds,” “immunospecifically recognizes,” “specifically binds,” and “specifically recognizes” refer to antibodies that have distinct specificities for more than one antigen (i.e., GITR and the antigen associated with the second antigen-binding domain).

In another specific embodiment, antigen-binding domains that immunospecifically bind to an antigen do not cross react with other proteins under similar binding conditions. In another specific embodiment, antigen-binding domains that immunospecifically bind to GITR antigen do not cross react with other non-GITR proteins. In a specific embodiment, provided herein is an antibody containing an antigen-binding domain that binds to GITR with higher affinity than to another unrelated antigen. In certain embodiments, provided herein is an antibody containing an antigen-binding domain that binds to GITR (e.g., human GITR) with a 20%, 25%, 30%, 35%, 40%, 45%, 50%, 55%, 60%, 65%, 70%, 75%, 80%, 85%, 90%, 95% or higher affinity than to another, unrelated antigen as measured by, e.g., a radioimmunoassay, surface plasmon resonance, or kinetic exclusion assay. In a specific embodiment, the extent of binding of an anti-GITR antigen-binding domain described herein to an unrelated, non-GITR protein is less than 10%, 15%, or 20% of the binding of the antigen-finding domain to GITR protein as measured by, e.g., a radioimmunoassay.

In a specific embodiment, provided herein is an antibody containing an antigen-binding domain that binds to human GITR with higher affinity than to another species of GITR. In certain embodiments, provided herein is an antibody containing an antigen-binding domain that binds to human GITR with a 5%, 10%, 15%, 20%, 25%, 35%, 40%, 45%, 50%, 55%, 60%, 65%, 70% or higher affinity than to another species of GITR as measured by, e.g., a radioimmunoassay, surface plasmon resonance, or kinetic exclusion assay. In a specific embodiment, an antibody described herein, which binds to human GITR will bind to another species of GITR with less than 10%, 15%, or 20% of the binding of the antibody to the human GITR protein as measured by, e.g., a radioimmunoassay, surface plasmon resonance, or kinetic exclusion assay.

As used herein, the terms “glucocorticoid-induced INF receptor,” “glucocorticoid-induced TNF receptor-related protein,” “glucocorticoid-induced TNF receptor family-related protein,” or “GITR” or “GITR. polypeptide” refer to GITR including, but not limited to, native GITR, an isoform of GITR, or an interspecies GITR homolog of GITR. GITR is also known as activation-inducible TNFR family receptor (AITR), GITR-D, CD357, and tumor necrosis factor receptor superfamily member 18 (TNFRSF18). GenBank™ accession numbers BC152381 and BC152386 provide human GITR nucleic acid sequences. Swiss-Prot accession number Q9Y5U5-1 (INR18_HUMAN; SEQ ID NO:41) and GenBank™ accession number NP_004186 provide exemplary human GITR amino acid sequences for isoform 1. This amino acid sequence is 241 amino acids in length with the first 25 amino acid residues encoding the signal sequence. Isoform 1 is a type I membrane protein. An exemplary mature amino acid sequence of human GITR is provided as SEQ ID NO:40. In contrast, isoform 2 is a secreted form of human GITR and is approximately 255 amino acids in length. Swiss-Prot accession number Q9Y5U5-2 and. GenBank™ accession number NP_683699 provide exemplary human GITR amino acid sequences for isoform 2. Isoform 3 of human GITR is approximately 234 amino acids in length. Swiss-Prot accession number Q9Y5U5-3 and GenBankTm accession number NP_683700 (isoform 3 precursor) provide exemplary human GITR amino acid sequences for isoform 3. In a specific embodiment, the GITR, is human GITR. In another specific embodiment, the GITR is human GITR isoform 1 (SEQ NO:41). In certain embodiments, the GITR is human isoform 2 (SEQ ID NO:42) or human GITR isoform 3 (SEQ ID NO:43). Human GITR is designated GeneID: 8784 by Entrez Gene. SEQ ID NO:44 provides the cynomolgus GITR amino acid sequence, and amino acids 26-234 of SEQ ID NO:44 represent the mature form of cynomolgus GITR. As used herein, the term “human GITR” refers to GITR comprising the polypeptide sequence of SEQ ID NO:40.

As used herein, the terms “GITR ligand” and “GITRL” refer to glucocorticoid-induced. TNFR-related protein ligand. GITRL is otherwise known as activation-induced TNF-related ligand (AITRL) and tumor necrosis factor ligand superfamily member 18 (TNISF18). GenBank™ accession number AF125303 provides an exemplary human GITRL nucleic acid sequence. GenBank™ accession number NP_005083 and Swiss-Prot accession number Q9UNG2 provide exemplary human GITRL amino acid sequences.

As used herein, the term “host cell” can be any type of cell, e.g., a primary cell, a cell in culture, or a cell from a cell line. In specific embodiments, the term “host cell” refers to a cell transfected with a nucleic acid molecule and the progeny or potential progeny of such a cell. Progeny of such a cell are not necessarily identical to the parent cell transfected with the nucleic acid molecule, e.g., due to mutations or environmental influences that may occur in succeeding generations or integration of the nucleic acid molecule into the host cell genome.

As used herein, the term “effective amount” in the context of the administration of a therapy to a subject refers to the amount of a therapy that achieves a desired prophylactic or therapeutic effect. Examples of effective amounts are provided in Section 7.5, infra.

As used herein, the terms “subject” and “patient” are used interchangeably. The subject can be an animal. In some embodiments, the subject is a mammal such as a non-primate (e.g., cow, pig, horse, cat, dog, rat, etc.) or a primate (e.g., monkey or human) most preferably a human. In some embodiments, the subject is a cynomolgus monkey. In certain embodiments, such terms refer to a non-human animal (e.g., a non-human animal such as a pig, horse, cow, cat, or dog). In some embodiments, such terms refer to a pet or farm animal. In specific embodiments, such terms refer to a human.

As used herein, the binding between a test antibody and a first antigen is “substantially weakened” relative to the binding between the test antibody and a second antigen if the binding between the test antibody and the first antigen is reduced by at least 30%, 40%, 50%, 60%, 70%, or 80% relative to the binding between the test antibody and the second antigen, e.g., in a given experiment, or using mean values from multiple experiments, as assessed by, e.g., an assay comprising the following steps: (a) expressing on the surface of cells (e.g., 1624-5 cells) the first antigen or the second antigen; (b) staining the cells expressing the first antigen or the second antigen using, e.g., 2 μg/ml of the test antibody or a polyclonal antibody in a flow cytometry analysis and recording mean fluorescence intensity (MFI) values, e.g., as the mean from more than one measurement, wherein the polyclonal antibody recognizes both the first antigen and the second antigen; (c) dividing the MFI value of the test antibody for the cells expressing the second antigen by the MFI value of the polyclonal antibody for the cells expressing the second antigen (MFI ratio₂); (d) dividing the MFI value of the test antibody for the cells expressing the first antigen by the MFI value of the polyclonal antibody for the cells expressing the first antigen (MFI ratio₁); and (e) determining the percentage of reduction in binding by calculating 100%*(1-(MFI ratio₁/MFI ratio₂)).

The determination of “percent identity” between two sequences (e.g., amino acid sequences or nucleic acid sequences) can also be accomplished using a mathematical algorithm. A specific, non-limiting example of a mathematical algorithm utilized for the comparison of two sequences is the algorithm of Karlin, S & Altschul, S F, PNAS 87: 2264-2268 (1990), modified as in Karlin S & Altschul S F PNAS 90: 5873-5877 (1993). Such an algorithm is incorporated into the NBLAST and XBLAST programs of Altschul, S F et at., J Mol Biol 215: 403 (1990). BLAST nucleotide searches can be performed with the NBLAST nucleotide program parameters set, e.g., for score=100, wordlength=12 to obtain nucleotide sequences homologous to a nucleic acid molecules described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul, S F et al., Nuc Acids Res 25: 3389 3402 (1997). Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology information (NCBI) on the worldwide web, ncbi.nlm.nih.gov). Another specific, non-limiting example of a mathematical algorithm utilized for the comparison of sequences is the algorithm of Myers and Miller, CABIOS 4:11 17 (1988). Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

### 7.2 Antibodies

The activation of GITR signaling depends on receptor clustering to form higher order receptor complexes that efficiently recruit apical adapter proteins to drive intracellular signal transduction. Without being bound by theory, an anti-GITR agonist antibody may mediate receptor clustering through bivalent antibody arms (i.e., two antibody arms that each bind GITR antigen) and/or through Fc-Fc receptor (FcR) co-engagement on accessory myeloid or lymphoid cells. Consequently, one approach for developing an anti-GITR antagonist antibody is to select an antibody that competes with GITR ligand (GITRL) for binding to GITR, diminish or eliminate the binding of the Fc region of an antibody to Fc receptors, and/or adopt a monovalent antibody format. The monovalent antibody format can include antibodies that are structurally monovalent, such as, but not limited to, anti-GITR antibodies comprising only one antigen-binding domain (e.g., only one Fab arm), or antibodies comprising only one antigen-binding domain that binds to GITR (e.g., human GITR) that is paired with a heavy chain or that is paired with a fragment of a heavy chain (e.g., an Fc fragment). The monovalent antibody format can also include antibodies that are functionally monovalent, for example, antibodies comprising only one antigen-binding domain that binds to GITR (e.g., human GITR) that is paired with a second antigen-binding domain that does not bind to an antigen expressed by a human immune cell (i.e., the antibody comprises two antigen-binding domains, but only one antigen-binding domain binds to GITR).

In a specific aspect, provided herein are antagonist antibodies, which immunospecifically bind to GITR (e.g., human GITR).

### 7.2.1 Antigen-Binding Domains that Bind to GITR

In certain embodiments, an antigen-binding domain provided herein that specifically binds to GITR contains a combination of heavy chain CDRs and light chain CDRs as shown in Tables 1 and 2, respectively.

TABLE 1
Heavy chain CDR sequences of exemplary anti-GITR antibodies*

| Antibody | HCDR1 (SEQ ID NO:) | HCDR2 (SEQ ID NO:) | HCDR3 (SEQ ID NO:) |
|---|---|---|---|
| pab1876w | DYAMY (7) | VITRYSGDVTYNQKFKD (10) | SGTVRGFAY (3) |
| pab1967w | GYAMH (8) | LIRTYSGGVSYNQKFRE (11) | SGTVRGFAY (3) |
| pab1975w | EYAMH (9) | LIRTYSGGVSYNQKFQG (12) | SGTVRGFAY (3) |
| pab1979w | EYAMH (9) | VIRTYSGGVSYNQKFQE (13) | SGTVRGFAY (3) |

*The VH CDRs in Table 1 are determined according to Kabat.

TABLE 2
Light chain CDR sequences of exemplary anti-GITR antibodies*

| Antibody | LCDR1 (SEQ ID NO:) | LCDR2 (SEQ ID NO:) | LCDR3 (SEQ ID NO:) |
|---|---|---|---|
| pab1876w | KSSQSLLNSGNQKNYLT (14) | WASTRES (5) | QNDYSYPYT (16) |
| pab1967w | KSSQSLLNSSNQKNYLT (15) | WASTRES (5) | QNEYSFPYT (17) |
| pab1975w | KSSQSLLNSGNQKNYLT (14) | WASTRES (5) | QNDYSYPYT (16) |
| pab1979w | KSSQSLLNSGNQKNYLT (14) | WASTRES (5) | QNDYSYPYT (16) |

*The VL CDRs in Table 2 are determined according to Kabat.

In certain embodiments, an antigen-binding domain provided herein that specifically binds to GITR contains a combination of VI-I and VL sequences, as shown in Table 3.

TABLE 3
VH and VL sequences of exemplary anti-GITR antibodies

| Antibody | VH (SEQ ID NO:) | VL (SEQ ID NO:) |
|---|---|---|
| pab1876w | 18 | 19 |
| pab1967w | 20 | 21 |
| pab1975w | 22 | 23 |
| pab1979w | 24 | 23 |

In a particular embodiment, an antigen-binding domain described herein, which specifically binds to GITR (e.g., human Gym), comprises a light chain variable region (VL) comprising:
(a) a VL-CDR1 comprising the amino acid sequence of KSSQSLLNSX₁NQKNYLX₂ (SEQ ID NO: 90), wherein X₁ is G or S; and X₂ is T or S;
(b) a VL-CDR2 comprising the amino acid sequence of WASTRES (SEC) ID NO: 5); and
(c) a VL-CDR3 comprising the amino acid sequence of QNX₁YSX₂PYT (SEQ ID NO: 92), wherein X₁ is D or E; and X₂ is Y, F or S, as shown in Table 4.

In another embodiment, a GITR antigen-binding domain described herein, which specifically binds to GITR (e.g., human GITR), comprises a comprising a heavy chain variable region (VH) comprising:
(a) a VH-CDR1 comprising the amino acid sequence of X₁YX₂MX₃ (SEQ ID NO: 87), wherein X₁ is D, E or G; X₂ is A or V; and X₃ is Y or H;
(b) a VH-CDR2 comprising the amino acid sequence of X₁IX₂TX₃SGX₄X₅X₆YNQKFX₇X₈ (SEQ ID NO: 88), wherein X₁ is V or L; X₂ is R, K or Q; X₃ is Y or F; X₄ is D, E or G; X₅ is or L; X₆ is T or S; X-₇ is K, R or Q; and X₈ is D, E or G;
(c) a VH-CDR3 comprising the amino acid sequence of SGTVRGFAY (SEQ ID NO: 3), as shown in Table 5.

In another particular embodiment, an antigen-binding domain described herein, which specifically binds to GITR (e.g., human GITR), comprises a light chain variable region (VL) comprising:
(a) a VL-CDR1 comprising the amino acid sequence of KSSQSLLNSX₁NQKNYLX₂ (SEQ ID NO: 4), wherein X₁ is G or S;
(b) a VL-CDR2 comprising the amino acid sequence of WASTRES (SEQ ID NO: 5); and
(c) a VL-CDR3 comprising the amino acid sequence of QNX₁YSX₂PYT (SEQ ID NO: 6), wherein X₁ is D or E; and X₂ is Y or F, as shown in Table 4.

In another embodiment, a GITR antigen-binding domain described herein, which specifically binds to GITR (e.g., human GITR), comprises a comprising a heavy chain variable region (VH) comprising:
(a) a VH-CDR1 comprising the amino acid sequence of X₁YAMX₂ (SEQ ID NO:1), wherein X₁ is D, G, or E; and X₂ is Y or H;
(b) a VH-CDR2 comprising the amino acid sequence of X₁IRTYSGX₂VX₃YNQKFX₄X₅ (SEQ ID NO: 2), wherein X₁ is V or L; X₂ is D or G; X₃ is T or S; X₄ is K, R, or Q; and X₅ is D, E, or G;
(c) a VH-CDR3 comprising the amino acid sequence of SGTVRGFAY (SEQ ID NO: 3); as shown in Table 5.

TABLE 4
GITR VL CDR amino acid sequences*

| Antibody | CL CDR1 (SEQ ID NO:) | VL CDR2 (SEQ ID NO:) | VL CDR3 (SEQ ID NO:) |
|---|---|---|---|
| Consensus 1 | KSSQSLLNSX₁NQKNYLX₂, | WASTRES (5) | QNX₁YSX₂PYT, |
| | wherein X₁ is G or S; | | wherein X₁ is D or |
| | and X₂ is T or S (90) | | E; and X₂ is Y, F, or |
| | | | S (92) |
| Consensus 2 | KSSQSLLNSX₁NQKNYLT | WASTRES (5) | QNX₁YSX₂PYT |
| | X₁ is G or S (4) | | X₁ is D or E; and |
| | | | X₂ is Y or F (6) |
| pab1876w | KSSQSLLNSGNQKNYLT (14) | WASTRES (5) | QNDYSYPYT (16) |
| pab1967w | KSSQSLLNSSNQKNYLT (15) | WASTRES (5) | QNEYSFPYT (17) |
| pab1975w | KSSQSLLNSGNQKNYLT (14) | WASTRES (5) | QNDYSYPYT (16) |
| pab1979w | KSSQSLLNSGNQKNYLT (14) | WASTRES (5) | QNDYSYPYT (16) |

*The VH CDRs in Table 5 are determined according to Kabat.

TABLE 5
GITR VH CDR amino acid sequences*

| Antibody | VHCDR1 (SEQ ID NO:) | VH CDR2 (SEQ ID NO:) | VH CDR3 (SEQ ID NO:) |
|---|---|---|---|
| Consensus 1 | X₁YX₂MX₃ | X₁IX₂TX₃SGX₄X₅X₆YNQKFX₇X₈, | SGTVRGFAY (3) |
| | wherein X₁ is D, | wherein X₁ is V or L; | |
| | E, or G; X₂ is A or | X₂ is R, K or Q; X₃ is | |
| | V; and X₃ is Y or H | Y or F; X₄ is D, E or G; | |
| | (87) | X₅ is V or L; X₆ is T or | |
| | | S; X₇ is K, R or Q; and | |
| | | X₈ is D, E or G (88) | |
| Consensus 2 | X₁YAMX₂ | X₁IRTYSGX₂VX₃YNQKFX₄X₅ | SGTVRGFAY (3) |
| | X₁ is D, G, or E; | X₁ is V or L; X₂ is D or | |
| | and X₂ is Y or H (1) | G; X₃ is T or S; X₄ is K, | |
| | | R, or Q; and X₅ is D, E, | |
| | | or G (2) | |
| pab1876w | DYAMY (7) | VIRTYSGDVTYNQKFKD (10) | SGTVRGFAY (3) |
| pab1967w | GYAMH (8) | LIRTYSGGVSYNQKFRE (11) | SGTVRGFAY (3) |
| pab1975w | EYAMH (9) | LIRTYSGGVSYNQKFQG (12) | SGTVRGFAY (3) |
| pab1979w | EYAMH (9) | VIRTYSGGVSYNQKFQE (13) | SGTVRGFAY (3) |

*The VH CDRs in Table 5 are determined according to Kabat.

In certain embodiments, provided herein is an antigen-binding domain which specifically binds to GITR (e.g., human GITR) and comprises light chain variable region (VL) CDRs and heavy chain variable region (VH) CDRs of pab1876w, pab1967w, pab1975w, or pab1979w, for example as set forth in Tables 1 and 2 (i.e., SEQ ID NOs: 14, 5, 16, 7, 10, and 3; SEQ ID NOs: 15, 5, 17, 8, 11, and 3; SEQ ID NOs: 14, 5, 16, 9, 12, and 3; or SEQ ID NOs: 14, 5, 16, 9, 13, and 3).

In certain embodiments, a GITR antigen-binding domain comprises a light chain variable framework region that is derived from human IGKV4-1 germline sequence (e.g., IGKV4-1*01, e.g., having the amino acid sequence of SEQ ID NO:28).

In certain embodiments, the GITR antigen-binding domain comprises a heavy chain variable framework region that is derived from a human IGHV1-2 germline sequence (e.g., IGHV1-2*02, e.g., having the amino acid sequence of SEQ NO:27).

In a specific embodiment, an antigen-binding domain that specifically binds to GITR (e.g., human GITR) comprises a VL domain comprising the amino acid sequence of SEQ ID NO: 19, 21, 23, or 26. In a specific embodiment, an antigen-binding domain that specifically binds to GITR (e.g., human GITR) comprises a VL domain consisting of or consisting essentially of the amino acid sequence of SEQ ID NO: 19, 21, 23, or 26.

In certain embodiments, an antigen-binding domain that specifically binds to GITR (e.g., human GITR) comprises a VH domain comprising the amino acid sequence of SEQ ID NO: 18, 20, 22, 24, or 25. In some embodiments, an antigen-binding domain that specifically binds to GITR (e.g., human GITR) comprises a VH domain consisting of or consisting essentially of the amino acid sequence of SEQ ID NO: 18, 20, 22, 24, or 25.

In certain embodiments, an antigen-binding domain that specifically binds to GITR (e.g., human GITR) comprises a VH domain and a VL domain, wherein the VH domain and the VL domain comprise the amino acid sequences of SEQ ID NOs:18 and 19; SEQ ID NOs:20 and 21; SEQ ID NOs:22 and 23; SEQ ID NOs:24 and 23; or SEQ ID NOs:25 and 26; respectively. In certain embodiments, an antigen-binding domain that specifically binds to GITR (e.g., human GITR) comprises a VH domain and a VL domain, wherein the VH domain and the VL domain consist of or consist essentially of the amino acid sequences of SEQ ID NOs:18 and 19; SEQ ID NOs:20 and 21; SEQ ID NOs:22 and 23; SEQ ID NOs:24 and 23; or SEQ ID NOs:25 and 26; respectively.

In specific aspects, provided herein is an antigen-binding domain comprising a light chain and heavy chain, e.g., a separate light chain and heavy chain. With respect to the light chain, in a specific embodiment, the light chain of an antigen-binding domain described herein is a kappa light chain. In another specific embodiment, the light chain of an antigen-binding domain described herein is a lambda light chain. In yet another specific embodiment, the light chain of an antigen-binding domain described herein is a human kappa light chain or a human lambda light chain. In a particular embodiment, an antigen-binding domain described herein, which immunospecifically binds to an GITR polypeptide (e.g., human GITR) comprises a light chain wherein the amino acid sequence of the VL domain comprises the sequence set forth in SEQ ID NO:19, 21, 23, or 26 and wherein the constant region of the light chain comprises the amino acid sequence of a human kappa light chain constant region. In another particular embodiment, an antigen-binding domain described herein, which immunospecifically binds to GITR (e.g., human GITR) comprises a light chain wherein the amino acid sequence of the VL domain comprises the sequence set forth in SEQ NO:19, 21, 23, or 26 and wherein the constant region of the light chain comprises the amino acid sequence of a human lambda light chain constant region in a specific embodiment, an antigen-binding domain described herein, which immunospecifically binds to GITR (e.g., human GITR) comprises a light chain wherein the amino acid sequence of the VL domain comprises the sequence set forth in SEQ ID NO:19, 21, 23, or 26 and wherein the constant region of the light chain comprises the amino acid sequence of a human kappa or lambda light chain constant region. Non-limiting examples of human constant region sequences have been described in the art, e.g., see U.S. Pat. No. 5,693,780 and Rabat, E A et al., (1991) supra.

With respect to the heavy chain, in a specific embodiment, the heavy chain of an antigen-binding domain described herein can be an alpha (α), delta (δ), epsilon (ε), gamma (γ) or mu (μ) heavy chain. In another specific embodiment, the heavy chain of an antigen-binding domain described can comprise a human alpha (α), delta (δ), epsilon (ε), gamma (γ) or mu (μ) heavy chain. In a particular embodiment, an antigen-binding domain described herein, which immunospecifically binds to GITR (e.g., human GITR ), comprises a heavy chain wherein the amino acid sequence of the VH domain can comprise the sequence set forth in SEQ ID NO:18 and wherein the constant region of the heavy chain comprises the amino acid sequence of a human gamma (γ) heavy chain constant region. In a specific embodiment, an antigen-binding domain described herein, which specifically binds to GITR (e.g., human GITR ), comprises a heavy chain wherein the amino acid sequence of the VH domain comprises the sequence set forth in SEQ ID NO:18, and wherein the constant region of the heavy chain comprises the amino acid of a human heavy chain described herein or known in the art. Non-limiting examples of human constant region sequences have been described in the art, e.g., see U.S. Pat. No. 5,693,780 and Kabat E A et al., (1991) supra. In a particular embodiment, an antigen-binding domain described herein, which specifically binds to GITR (e.g., human GITR), comprises a heavy chain comprising the amino acid sequence set forth in SEQ ID NO:29. In another embodiment, an antigen-binding domain described herein, which specifically binds to GITR (e.g., human GITR), comprises a heavy chain comprising the amino acid sequence set forth in SEQ ID NO:30. In another embodiment, an antigen-binding domain described herein, which specifically binds to GITR (e.g., human GITR), comprises a heavy chain comprising the amino acid sequence set forth in SEQ ID NO:36.

In a specific embodiment, an antigen-binding domain described herein, which immunospecifically binds to GITR (e.g., human GITR) comprises a VL domain and a VH domain comprising any amino acid sequences described herein, wherein the constant regions comprise the amino acid sequences of the constant regions of an IgG, IgE IgM, IgD, IgA, or IgY immunoglobulin molecule, or a human IgG, IgE, IgM, IgD, IgA, or IgY immunoglobulin molecule. In another specific embodiment, an antigen-binding domain described herein, which immunospecifically binds to GITR (e.g., human GITR) comprises a VL domain and a VH domain comprising any amino acid sequences described herein, wherein the constant regions comprise the amino acid sequences of the constant regions of an IgG, IgE, IgM, IgD, IgA, or IgY immunoglobulin molecule, any class (e.g., IgG₁, IgG₂, IgG₃, IgG₄, IgA₁, and IgA₂), or any subclass (e.g., IgG₂ₐ and IgG_{2b}) of immunoglobulin molecule. In a particular embodiment, the constant regions comprise the amino acid sequences of the constant regions of a human IgG, IgE, IgM, IgD, IgA, or IgY immunoglobulin molecule, any class (e.g., IgG₁, IgG₂, IgG₃, IgG₄, IgA₁, and IgA₂), or any subclass (e.g., IgG₂ₐ and IgG_{2b}) of immunoglobulin molecule.

In another specific embodiment, an antigen-binding domain described herein, which immunospecifically binds to GITR (e.g., human GITR), comprises a VL domain and a VH domain comprising any amino acid sequences described herein, wherein the constant regions comprise the amino acid sequences of the constant regions of a human IgG₁ (e.g., allotypes Glm3, Glm17,1 or Glm17,1,2), human IgG₂, or human IgG₄. In a particular embodiment, an antigen-binding domain described herein, which immunospecifically binds to GITR (e.g., human GITR), comprises a VL domain and a VH domain comprising any amino acid sequences described herein, wherein the constant regions comprise the amino acid sequences of the constant region of a human IgG₁ (allotype Glm3). Non-limiting examples of human constant regions are described in the art, e.g., see Kabat, E A et al., (1991) supra.

In certain embodiments, an antigen-binding domain described herein, which immunospecifically binds to GITR (e.g., human GITR), comprises a VL domain having at least 70%, at least 75%, at least 80%, at least 85, at least 90%, at least 95%, or at least 98% sequence identity to the amino acid sequence of the VL domain of pab1876w, pab1967w, pab1975w, or pab1979w (i.e., SEQ ID NO:19, 21, or 23), e.g., wherein the antigen-binding domain comprises VL CDRs that are identical to the VL CDRs of pab 1876w, pab1967w, pab1975w, or pab1979w.

In certain embodiments, an antigen-binding domain described herein, which immunospecifically binds to GITR (e.g., human GITR), comprises a VH domain having at least 70%, at least 75%, at least 80%, at least 85%, at least 90%, at least 95%, or at least 98% sequence identity to the amino acid sequence of the VH domain of pab1876w, pab19671,v, pab1975w, or pab1979w (i.e., SEQ ID INO:18, 20, 22, or 24), e.g., wherein the antigen-binding domain comprises VH CDRs that are identical to the VH CDRs of pab1876w, pab1967w, pab1975w, or pab1979w.

In certain embodiments, an antigen-binding domain described herein, which immunospecifically binds to GITR (e.g., human GITR), comprises: (i) a VL domain having at least 70%, at least 75%, at least 80%, at least 85%, at least 90%, at least 95%, or at least 98% sequence identity to the amino acid sequence of the VL domain of pab1876w, pab1967w, pab1975w, or pab1979w (i.e., SEQ ID NO:19, 21, or 23),; and (ii) a VH domain having at least 70%, at least 75%, at least 80%, at least 85%, at least 90%, at least 95%, or at least 98% sequence identity to the amino acid sequence of the VH domain of pab1876w, pab1967w, pab1975w, or pab1979w SEQ ID NO:18, 20, 22, or 24), e.g., wherein the antibody comprises VL CDRs and VH CDRs that are identical to the VL CDRs and VH CDRs of pab1876w, pab1967w, pab1975w, or pab1979w.

In certain aspects, an antigen-binding domain described herein may be described by its VL domain alone, by its VH domain alone, or by its 3 VL CDRs alone, or its 3 VH CDRs alone. See, for example, Rader, C el ral., PNAS 95: 8910-8915 (1998), which is incorporated herein by reference in its entirety, describing the humanization of the mouse anti-αvβ3 antibody by identifying a complementing light chain or heavy chain, respectively, from a human light chain or heavy chain library, resulting in humanized antibody variants having affinities as high or higher than the affinity of the original antibody. See also Clackson, T et al., Nature 352: 624-628 (1991), which is incorporated herein by reference in its entirety, describing methods of producing antibodies that bind a specific antigen by using a specific VL domain (or VH domain) and screening a library for the complementary variable domains. The screen produced 14 new partners for a specific VH domain and 13 new partners for a specific VL domain, which were strong binders, as determined by ELISA. See also Kim, S J & Hong, H J, J Microbiol 45: 572-577 (2007), which is incorporated herein by reference in its entirety, describing methods of producing antibodies that bind a specific antigen by using a specific VH domain and screening a library (e.g., human VL library) for complementary VL domains; the selected VL domains in turn could be used to guide selection of additional complementary (e.g., human) VH domains.

In certain aspects, the CDRs of an antigen-binding domain can be determined according to the Chothia numbering scheme, which refers to the location of immunoglobulin structural loops (see, e.g., Chothia, C & Lesk, A M, J Mol Biol 196: 901-917 (1987); Al-Lazikani, B et al., J Mol Biol 273: 927-948 (1997); Chothia, C et al., J Mol Biol 227: 799-817 (1992); Tramontano, A et al., J Mol Biol 215:175-82 (1990); and U.S. Pat. No. 7,709,226). Typically, when using the Kabat numbering convention, the Chothia CDR4I1 loop is present at heavy chain amino acids 26 to 32, 33, or 34, the Chothia CDR-H2 loop is present at heavy chain amino acids 52 to 56, and the Chothia CDR-H3 loop is present at heavy chain amino acids 95 to 102, while the Chothia CDR-L1 loop is present at light chain amino acids 24 to 34, the Chothia CDR-L2 loop is present at light chain amino acids 50 to 56, and the Chothia CDR-L3 loop is present at light chain amino acids 89 to 97. The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34).

In certain aspects, provided herein are antigen-binding domains that specifically bind to GITR (e.g., human GITR) and comprise the Chothia VL CDRs of a VL of pab1876w, pab1967w, pab1975w, or pab1979w. In certain aspects, provided herein are antigen-binding domains that specifically bind to GITR (e.g., human GITR) and comprise the Chothia CDRs of a VH of pab1876w, pab1967w, pab1975w, or pab1979w. In certain aspects, provided herein are antigen-binding domains that specifically bind to GITR (e.g., human GITR) and comprise the Chothia VL CDRs of a VL of pab1876w, pab1967w, pab1975w, or pab1979w and comprise the Chothia VH CDRs of a VH of pab1876w, pab1967w, pab1975w, or pab1979w in certain embodiments, antigen-binding domains that specifically bind to GITR (e.g., human GITR) comprise one or more CDRs, in which the Chothia and Kabat CDRs have the same amino acid sequence. In certain embodiments, provided herein are antigen-binding domains that specifically bind to GITR (e.g., human GITR) and comprise combinations of Kabat CDRs and Chothia CDRs.

In certain aspects, the CDRs of an antigen-binding domain can be determined according to the IMGT numbering system as described in Lefranc, M-P, The Immunologist 7: 132-136 (1999) and Lefranc, M-P et al., Nucleic Acids Res 27: 209-212 (1999). According to the IMGT numbering scheme, VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97. In a particular embodiment, provided herein are antigen-binding domains that specifically bind to GITR (e.g., human GITR) and comprise CDRs of pab1876w, pab1967w, pab1975w, pab1979w as determined by the IMGT numbering system, for example, as described in Lefranc, M-P (1999) supra and Lefranc, M-P et al. (1999) supra).

In certain aspects, the CDRs of an antigen-binding domain can he determined according to MacCallum, R M et al., J Mol Biol 262: 732-745 (1996). See also, e.g., Martin A. “Protein Sequence and Structure Analysis of Antibody Variable Domains,” in Antibody Engineering, Kontermann and Dubel, eds., Chapter 31, pp. 422-439, Springer-Verlag, Berlin (2001). In a particular embodiment, provided herein are antigen-binding domains that specifically bind to GITR (e.g., human GITR) and comprise CDRs of pab1876w, pab1967w, pab1975w, or pab1979w, as determined by the method in MacCallum, R M et al.

In certain aspects, the CDRs of an antibody can be determined according to the AbM numbering scheme, which refers AbM hypervariable regions which represent a compromise between the Kabat CDRs and Chothia structural loops, and are used by Oxford Molecular's AbM antibody modeling software (Oxford Molecular Group, Inc.). In a particular embodiment, provided herein are antigen-binding domains that specifically bind to GITR (e.g., human GITR) and comprise CDRs of pab1876w, pab1967w, pab1975w, or pab1979w as determined by the AbM numbering scheme.

In a specific embodiment, the position of one or more CDRs along the VH (e.g., CDR1, CDR2, or CDR3) and/or VL (e.g., CDR1, CDR2, or CDR3) region of an antigen-binding domain described herein may vary by one, two, three, four, five, or six amino acid positions so long as immunospecific binding to GITR (e.g., human GITR) is maintained (e.g., substantially maintained, for example, at least 50%, at least 60%, at least 70%, at least 80%, at least 90%, at least 95%). For example, in one embodiment, the position defining a CDR of an antigen-binding domain described herein can vary by shifting the N-terminal and/or C-terminal boundary of the CDR by one, two, three, four, five, or six amino acids, relative to the CDR position of an antigen-binding domain described herein, so long as immunospecific binding to GITR (e.g., human GITR) is maintained (e.g., substantially maintained, for example, at least 50%, at least 60%, at least 70%, at least 80%, at least 90%, at least 95%). In another embodiment, the length of one or more CDRs along the VH (e.g., CDR1, CDR2, or CDR3) and/or VT (e.g., CDR1, CDR2, or CDR3) region of an antigen-binding domain described herein may vary (e.g., be shorter or longer) by one, two, three, four, five, or more amino acids, so long as immunospecific binding to GITR (e.g., human GITR) is maintained (e.g., substantially maintained, for example, at least 50%, at least 60%, at least 70%, at least 80%, at least 90%, at least 95%).

In one embodiment, a VL CDR1, VL CDR2, VL CDR3, VH CDR1, VH CDR2, and/or VH CDR3 described herein may be one, two, three, four, five or more amino acids shorter than one or more of the CDRs described herein (e.g., SEQ ID NOs:1-6, SEQ ID NOs: 87, 88, 3, 90, 5, and 92; SEQ ID NOS: 7, 10, 3, 14, 5, and 16; SEQ ID NOs: 8, 11, 3, 15, 5, and 17; SEQ ID NOs: 9, 12, 3, 14, 5, and 16; or SEQ ID NOs: 9, 13, 3, 14, 5, and 16) so long as immunospecific binding to GITR (e.g., human GITR) is maintained (e.g., substantially maintained, for example, at least 50%, at least 60%, at least 70%, at least 80%, at least 90%, at least 95%). In another embodiment, a VL CDR1, VL CDR2, VL CDR3, VH CDR1, VH CDR2, and/or VH CDR3 described herein may be one, two, three, four, five or more amino acids longer than one or more of the CDRs described herein (e.g., SEQ ID NOs:1-6, SEQ ID NOs: 87, 88, 3, 90, 5, and 92; SEQ ID NOS: 7, 10, 3, 14, 5, and 16; SEQ ID NOs: 8, 11, 3, 15, 5, and 17; SEQ ID NOs: 9, 12, 3, 14, 5, and 16; or SEQ ID NOs: 9, 13, 3, 14, 5, and 16) so long as immunospecific binding to GITR (e.g., human GITR) is maintained (e.g., substantially maintained, for example, at least 50%, at least 60%, at least 70%, at least 80%, at least 90%, at least 95%). In another embodiment, the amino terminus of a VL CDR1, VL CDR2, VL CDR3, VH CDR1, VH CDR2, and/or VH CDR3 described herein may be extended by one, two, three, four, five or more amino acids compared to one or more of the CDRs described herein (e.g., SEQ ID NOs:1-6, SEQ ID NOs: 87, 88, 3, 90, 5, and 92; SEQ ID NOS: 7, 10, 3, 14, 5, and 16; SEQ ID NOs: 8, 11, 3, 15, 5, and 17; SEQ ID NOs: 9, 12, 3, 14, 5, and 16; or SEQ TD NOs: 9, 13, 3, 14, 5, and 16) so long as immunospecific binding to GITR (e.g., human GITR) is maintained (e.g., substantially maintained, for example, at least 50° x©, at least 60%, at least 70%, at least 80%, at least 90%, at least 95%). In another embodiment, the carboxy terminus of a VL CDR1, VL CDR2, VL CDR3, VH CDR1, VH CDR2, and/or VH CDR3 described herein may be extended by one, two, three, four, five or more amino acids compared to one or more of the CDRs described herein (e.g., SEQ ID NO:1-6) so long as immunospecific binding to GITR (e.g., human GITR) is maintained (e.g., substantially maintained, for example, at least 50%, at least 60%, at least 70%, at least 80%, at least 90%, at least 95%). in another embodiment, the amino terminus of a VL CDR1, VL CDR2, VL CDR3, CDR1, CDR2, and/or VH CDR3 described herein may be shortened by one, two, three, four, five or more amino acids compared to one or more of the CDRs described herein (e.g., SEQ ID NO:1-6) so long as immunospecific binding to GITR (e.g., human GITR) is maintained (e.g., substantially maintained, for example, at least 50%, at least 60%, at least 70%, at least 80%, at least 90%, at least 95%). In one embodiment, the carboxy terminus of a VL CDR1, VL CDR2, VL CDR3, VH CDR1, VH CDR2, and/or VH CDR3 described herein may be shortened by one, two, three, four, five or more amino acids compared to one or more of the CDRs described herein (e.g., SEQ ID NOs:1-6, SEQ ID NOs: 87, 88, 3, 90, 5, and 92; SEQ ID NOS: 7, 10, 3, 14, 5, and 16; SEQ ID NOs: 8, 11, 3, 15, 5, and 17; SEQ ID NOs: 9, 12, 3, 14, 5, and 16; or SEQ ID NOs: 9, 13, 3, 14, 5, and 16) so long as immunospecific binding to GITR (e.g., human GITR) is maintained (e.g., substantially maintained, for example, at least 50%, at least 60%, at least 70%, at least 80%, at least 90%, at least 95%). Any method known in the art can be used to ascertain whether immunospecific binding to GITR (e.g., human GITR) is maintained, for example, the binding assays and conditions described in the “Examples” section (Section 8) provided herein.

In another particular embodiment, an antigen-binding domain described herein, which immunospecifically binds to GITR (e.g., human GITR), comprises a heavy chain and a light chain, wherein (i) the heavy and light chains comprise a VH domain and a VL domain, respectively, wherein the VH CDR1, VH CDR2, VH CDR3, VL CDR1, VL CDR2, and VL CDR3 of the VH and VL domains comprise the amino acid sequences set forth in SEQ ID NOs:1-6, SEQ ID NOs: 87, 88, 3, 90, 5, and 92; SEQ ID NOS: 7, 10, 3, 14, 5, and 16; SEQ ID NOs: 8, 11, 3, 15, 5, and 17; SEQ ID NOs: 9, 12, 3, 14, 5, and 16; or SEQ ID NOs: 9, 13, 3, 14, 5, and 16, respectively; (ii) the light chain further comprises a constant light chain domain comprising the amino acid sequence of the constant domain of a human kappa light chain; and (iii) the heavy chain further comprises a constant heavy chain domain comprising the amino acid sequence of the constant domain of a human IgG₁ (optionally IgG₁ (allotype Glm3)) heavy chain.

In another particular embodiment, an antigen-binding domain described herein, which immunospecifically binds to GITR (e.g., human GITR), comprises a heavy chain and a light chain, wherein (i) the heavy and light chains comprise a VH domain and a VL, domain, respectively comprising the amino acid sequences set forth in SEQ ID NOs: 18 and 19, SEQ ID NOs: 20 and 21, SEQ ID NOs: 22 and 23, SEQ ID NOs: 24 and 23, or SEQ ID NOs: 25 and 26, respectively; (ii) the light chain further comprises a constant domain comprising the amino acid sequence of the constant domain of a human kappa light chain; and (iii) the heavy chain further comprises a constant domain comprising the amino acid sequence of the constant domain of a human IgG₁ (optionally IgG₁ (allotype Glm3)) heavy chain.

In another particular embodiment, an antigen-binding domain described herein, which immunospecifically hinds to GITR (e.g., human GITR), comprises a light chain and a heavy chain, wherein (i) the heavy and light chains comprise a VH domain and a VL domain, respectively, wherein the VH CDR1, VH CDR2, VH CDR3, VL CDR1, VL CDR2, and VL CDR3 of the VH and VL domains comprise the amino acid sequences set forth in SEQ ID NOs:1-6, SEQ ID NOs: 87, 88, 3, 90, 5, and 92; SEQ ID NOS: 7, 10, 3, 14, 5, and 16; SEQ ID NOs: 8, 11, 3, 15, 5, and 17; SEQ ID NOs: 9, 12, 3, 14, 5, and 16; or SEQ ID NOs: 9, 13, 3, 14, 5, and 16, respectively; (ii) the light chain further comprises a constant light chain domain comprising the amino acid sequence of the constant domain of a human kappa light chain; and (iii) the heavy chain further comprises a constant heavy chain domain comprising the amino acid sequence of the constant domain of a human IgG₄ heavy chain.

In another particular embodiment, an antigen-binding domain described herein, which immunospecifically binds to GITR (e.g., human GITR), comprises a light chain and a heavy chain, wherein (i) the heavy and light chains comprise a VH domain and a VL domain, respectively comprising the amino acid sequences set forth in SEQ ID NOs: 18 and 19, SEQ ID NOs: 20 and 21, SEQ ID NOs: 22 and 23, SEQ ID NOs: 24 and 23, or SEQ ID NOs: 25 and 26, respectively; (ii) the light chain further comprises a constant domain comprising the amino acid sequence of the constant domain of a human kappa light chain; and (Hi) the heavy chain further comprises a constant domain comprising the amino acid sequence of the constant domain of a human IgG₄ heavy chain.

In another particular embodiment, an antigen-binding domain described herein, which immunospecifically binds to GITR (e.g., human GITR), comprises a light chain and a heavy chain, wherein (i) the heavy and light chains comprise a VH domain and a VL domain, respectively, wherein the VH CDR1, VH CDR2, VH CDR3, VL CDR1, VL CDR2, and VL CDR3 of the VH and VL domains comprise the amino acid sequences set forth in SEQ ID NOs:1-6, SEQ ID NOs: 87, 88, 3, 90, 5, and 92; SEQ ID NOS: 7, 10, 3, 14, 5, and 16; SEQ ID NOs: 8, 11, 3, 15, 5, and 17; SEQ ID NOs: 9, 12, 3, 14, 5, and 16; or SEQ ID NOs: 9, 13, 3, 14, 5, and 16, respectively; (ii) the light chain further comprises a constant light chain domain comprising the amino acid sequence of the constant domain of a human kappa light chain; and (iii) the heavy chain further comprises a constant heavy chain domain comprising the amino acid sequence of the constant domain of a human IgG₂ heavy chain.

In another particular embodiment, an antibody described herein, which immunospecifically binds to GITR (e.g., human GITR), comprises a light chain and a heavy chain, wherein (i) the heavy and light chains comprise a VH domain and a VL domain, respectively comprising the amino acid sequences set forth in SEQ ID NOs: 18 and 19, SEQ ID NOs: 20 and 21, SEQ ID NOs: 22 and 23, SEQ ID NOs: 24 and 23, or SEQ ID NOs: 25 and 26, respectively; (ii) the light chain further comprises a constant domain comprising the amino acid sequence of the constant domain of a human kappa light chain; and (iii) the heavy chain further comprises a constant domain comprising the amino acid sequence of the constant domain of a human IgG₂ heavy chain.

In another specific embodiment, an antibody provided herein, which specifically binds to GITR (e.g., human GITR), comprises (a) a heavy chain comprising the amino acid sequence of SEQ NO:29 with an amino acid substitution of N to A or Q at amino acid position 297, numbered according to the EU numbering system; and (b) a light chain comprising the amino acid sequence of SEQ ID NO:37.

In another specific embodiment, an antibody provided herein, which specifically binds to GITR (e.g., human GITR), comprises (a) a heavy chain comprising the amino acid sequence of SEQ ID NO:29 with an amino acid substitution selected from the group consisting of: S to E at amino acid position 267, L to F at amino acid position 328, and both S to E at amino acid position 267 and L to F at amino acid position 328, numbered according to the EU numbering system; and (b) a light chain comprising the amino acid sequence of SEQ ED NO:37.

In specific embodiments, an antigen-binding domain described herein, which immunospecifically binds to GITR (e.g., human GITR), comprises framework regions (e.g., framework regions of the VL domain and/or VH domain) that are human framework regions or derived from human framework regions. Non-limiting examples of human framework regions are described in the art, e.g., see Kabat, E A et al., (1991) supra). In certain embodiment, an antigen-binding domain described herein comprises framework regions (e.g., framework regions of the VL domain and/or VH domain) that are primate(e.g., non-human primate) framework regions or derived from primate (e.g., non-human primate) framework regions.

For example, CDRs from antigen-specific non-human antibodies, typically of rodent origin (e.g., mouse or rat), are grafted onto homologous human or non-human primate acceptor frameworks. In one embodiment, the non-human primate acceptor frameworks are from Old World apes. In a specific embodiment, the Old World ape acceptor framework is from Pan troglodytes, Pan paniscus or Gorilla gorilla. In a particular embodiment, the non-human primate acceptor frameworks are from the chimpanzee Pan troglodytes. In a particular embodiment, the non-human primate acceptor frameworks are Old World monkey acceptor frameworks. In a specific embodiment, the Old World monkey acceptor frameworks are from the genus Macaca. In a certain embodiment, the non-human primate acceptor frameworks are derived from the cynomolgus monkey Macaca cynomolgus. Non-human primate framework sequences are described in U.S. Patent Application Publication No. US 2005/0208625.

In another aspect, provided herein are antibodies that contain antigen-binding domains that bind the same or an overlapping epitope of GITR (e.g., an epitope of human GITR) as an antibody described herein (e.g., pab1876w). In certain embodiments, the epitope of an antibody can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giegé, R et al., (1994) Acta Crystallogr D Biol Crystallogr 50(Pt 4): 339-350; McPherson, A (1990) Eur J Biochem 189: 1-23; Chayen, N E (1997) Structure 5: 1269-1274; McPherson, A (1976) J Biol Chem 251: 6300-6303). Antibody:antigen crystals may be studied using well known X-ray diffraction techniques and may be refined using computer software such as X-PLOR (Yale University, 1992, distributed by Molecular Simulations, Inc.; see, e.g., Meth Enzymol (1985) volumes 114 & 115, eds Wyckoff, H W et al.; U.S. Patent Application No. 2004/0014194), and BUSTER (Bricogne, G (1993) Acta Crystallogr D Biol Crystallogr 49(Pt 1): 37-60; Bricogne, G (1997) Meth Enzymol 276A: 361-423, ed. Carter, C W; Roversi, P et al., (2000) Acta Crystallogr D Biol. Crystallogr 56(Pt 10): 1316-1323). Mutagenesis mapping studies may be accomplished using any method known to one of skill in the art. See, e.g., Champe, M et al., (1995) supra and Cunningham, B C & Wells, J A (1989) supra for a description of mutagenesis techniques, including alanine scanning mutagenesis techniques. In a specific embodiment, the epitope of an antigen-binding domain is determined using alanine scanning mutagenesis studies. In addition, antigen-binding domains that recognize and bind to the same or overlapping epitopes of GITR (e.g., human) can be identified using routine techniques such as an immunoassay, for example, by showing the ability of one antibody to block the binding of another antibody to a target antigen, i.e., a competitive binding assay. Competition binding assays also can be used to determine whether two antibodies have similar binding specificity for an epitope. Competitive binding can be determined in an assay in which the immunoglobulin under test inhibits specific binding of a reference antibody to a common antigen, such as GITR. Numerous types of competitive binding assays are known, for example: solid phase direct or indirect radioimmunoassay (RIA), solid phase direct or indirect enzyme immunoassay (EIA,), sandwich competition assay (see Stahli, C et al., (1983) Methods Enzymol 9: 242-253); solid phase direct biotin-avidin EIA (see Kirkland, T N et al., (1986) J Immunol 137: 3614-9); solid phase direct labeled assay, solid phase direct labeled sandwich assay (see Harlow, E & Lane, D, (1988) Antibodies: A Laboratory Manual, Cold Spring Harbor Press); solid phase direct label RIA using 1-125 label (see Morel, G A et al., (1988) Mol Immunol 25(1): 7-15); solid phase direct biotin-avidin EIA (Cheung, R C et al., (1990) Virology 176: 546-52); and direct labeled RIA. (Moldenhauer, G et al., Scand J Immunol 32: 77-82 (1990)). Typically, such an assay involves the use of purified antigen (e.g., GITR, such as human GITR) bound to a solid surface or cells bearing either of these, an unlabeled test immunoglobulin and a labeled reference immunoglobulin. Competitive inhibition can be measured by determining the amount of label bound to the solid surface or cells in the presence of the test immunoglobulin. Usually the test immunoglobulin is present in excess. Usually, when a competing antibody is present in excess, it will inhibit specific binding of a reference antibody to a common antigen by at least 50-55%, 55-60%, 60-65%, 65-70%, 70-75% or more. A competition binding assay can be configured in a large number of different formats using either labeled antigen or labeled antibody. In a common version of this assay, the antigen is immobilized on a 96-well plate. The ability of unlabeled antibodies to block the binding of labeled antibodies to the antigen is then measured using radioactive or enzyme labels. For further details see, for example, Wagener, C et al., (1983) J Immunol 130: 2308-2315; Wagener, C et al., (1984) J Immunol Methods 68: 269-274; Kuroki, M et al., (1990) Cancer Res 50: 4872-4879; Kuroki, M et al., (1992) Immunol Invest 21: 523-538; Kuroki, M et al., (1992) Hybridoma 11: 391-407 and Antibodies: A Laboratory Manual, Ed Harlow E & Lane D editors supra, pp. 386-389.

In one embodiment, a competition assay is performed using surface plasmon resonance (BIAcore®), e.g., by an ‘in tandem approach’ such as that described by Abdiche, Y N et al., (2009) Analytical Biochem 386: 172-180, whereby GITR antigen is immobilized on the chip surface, for example, a CM5 sensor chip and the anti-GITR antibodies are then run over the chip. To determine if an antibody competes with an anti-GITR antigen-binding domain described herein, the antibody containing the anti-GITR antigen-binding domain is first run over the chip surface to achieve saturation and then the potential, competing antibody is added. Binding of the competing antibody can then be determined and quantified relative to a non-competing control.

In certain aspects, competition binding assays can be used to determine whether an antibody is competitively blocked, e.g., in a dose dependent manner, by another antibody for example, an antibody binds essentially the same epitope, or overlapping epitopes, as a reference antibody, when the two antibodies recognize identical or sterically overlapping epitopes in competition binding assays such as competition ELISA assays, which can be configured in all number of different formats, using either labeled antigen or labeled antibody. In a particular embodiment, an antibody can be tested in competition binding assays with an antibody described herein (e.g., antibody pab1876w), or a chimeric or Fab antibody thereof, or an antibody comprising VH CDRs and VL CDRs of an antibody described herein (e pab1876w).

In another aspect, provided herein are antigen-binding domains that compete (e.g., in a dose dependent manner) for binding to GITR (e.g., human GITR) with an antigen-binding domain described herein, as determined using assays known to one of skill in the art or described herein (e.g., ELISA competitive assays or surface plasmon resonance). In another aspect, provided herein are antigen-binding domains that competitively inhibit (e.g., in a dose dependent manner) an antigen-binding domain described herein (e.g., pab1876w) from binding to GITR (e.g., human GITR), as determined using assays known to one of skill in the art or described herein (e.g., ELISA competitive assays, or suspension array or surface plasmon resonance assay). In specific aspects, provided herein is an antigen-binding fragment which competes (e.g., in a dose dependent manner) for specific binding to GITR (e.g., human GITR), with an antibody comprising the amino acid sequences described herein (e.g., VL and/or VH amino acid sequence of pab1876w), as determined using assays known to one of skill in the art or described herein (e.g., ELISA competitive assays, or suspension array or surface plasmon resonance assay).

In certain embodiments, provided herein is an antigen-binding domain that competes with an antigen-binding domain described herein for binding to GITR (e.g., human GITR) to the same extent that the antigen-binding fragment described herein self-competes for binding to GITR (e.g., human GITR). In some embodiments, provided herein is a first antigen-binding antibody domain that competes with an antigen-binding antibody domain described herein for binding to GITR (e.g., human GITR), wherein the first antigen-binding domain competes for binding in an assay comprising the following steps: (a) incubating GITR-transfected cells with the first antigen-binding domain in unlabeled form in a container; and (b) adding an antigen-binding domain described herein in labeled form in the container and incubating the cells in the container; and (c) detecting the binding of the antigen-binding domain described herein in labeled form to the cells. In certain embodiments, provided herein is a first antigen-binding domain that competes with an antigen-binding domain described herein for binding to GITR (e.g., human GITR), wherein the competition is exhibited as reduced binding of the first antigen-binding domain to GITR by more than 80% (e.g., 85%, 90%, 95%, or 98%, or between 80% to 85%, 80% to 90%, 85% to 90%, or 85% to 95%).

In specific aspects, provided herein is an antigen-binding domain which competes (e.g., in a dose dependent manner) for specific binding to GITR (e.g., human GITR), with an antigen-binding domain comprising a VH and VL domain having the amino acid sequences set forth in SEQ ID NOs:18 and 19; SEQ ID NOs: 20 and 21, SEQ ID NOs: 22 and 23 or SEQ ID NOs: 24 and 23, respectively.

In a specific embodiment, an antigen-binding domain described herein is one that is competitively blocked (e.g., in a dose dependent manner) by an antigen-binding domain comprising a VH and VL domain having the amino acid sequences set forth in SEQ ID NOs:18 and 19; SEQ ID NOs: 20 and 21, SEQ ID NOs: 22 and 23 or SEQ ID NOs: 24 and 23, respectively for specific binding to GITR (e.g., human GITR).

Assays known to one of skill in the art or described herein (e.g., X-ray crystallography, hydrogen/deuterium exchange coupled with mass spectrometry (e.g., liquid chromatography electrospray mass spectrometry), alanine scanning, ELISA assays, etc.) can be used to determine if two antibodies bind to the same epitope.

In a specific embodiment, an antigen-binding domain described herein immunospecifically binds to the same epitope as that bound by pab1876w, pab1967w, pab1975w, or pab1979w or an epitope that overlaps the epitope.

In a specific aspect, the binding between an antigen-binding domain described herein and a variant GITR is substantially weakened relative to the binding between the antigen-binding domain and a human GITR sequence of residues 26 to 241 of SEQ ID NO:41, wherein the variant GITR comprises the sequence of residues 26 to 241 of SEQ ID NO:41 except for the presence of a D60A or G63A mutation (e.g., substitution), numbered according to SEQ ID NO: 41. In some embodiments, the variant GITR comprises the sequence of residues 26 to 241 of SEQ ID NO:41 except for the presence of a D60A and a G63A mutation, numbered according to SEQ ID NO: 41.

In a specific aspect, an antigen-binding domain described herein binds to an epitope of a human GITR sequence comprising, consisting essentially of, or consisting of at least one residue in amino acids 60-63 of SEQ ID NO:41. In some embodiments, the epitope comprises, consists essentially of, or consists of amino acids 60-63 of SEQ ID NO:41.

In a specific embodiment, an antigen-binding domain described herein binds to an epitope of human GITR, comprising, consisting essentially of, or consisting of a residue selected from the group consisting of: residues 60, 62, and 63, and a combination thereof of SEQ ID NO:41. In some embodiments, the epitope comprises, consists essentially of, or consists of any one residue, or any two, or three residues, selected from the group consisting of: residues 60, 62, and 63 of SEQ ID NO:41.

In a specific aspect, an antigen-binding domain described herein exhibits, as compared to binding to a human GITR sequence of residues 26 to 241 of SEQ ID NO:41, reduced or absent binding to a protein identical to residues 26 to 241 of SEQ ID NO:41 except for the presence of an amino acid mutation (e.g., substitution) selected from the group consisting of: D60A and G63A, numbered according to SEQ ID NO: 41, In some embodiments, the substitution is D60A, numbered according to SEQ ID NO: 41. In some embodiments, the substitution is G63A, numbered according to SEQ NO: 41.

### 7.2.2 Constant Region Mutations and Modifications

In certain embodiments, one, two, or more mutations (e.g., amino acid substitutions) are introduced into the Fc region of an antibody described herein (e.g., CH2 domain (residues 231-340 of human IgG₁) and/or CH3 domain (residues 341-447 of human IgG₁) and/or the hinge region, with numbering according to the EU numbering system to alter one or more functional properties of the antibody, such as serum half-life, complement fixation, Fc receptor binding and/or antigen-dependent cellular cytotoxicity.

In certain embodiments, one, two, or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain may be altered to, e.g., facilitate assembly of the light and heavy chains, or to alter (e.g., increase or decrease) the stability of the antibody.

In some embodiments, one, two, or more mutations (e.g., amino acid substitutions) are introduced into the Fc region of an antibody described herein (e.g., CH2 domain (residues 231-340 of human IgG₁) and/or CH3 domain (residues 341-447 of human IgG₁) and/or the hinge region, with numbering according the EU numbering system to increase or decrease the affinity of the antibody for an Fc receptor (e.g., an activated Fe receptor) on the surface of an effector cell. Mutations in the Fe region of an antibody that decrease or increase the affinity of an antibody for an Fe receptor and techniques for introducing such mutations into the Fe receptor or fragment thereof are known to one of skill in the art. Examples of mutations in the Fe receptor of an antibody that can be made to alter the affinity of the antibody for an Fe receptor are described in, e.g., Smith, P et al., PNAS 109: 6181-6186 (2012), U.S. Pat. No. 6,737,056, and International. Publication Nos. WO 02/060919; WO 98/23289; and WO 97/34631, which are incorporated herein by reference.

In a specific embodiment, one, two, or more amino acid mutations substitutions, insertions or deletions) are introduced into an IgG constant domain, or FcRn-binding fragment thereof (preferably an Fe or hinge-Fe domain fragment) to alter (e.g., decrease or increase) half-life of the antibody in vivo. See e.g., International Publication Nos. WO02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo. In some embodiments, one, two or more amino acid mutations (i.e., substitutions, insertions, or deletions) are introduced into an IgG constant domain, or FcRn-binding fragment thereof (preferably an Fe or hinge-Fe domain fragment) to decrease the half-life of the antibody in vivo. In other embodiments, one, two or more amino acid mutations substitutions, insertions or deletions) are introduced into an IgG constant domain, or FcRn-binding fragment thereof (preferably an Fe or hinge-Fe domain fragment) to increase the half-life of the antibody in vivo. In a specific embodiment, the antibodies may have one or more amino acid mutations (e.g., substitutions) in the second constant (CH2) domain (residues 231-340 of human IgG₁) and/or the third constant (CH3) domain (residues 341-447 of human IgG₁), with numbering according to the EU numbering system. In a specific embodiment, the constant region of the IgG₁ of an antibody described herein comprises a methionine (M) to tyrosine (Y) substitution in position 252, a serine (S) to threonine (T) substitution in position 254, and a threonine (T) to glutatnic acid (E) substitution in position 256, numbered according to the EU numbering system. See U.S. Pat. No. 7,658,921, which is incorporated herein by reference. This type of mutant IgG, referred to as “YTE mutant” has been shown to display fourfold increased half-life as compared to wild-type versions of the same antibody (see Dall'Acqua, W F et al. J Biol Chem 281: 23514-24 (2006)). In certain embodiments, an antibody comprises an IgG constant domain comprising one, two, three or more amino acid substitutions of amino acid residues at positions 251-257, 285-290, 308-314, 385-389, and 428-436, numbered according to the EU numbering system.

In a further embodiment, one, two, or more amino acid substitutions are introduced into an IgG constant domain Fc region to alter the effector function(s) of the antibody. For example, one or more amino acids selected from amino acid residues 234, 235, 236, 237, 297, 318, 320 and 322, numbered according to the EU numbering system, can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260. In some embodiments, the deletion or inactivation (through point mutations or other means) of a constant region domain may reduce Fc receptor binding of the circulating antibody thereby increasing tumor localization. See, e.g., U.S. Pat. Nos. 5,585,097 and 8,591,886 for a description of mutations that delete or inactivate the constant domain and thereby increase tumor localization. In certain embodiments, one or more amino acid substitutions may be introduced into the Fc region of an antibody described herein to remove potential glycosylation sites on Fc region, which may reduce Fc receptor binding (see, e.g., Shields, R L et at., J Biol Chem 276: 6591-604 (2001)). In various embodiments, one or more of the following mutations in the constant region of an antibody described herein may be made: an N297A substitution; an N297Q_{.} substitution; or a D265A substitution, numbered according to the EU numbering system. In various embodiments, one or more of the following mutations in the constant region of an antibody described herein may be made: a D265A substitution, a P329A substitution, or a combination thereof, numbered according to the EU numbering system.

In a specific embodiment, an antibody described herein comprises the constant domain of an IgG₁ with an N297Q, N297A, or D265A amino acid substitution, or a combination thereof, numbered according to the EU numbering system. In a specific embodiment, an antibody described herein comprises the constant domain of an IgG₁ with a D265A or P329A amino acid substitution, or a combination thereof, numbered according to the EU numbering system.

In certain embodiments, one or more amino acids selected from amino acid residues 329, 331, and 322 in the constant region of an antibody described herein, numbered according to the EU numbering system, can be replaced with a different amino acid residue such that the antibody has altered C1q binding and/or reduced or abolished complement dependent cytotoxicity (CDC). This approach is described in further detail in U.S. Pat. No. 6,194,551 (Idusogie et al). In some embodiments, one or more amino acid residues within amino acid positions 231 to 238, numbered according to the EU numbering system, in the N-terminal region of the CH2 domain of an antibody described herein are altered to thereby alter the ability of the antibody to fix complement. This approach is described further in International Publication No. WO 94/29351. In certain embodiments, the Fc region of an antibody described herein is modified to increase the ability of the antibody to mediate antibody dependent cellular cytotoxicity (ADCC) and/or to increase the affinity of the antibody for an Fcγ receptor by mutating one or more amino acids (e.g., introducing amino acid substitutions) at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 328, 329, 330, 331, 333, 334, 335, 337, 338, 340, 360, 373, 376, 378, 382, 388, 389, 398, 414, 416, 419, 430, 434, 435, 437,438, or 439, numbered according to the EU numbering system. This approach is described further in International Publication No. WO 00/42072.

In certain embodiments, an antibody described herein comprises the constant domain of an IgG₁ with a mutation (e.g., substitution) at position 267, 328, or a combination thereof, numbered according to the EU numbering system. In certain embodiments, an antibody described herein comprises the constant domain of an IgG₁ with a mutation (e.g., substitution) selected from the group consisting of S267E, L328F, and a combination thereof, numbered according to the EU numbering system. In certain embodiments, an antibody described herein comprises the constant domain of an IgG₁ with a S267E/L328F mutation (e.g., substitution), numbered according to the EU numbering system. In certain embodiments, an antibody described herein comprising the constant domain of an IgG₁ with a S267E/L3281; mutation (e.g., substitution) has an increased binding affinity for FcγRIIA, FcγRIIB, or FcγRIIA and FcγRIIB, numbered according to the EU numbering system.

In certain embodiments, an antibody described herein comprises the constant region of an IgG₄ antibody and the serine at amino acid residue 228 of the heavy chain, numbered according to the EU numbering system, is substituted for proline.

In certain embodiments, an antibody described herein comprises the constant region of an IgG, antibody and the cysteine at amino acid residue 127 of the heavy chain, numbered according to Kabat, is substituted for serine.

Antibodies with reduced fucose content have been reported to have an increased affinity for Fc receptors, such as, e.g., FcγRIIIa. Accordingly, in certain embodiments, the antibodies described herein have reduced fucose content or no fucose content. Such antibodies can be produced using techniques known to one skilled in the art. For example, the antibodies can be expressed in cells deficient or lacking the ability of fucosylation. In a specific example, cell lines with a knockout of both alleles of α1,6-fucosyltransferase can be used to produce antibodies with reduced fucose content. The Potelligent® system (Lonza) is an example of such a system that can be used to produce antibodies with reduced fucose content. Alternatively, antibodies with reduced fucose content or no fucose content can be produced by, e.g.: (i) culturing cells under conditions which prevent or reduce fucosylation; (ii) posttranslational removal of fucose (e.g., with a fucosidase enzyme); (iii) post-translational addition of the desired carbohydrate, e.g., after recombinant expression of a non-glycosylated glycoprotein; or (iv) purification of the glycoprotein so as to select for antibodies thereof which are not fucsoylated. See, e.g., Longmore, G D & Schachter, H Carbohydr Res 100: 365-92 (1982) and Imai-Nishiya, H et al., BMC Biotechnol. 7: 84 (2007) for methods for producing antibodies thereof with no fucose content or reduced fucose content.

Engineered glycoforms may be useful for a variety of purposes, including but not limited to enhancing or reducing effector function. Methods for generating engineered glycoforms in an antibody described herein include but are not limited to those disclosed, e.g., in Umaña, P et al., Nat Biotechnol 17: 176-180 (1999); Davies, J et al., Biotechnol Bioeng 74: 288-294 (2001); Shields, R L et al., J Biol Chem 2:77: 26733-26740 (2002); Shinkawa, T et al., J Biol Chem 278: 3466-3473 (2003); Niwa, R et al., Clin Cancer Res 1: 6248-6255 (2004); Presta, L G et al., Biochem Soc Trans 30: 487-490 (2002); Kanda, Y et al., Glycobiology 17: 104-118 (2007); U.S. Pat. Nos. 6,602,684; 6,946,292; and 7,214,775; U.S. Patent Publication Nos. US 2007/0248600; 2007/0178551; 2008/0060092; and 2006/0253928; International Publication Nos. WO00/61739; ‘WO 01/292246; WO02/311140; and ‘WO 02/30954; Potillegent™ technology (Biowa, Inc. Princeton, N.J.); and GlycoMAb® glycosylation engineering technology (Glycart biotechnology AG, Zurich, Switzerland). See also, e.g., Ferrara, C et al., Biotechnol Bioeng 93: 851-861 (2006); international Publication Nos. WO 07/039818; WO 12/130831; WO 99/054342; WO03/011878; and WO 04/065540.

In certain embodiments, the technology used to engineer the Fc domain of an antibody described herein is the Xmab™ Technology of Xencor (Monrovia, Calif.). See, e.g., U.S. Pat. Nos. 8,367,805; 8,039,592; 8,124,731; 8,188,231; U.S. Patent Publication No. 2006/0235208; International Publication Nos. WO 05/077981; WO 11/097527; and Richards J O et al., (2008) Mol Cancer Ther 7: 2517-2527.

In certain embodiments, any of the constant region mutations or modifications described herein can be introduced into one or both heavy chain constant regions of an antibody described herein having two heavy chain constant regions.

### 7.23 Anti-GITR Antibodies

In a specific aspect, an antibody as described herein which immunospecifically binds to GITR (e.g., human GITR), comprises: (a) a first antigen-binding domain that specifically binds to GITR (e.g, human GITR), as described herein; and (b) a second antigen-binding domain that does not specifically bind to an antigen expressed by a human immune cell (i.e., the second antigen-binding domain does not specifically bind to GITR (e.g., human GITR) or any other antigen expressed by a human immune cell), as described herein. In certain embodiments, the antigen to which the second antigen-binding domain specifically binds is not naturally expressed by a human immune cell. In certain embodiments, the immune cell is selected from the group consisting of a T cell (e.g., a CD4+ T cell or a CD8+ T cell), a B cell, a natural killer cell, a dendritic cell, a macrophage, and an eosinophil. In certain embodiments, the antigen-binding domain that specifically binds to GITR (e.g., human GITR) comprises a first VH and a first VL, and the second antigen-binding domain comprises a second VH and a second VL. In certain embodiments, the antigen-binding domain that specifically binds to GITR (e.g., human GITR) comprises a first heavy chain and a first light chain, and the second antigen-binding domain comprises a second heavy chain and a second light chain. In certain embodiments, the antibody is for administration to a sample or subject in which the second antigen-binding domain is non-reactive (i.e., the antigen to which the second antigen-binding domain specifically binds is not present in the sample or subject). In certain embodiments, the second antigen-binding domain does not specifically bind to an antigen on a cell expressing GITR (e.g., human GITR), In certain embodiments, the second antigen-binding domain does not specifically bind to an antigen that is naturally expressed by a cell that expresses GITR (e.g., human GITR). In certain embodiments, the antibody functions as a monovalent antibody (i.e., an anti-GITR-monovalent antibody) in a sample or subject, wherein the first antigen-binding domain of the antibody specifically binds to GITR (e.g., human GITR), while the second antigen-binding domain is non-reactive in the sample or subject, for example, due to the absence of antigen to which the second antigen-binding domain binds in the sample or subject.

In certain embodiments, the second antigen-binding domain specifically binds to a non-human antigen (i.e., an antigen expressed in other organisms and not humans). In certain embodiments, the second antigen-binding domain specifically binds to a viral antigen. In certain embodiments, the viral antigen is from a virus that does not infect humans (i.e., a non-human virus). In certain embodiments, the viral antigen is absent in a human immune cell (e.g., the immune cell is uninfected with the virus associated with the viral antigen). In certain embodiments, the viral antigen is a HIV antigen. In certain embodiments, the second antigen-binding domain specifically binds to chicken albumin or hen egg lysozyme. In certain embodiments, the second antigen-binding domain specifically binds to an antigen that is not expressed by (i.e., is absent from) wild-type cells (e.g., wild-type human cells). In certain embodiments, the second antigen-binding domain specifically binds to a tumor-associated antigen that is not expressed by (i.e., is absent from) normal cells (e.g., wild-type cells, e.g., wild-type human cells). In certain embodiments, the tumor-associated antigen is not expressed by (i.e., is absent from) human cells. In certain embodiments, the second antigen-binding domain comprises a heavy chain comprising a mutation selected from the group consisting of: N297A, N297Q, D265A, S228P, and a combination thereof, numbered according to the EU numbering system. In certain embodiments, the mutation is N297A, N297Q, D265A, or a combination thereof, numbered according to the EU numbering system. In certain embodiments, the mutation is S228P, numbered according to the EU numbering system. In certain embodiments, the second antigen-binding domain comprises a heavy chain comprising a mutation selected from the group consisting of: D265A, P329A, and a combination thereof, numbered according to the EU numbering system. In certain embodiments, the second antigen-binding domain comprises a heavy chain comprising a C127S mutation, numbered according to Kabat.

In certain embodiments, the first antigen-binding domain comprises a first heavy chain and the second antigen-binding domain comprises a second heavy chain, wherein the heavy chains are selected from the group consisting of immunoglobulins IgG₁, IgG₂, IgG₃, IgG₄, IgA₁, and IgA₂. In certain embodiments, the immunoglobulins are human immunoglobulins. Human immunoglobulins containing mutations (e.g., substitutions) are also referred to as human immunoglobulins herein. In certain embodiments, first and second antigen-binding domains comprise heavy chains of the same isotype. When the first and second antigen-binding domains are the same isotype, the sequences associated with the second antigen-binding domain are also described herein as “isotype” sequences (e.g., isotype VH or isotype HC). In certain embodiments, the first antigen-binding domain comprises a first human IgG₁ heavy chain and the second antigen-binding domain comprises a second human IgG₁ heavy chain. In certain embodiments, the first antigen-binding domain comprises a first human IgG₁ heavy chain and the second antigen-binding domain comprises a second human IgG₁ heavy chain, wherein the first and second heavy chains comprise an identical mutation selected from the group consisting of N297A, N297Q, D265A, and a combination thereof, numbered according to the EU numbering system. In certain embodiments, the first antigen-binding domain comprises a first human IgG₁ heavy chain and the second antigen-binding domain comprises a second human IgG₁ heavy chain, wherein the first and second heavy chains comprise an identical mutation selected from the group consisting of D265A, P329A, and a combination thereof, numbered according to the EU numbering system. In certain embodiments, the first antigen-binding domain comprises a first human IgG₂ heavy chain and the second antigen-binding domain comprises a second human IgG₂ heavy chain, wherein the first and second heavy chains comprise a C127S mutation, numbered according Kabat. In certain embodiments, the first antigen-binding domain comprises a first human IgG₄ heavy chain and the second antigen-binding domain comprises a second human IgG₄ heavy chain, wherein the first and second heavy chains comprise a S228P mutation, numbered according to the EU numbering system. In certain embodiments, the antibody is antagonistic.

In another specific aspect, an antibody as described herein which immunospecifically binds to GITR (e.g., human GITR), comprises: (a) an antigen-binding domain that specifically binds to GITR (e.g., human GITR), as described herein, comprising a first heavy chain and a light chain; and (b) a second heavy chain or fragment thereof, as described herein. Such an antibody can optionally comprise a first light chain or fragment thereof and a second light chain or fragment thereof. The first light chain can comprise a first light chain constant domain and a first light chain variable domain. The second light chain can comprise a second light chain constant domain and a second light chain variable domain. In some embodiments, the fragment of the second heavy chain is an Fc fragment. In some embodiments, the heavy chain or second heavy chain comprises a constant domain and a variable domain. In certain embodiments, the second heavy chain or fragment thereof is from an antigen-binding domain that specifically binds to GITR (e.g., human GITR). In certain embodiments, the second heavy chain or fragment thereof is from an antigen-binding domain that specifically binds to a non-human antigen (i.e., an antigen expressed in other organisms and not humans). In certain embodiments, the second heavy chain or fragment thereof is from an antigen-binding domain that specifically binds to a viral antigen. In certain embodiments, the viral antigen is from a virus that does not infect humans (i.e., a non-human virus). In certain embodiments, the viral antigen is absent in an immune cell (e.g., the immune cell is uninfected with the virus associated with the viral antigen). In certain embodiments, the viral antigen is a HIV antigen. In certain embodiments, the second heavy chain or fragment thereof is from an antigen-binding domain that specifically binds to chicken albumin or hen egg lysozyme. In certain embodiments, the second heavy chain or fragment thereof is from an antigen-binding domain that specifically binds to an antigen that is not expressed by (i.e., is absent from) wild-type cells (e.g., wild-type human cells). In certain embodiments, the second heavy chain or fragment thereof is from an antigen-binding domain that specifically binds to a tumor-associated antigen that is not expressed by (i.e., is absent from) normal cells (e.g., wild-type cells, e.g., wild-type human cells). In certain embodiments, the tumor-associated antigen is not expressed by (i.e., is absent from) human cells. In certain embodiments, the second heavy chain or fragment thereof comprises a mutation selected from the group consisting of: N297A, N297Q, D265A, S228P, and a combination thereof, numbered according to the EU numbering system. In certain embodiments, the mutation is N297A, N297Q, D265A, or a combination thereof, numbered according to the EU numbering system. In certain embodiments, the mutation is S228P, numbered according to the EU numbering system. In certain embodiments, the second heavy chain or fragment thereof comprises a mutation selected from the group consisting of: D265A, P329A, and a combination thereof, numbered according to the EU numbering system. In certain embodiments, the second heavy chain or fragment thereof comprises a C127S mutation, numbered according to Kabat. In certain embodiments, the first heavy chain and the second heavy chain are selected from the group consisting of immunoglobulins IgG₁ IgG2, IgG₃, IgG₄, IgA₁, and IgA₂. In certain embodiments, the immunoglobulins are human immunoglobulins. In certain embodiments, first and second heavy chains are the same isotype. When the first and second heavy chains are the same isotype, the sequences associated with the second heavy chain are also described herein as “isotype” sequences (e.g., isotype VH or isotype HC). In certain embodiments, the first and second heavy chains are IgG₁ heavy chains. In certain embodiments, the first and second heavy chains are IgG₁ heavy chains, wherein the first and second heavy chains comprise an identical mutation selected from the group consisting of N297A, N297Q, D265A, and a combination thereof, numbered according to the EU numbering system. In certain embodiments, the first and second heavy chains are IgG₁ heavy chains, wherein the first and second heavy chains comprise an identical mutation selected from the group consisting of D265A, P329A, and a combination thereof, numbered according to the EU numbering system. In certain embodiments, the first and second heavy chains are IgG₂ heavy chains, wherein the first and second heavy chains comprise a C127S mutation, numbered according to Kabat. In certain embodiments, the first and second heavy chains are IgG₄ heavy chains, wherein the first and second heavy chains comprise a S2281P mutation, numbered according to the EU numbering system, in certain embodiments, the antibody is antagonistic.

In the above aspects, the first and second antigen-binding domains or the first and second heavy chains can comprise complementary CH3 domains. For example, the complementary CH3 domains allow for heterodimerization to preferentially occur between two different antigen-binding domains or two different heavy chains, rather than homodimerization between the same antigen-binding domains or the same heavy chains. Any technique known to those of skill in the art can be used to produce complementary CH3 domains, including, but not limited to, knob-into-hole technology as described in Ridgway, J B B et al., Protein Eng 9(7): 617-621 (1996) and Merchant, M et al. For example, the knob-into-hole technology replaces a small amino acid with a larger amino acid (i.e., the “knob”) in a first CH3 domain and replaces a large amino acid with a smaller amino acid (i.e., the “hole”) in a second CH3 domain. Polypeptides comprising the CH3 domains can then dimerize based on interaction of the knob and hole. In certain embodiments that include a first antigen-binding domain and a second antigen-binding domain, one of the antigen-binding domains chains comprises a first IgG₁ CH3 domain comprising a substitution selected from the group consisting of 1366Y and T366W, and the other antigen-binding domain comprises a second IgG₁ CH3 domain comprising a substitution selected from the group consisting of Y407T, T366S, L368A, Y407V, numbered according to the EU numbering system. In certain embodiments that include a first heavy chain and a second heavy chain, one of the heavy chains comprises a first IgG₁ CH3 domain comprising a substitution selected from the group consisting of T366Y and T366W, and the other heavy chain comprises a second IgG₁ CH3 domain comprising a substitution selected from the group consisting of Y407T, T366S, L368A, Y407V, numbered according to the EU numbering system.

In a specific aspect, an antibody which immunospecifically binds to GITR (e.g., human GITR), comprises an antigen-binding domain that specifically binds to GITR (e.g., human GITR) as described herein (i.e., a heavy chain variable region sequence and a light chain variable region sequence of an antigen-binding domain that specifically hinds to GITR (e.g., human GITR) as described herein), wherein the antibody is selected from the group consisting of a Fab, Fab′, F(ab′)₂, and scFv fragment, A Fab, Fab′, F(ab′)₂, or scFv fragment can be produced by any technique known to those of skill in the art, including, but not limited to, those discussed in Section 7.3, infra. In certain embodiments, the Fab, Fab′, F(ab′)₂, or scFv fragment further comprises a moiety that extends the half-life of the fragment in vivo. The moiety is also termed a “half-life extending moiety.” Any moiety known to those of skill in the art for extending the half-life of an antibody fragment in vivo can he used. For example, the half-life extending moiety can include an Fc region, a polymer, an albumin, or an albumin binding protein or compound. The polymer can include a natural or synthetic, optionally substituted straight or branched chain polyalkylene, polyalkenylene, polyoxylalkylene, polysaccharide, polyethylene glycol, polypropylene glycol, polyvinyl alcohol, methoxypolyethylene glycol, lactose, amylose, dextran, glycogen, or derivative thereof. Substituents can include one or more hydroxy, methyl, or methoxy groups. In certain embodiments, the antibody fragment can be modified by the addition of one or more C-terminal amino acids for attachment of the half-life extending moiety. In certain embodiments the half-life extending moiety is polyethylene glycol or human serum albumin. In certain embodiments, the Fab, Fab′, F(ab′)₂, or scFv fragment is fused to an Fc region. In certain embodiments, the antibody is antagonistic.

In a specific aspect, an antibody which immunospecifically binds to GITR (e.g., human GITR), comprises one antigen-binding domain that specifically binds to GITR (e.g., human GITR) as described herein, wherein the antigen-binding domain comprises one heavy chain and one light chain as described herein (i.e., the antibody does not comprise any additional heavy chain or light chain and only contains a single heavy chain-light chain pair). In certain embodiments, the heavy chain is selected from the group consisting of immunoglobulins IgG₁, IgG₂, IgG₃, IgG₄, IgA₁, and IgA₂. In certain embodiments, the immunoglobulins are human immunoglobulins. In certain embodiments, the heavy chain comprises a mutation selected from the group consisting of: N297A, N297Q, D265A, S228P, and a combination thereof, numbered according to the EU numbering system. In certain embodiments, the mutation is N297A, N297Q, D265A, or a combination thereof, numbered according to the EU numbering system. In certain embodiments, the mutation is S228P, numbered according to the EU numbering system. In certain embodiments, the heavy chain comprises a C127S mutation, numbered according to Kabat. In certain embodiments, the heavy chain comprises a mutation selected from the group consisting of: D265A, P329A, and a combination thereof, numbered according to the EU numbering system. In certain embodiments, the heavy chain is an IgG₁ heavy chain comprising a mutation selected from the group consisting of N297A, N297Q, D265A, and a combination thereof, numbered according to the EU numbering system. In certain embodiments, the heavy chain is an IgG₁ heavy chain comprising a mutation selected from the group consisting of D265A, P329A, and a combination thereof, numbered according to the EU numbering system. In certain embodiments, the heavy chain is an IgG₂ heavy chain comprising a C127S mutation, numbered according to Kabat. In certain embodiments, the heavy chain is an IgG₄ heavy chain comprising a S228P mutation, numbered according to the EU numbering system. In certain embodiments, the antibody is antagonistic.

In the above aspects directed to an antibody comprising an antigen-binding domain that specifically binds to GITR (e.g., human GITR) and either a second antigen-binding domain or a second heavy chain or fragment thereof, the antigen-binding domain can comprise any of the anti-GITR sequences described herein. In certain embodiments, the antigen-binding domain that specifically binds to GITR (e.g., human GITR) comprises: (a) a first heavy chain variable domain (VH) comprising a VH-complementarity determining region (CDR) 1 comprising the amino acid sequence of X₁YX₂MX₃ (SEQ ID NO:87), wherein X₁ is D, E or G; X₂ is A or V, and X₃ is Y or H; a VH-CDR2 comprising the amino acid sequence of X₁IX₂TX₃SGX₄X₅X₆YNQKFX₇X₈(SEQ ID NO:88), wherein X₁ is V or L, X₂ is R, K or Q, X₃ is Y or F, X₄ is D, E or G, X₅ is V or L, X₆ is T or S, X₇ is K, R or Q, and X₈ is D, E or G; and a VH-CDR3 comprising the amino acid sequence of SGTVRGFAY (SEQ ID NO:3); and (b) a first light chain variable domain (VL) comprising a VL-CDR1 comprising the amino acid sequence of KSSQSLLNSX₁NQKNYLX₂(SEQ ID NO:90), wherein X₁ is G or S, and X₂ is T or S; a VL-CDR2 comprising the amino acid sequence of WASTRES (SEQ ID NO:5); and a VL-CDR3 comprising the amino acid sequence of QNX₁YSX₂PYT (SEQ ID NO:92), wherein X₁ is D or E; and X₂ is Y, F or S. In certain embodiments, the antigen-binding domain that specifically binds to GITR (e.g., human GITR) binds to the same epitope of GITR (e.g., human GITR) as an antibody comprising a VH comprising the amino acid sequence of SEQ ID NO:18 and a VL comprising the amino acid sequence of SEQ ID NO:19. In certain embodiments, the antigen-binding domain that specifically binds to GITR (e.g., human GITR) exhibits, as compared to binding to a human GITR sequence of residues 26 to 241 of SEQ ID NO:41, reduced or absent binding to a protein identical to residues 26 to 241 of SEQ ID NO:41 except for the presence of a D60A or G63A amino acid substitution, numbered according to SEQ ID NO: 41. In certain embodiments, the antigen-binding domain that specifically binds to (e.g., human GITR) comprises a VH and a VL, wherein the VH comprises the amino acid sequence selected from the group consisting of SEQ ID NOs: 18, 20, 22, 24, and 25. In certain embodiments, the antigen-binding domain that specifically binds to (e.g., human GITR) comprises a VH and a VL, wherein the VL comprises the amino acid sequence selected from the group consisting of SEQ ID NOs: 19, 21, 23, and 26. In certain embodiments, the antigen-binding domain that specifically binds to GITR (e.g., human (GITR) specifically binds to an epitope of GITR (e.g., human GITR) comprising at least one amino acid in residues 60-63 of SEQ ID NO:41. In certain embodiments, the antigen-binding domain that binds to GITR (e.g., human GITR) specifically binds to each of i) human GITR, comprising amino acid residues 26 to 241 of SEQ ID NO:41; and ii) a variant of cynomolgus GITR, said variant comprising amino acid residues 26-234 of SEQ ID NO:46, wherein the antigen-binding domain that specifically binds to human GITR does not specifically bind to cynomolgus GITR comprising amino acid residues 26-234 of SEQ ID NO:44. In certain embodiments, the antigen-binding domain that specifically binds to GITR (e.g., human GITR) comprises a VH-CDR1, comprising an amino acid sequence selected from the group consisting of SEQ ID NOs: 7-9. In certain embodiments, the antigen-binding domain that specifically binds to GITR (e.g., human GITR) comprises a VH-CDR2 comprising an amino acid sequence selected from the group consisting of SEQ ID NOs: 10-13. In certain embodiments, the antigen-binding domain that specifically binds to GITR (e.g., human GITR) comprises a VL-CDR1 comprising the amino acid sequence of SEQ ID NO: 14 or 15. In certain embodiments, the antigen-binding domain that specifically binds to GITR (e.g., human GITR) comprises a VL-CDR3 comprising the amino acid sequence of SEQ ID NO: 16 or 17. In certain embodiments, the antigen-binding domain that specifically binds to GITR (e.g., human GITR) comprises VH-CDR1, VH-CDR2, and VH-CDR3 sequences set forth in SEQ ID NOs: 7, 10, and 3; SEQ ID NOs: 8, 11, and 3; SEQ ID NOs: 9, 12, and 3; or SEQ ID NOs: 9, 13, and 3, respectively; and/or VL-CDR1, VL-CDR2, and VL-CDR3 sequences set forth in SEQ ID NOs: 14, 5, and 16; or SEQ ID NOs: 15, 5, and 17, respectively. In certain embodiments, the antigen-binding domain that specifically binds to GITR (e.g., human GITR) comprises VH-CDR1, VH-CDR2, VH-CDR3, VL-CDR1, VL-CDR2, and VL-CDR3 sequences set forth in SEQ ID NOs: 7, 10, 3, 14, 5, and 16, respectively. In certain embodiments, the antigen-binding domain that specifically binds to GITR (e.g., human GITR) comprises a VH comprising the sequence set forth in SEQ ID NO:25. In certain embodiments, the antigen-binding domain that specifically binds to GITR (e.g., human GITR) comprises a VH comprising an amino acid sequence at least 75%, 80%, 85?, 90%, 95%, or 99% identical to an amino acid sequence selected from the group consisting of SEQ ID NOs: 18, 20, 22, and 24. In certain embodiments, the antigen-binding domain that specifically binds to GITR (e.g., human GITR) comprises a VH comprising an amino acid sequence selected from the group consisting of SEQ ID NOs: 18, 20, 22, and 24. In certain embodiments, the antigen-binding domain that specifically binds to GITR (e.g., human GITR) comprises a VH comprising the amino acid sequence of SEQ ID NO:18. In certain embodiments, the antigen-binding domain that specifically binds to GITR (e.g., human GITR) comprises a heavy chain comprising the amino acid sequence of SEQ ID NOs: 29, 30, or 36. In certain embodiments, the antigen-binding domain that specifically binds to GITR (e.g., human GITR) comprises a VH comprising an amino acid sequence derived from a human IGHV1-2 germline sequence (e.g., IGHV1-2*02, e.g., having the amino acid sequence of SEQ ID NO:27). In certain embodiments, the antigen-binding domain that specifically binds to GITR (e.g., human GITR) comprises a VL comprising the amino acid sequence of SEQ ID NO: 26. In certain embodiments, the antigen-binding domain that specifically binds to GITR (e.g., human GITR) comprises a VL comprising an amino acid sequence at least 75%, 80%, 85%, 90%, 95?, or 99% identical to an amino acid sequence selected from the group consisting of SEQ ID NOs: 19, 21, and 23. In certain embodiments, the antigen-binding domain that specifically binds to GITR (e.g., human GITR) comprises a VL comprising an amino acid sequence selected from the group consisting of SEQ ID NOs: 19, 21, and 23. In certain embodiments, the antigen-binding domain that specifically binds to GITR (e.g., human GITR) comprises a VL comprising the amino acid sequence of SEQ ID NO:19. In certain embodiments, the antigen-binding domain that specifically binds to GITR (e.g., human GITR) comprises a light chain comprising the amino acid sequence of SEQ ID NO: 37. In certain embodiments, the antigen-binding domain that specifically binds to GITR (e.g., human GITR) comprises a light chain comprising the amino acid sequence of SEQ ID NO: 38. In certain embodiments, the antigen-binding domain that specifically binds to GITR (e.g., human GITR) comprises a VL comprising an amino acid sequence derived from a human IGKV4-1 germline sequence (e.g., IGKV4-1*01, e.g., having the amino acid sequence of SEQ ID NO:28). In certain embodiments, the antigen-binding domain that specifically binds to GITR (e.g., human GITR) comprises VH and VL sequences set forth in SEQ ID NOs: 18 and 19, SEQ ID NOs: 20 and 21, SEQ ID NOs: 22 and 23, or SEQ ID NOs: 2.4 and 23, respectively. In certain embodiments, the antigen-binding domain that specifically binds to GITR (e.g., human GITR) comprises In certain embodiments, the antigen-binding domain that specifically binds to GITR (e.g., human GITR) comprises a VH comprising the sequence set forth in SEQ ID NO:18 and a VL comprising the sequence set forth in SEQ ID NO:19, In certain embodiments, the antigen-binding domain that specifically binds to GITR (e.g., human GITR) comprises a heavy chain selected from the group consisting of immunoglobulins IgG₁, IgG₂, IgG₃, IgG₄, IgA₁, and IgA₂. In certain embodiments, the immunoglobulins are human immunoglobulins. In certain embodiments, the heavy chain is an IgG₁ heavy chain comprising a mutation selected from the group consisting of N297A, N297Q, D265A, and a combination thereof, numbered according to the EU numbering system. In certain embodiments, the heavy chain is an IgG₁ heavy chain comprising a mutation selected from the group consisting of D265A, P329A, and a combination thereof, numbered according to the EU numbering system. In certain embodiments, the heavy chain is an IgG₂ heavy chain comprising a C127S mutation, numbered according to Kabat. In certain embodiments, the heavy chain is an IgG₄ heavy chain comprising a S228P mutation, numbered according to the EU numbering system.

In certain embodiments, an antagonistic antibody described herein is antagonistic to GITR (e.g., human GITR). In certain embodiments, the antibody deactivates, reduces, or inhibits an activity of GITR (e.g., human GITR). In certain embodiments, the antibody inhibits or reduces binding of GITR (e.g., human GITR) to GITR ligand (e.g., human GITR ligand). In certain embodiments, the antibody inhibits or reduces GITR (e.g., human GITR) signaling. In certain embodiments, the antibody inhibits or reduces GITR (e.g., human GITR) activity (e.g., GITR signaling) induced by GITR ligand (e.g., human GITR ligand). In certain embodiments, an antagonistic antibody described herein inhibits or reduces T cell proliferation. In certain embodiments, an antagonistic antibody described herein inhibits or reduces production of cytokines (e.g., inhibits or reduces production of IL-2, TNFα, IFNγ, IL-4, IL-10, IL-13, or a combination thereof by stimulated T cells). In certain embodiments, an antagonistic antibody described herein inhibits or reduces production of IL-2 by SEA-stimulated T cells. In certain embodiments, an antagonistic antibody described herein blocks the interaction of GPM and GITRL (e.g., blocks the binding of GITRL and GITR to one another, e.g., blocks the binding of human GITR ligand and human GITR)).

In certain embodiments, an antagonistic antibody described herein, which immunospecifically binds to GITR (e.g., human GITR) decreases GITR (e.g., human GITR) activity by at least about 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold as assessed by methods described herein and/or known to one of skill in the art, relative to GITR (e.g., human GITR) activity without any antibody or with an unrelated antibody (e.g., an antibody that does not immunospecifically bind to GITR). In certain embodiments, an antagonistic antibody described herein, which immunospecifically binds to GITR (e.g., human GITR), decreases GITR (e.g., human GITR) activity by at least 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein and/or known to one of skill in the art, relative to GITR (e.g., human GITR) activity without any antibody or with an unrelated antibody (e.g., an antibody that does not immunospecifically bind to GITR). Non-limiting examples of GITR (e.g., human GITR) activity can include GITR (e.g., human GITR) signaling, cell proliferation, cell survival, and cytokine production (e.g., TFN-α, IFN-γ, IL-4, IL-10, and/or IL-13). In certain embodiments, an antagonistic antibody described herein, which itnmunospecifically binds to GITR (e.g., human GITR), inhibits, reduces, or inactivates an GITR (e.g., human GITR) activity. In specific embodiments, GITR activity is assessed as described in the Examples, infra.

In certain aspects, an antagonistic antibody described herein, which immunospecifically binds to GITR (e.g., human GITR), inhibits, reduces, or deactivates the cellular proliferation of cells that express GITR and that respond to GITR signaling (e.g., cells that proliferate in response to GITR stimulation and GITR signaling, such as T cells). Cell proliferation assays are described in the art, such as a ³H-thymidine incorporation assay, BrdU incorporation assay, or CFSE assay, and can be readily carried out by one of skill in the art. In specific embodiments, T cells (e.g., CD4⁺ or CD8⁺ effector T cells) stimulated with a T cell mitogen or T cell receptor complex stimulating agent (e.g., phytohaemagglutinin (PHA) and/or phorbol myristate acetate (PMA), or a TCR complex stimulating antibody, such as an anti-CD3 antibody and anti-CD28 antibody), in the presence of an antagonistic antibody described herein, which immunospecifically binds to GITR (e.g., human GITR), have decreased cellular proliferation relative to T cells only stimulated with the T cell mitogen or T cell receptor complex stimulating agent, such as phytohaemagglutinin (PHA) and/or phorbol myristate acetate (PMA), or a TCR complex stimulating antibody, such as an anti-CD3 antibody and anti-CD28 antibody.

In certain aspects, an antagonistic antibody described herein, which immunospecifically binds to GITR (e.g., human GITR), decreases the survival of cells (e.g., T cells, such as CD4 and CD8 effector T cells). In a specific embodiment, T cells (e.g., CD4⁺ or CD8⁺ effector T cells) stimulated with a T cell mitogen or T cell receptor complex stimulating agent (e.g., phytohaemagglutinin (pHA) and/or phorbol myristate acetate (PMA), or a TCR complex stimulating antibody, such as an anti-CD3 antibody and anti-CD28 antibody) in the presence of an antagonistic antibody described herein, which immunospecifically binds to GITR (e.g., human GITR), have decreased survival relative to T cells only stimulated with the T cell mitogen. Cell survival assays are described in the art (e.g., a trypan blue exclusion assay) and can be readily carried out by one of skill in the art.

In specific embodiments, an antagonistic antibody described herein, which immunospecifically binds to GITR (e.g., human GITR), decreases cell survival (e.g., T cells, such as CD4 and CD8 effector T cells) by at least about 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold, as assessed by methods described herein or known to one of skill in the art (e.g., a trypan blue exclusion assay), without any antibody or with an unrelated antibody (e.g., an antibody that does not immunospecifically bind to GITR). In specific embodiments, an antagonistic antibody described herein, which immunospecifically binds to GITR (e.g., human GITR), decreases cell survival (e.g., T cells, such as CD4 and CD8 effector T by at least about 5%, 10%, 15%, 20%, 25%, 30%, 35%, 40%, 45%, 50%, 55%, 60%, 65%, 70%, 75%, 80%, 85%, 90%, 95%, 98%, or 99%, as assessed by methods described herein or known to one of skill in the art (e.g., a trypan blue exclusion assay), relative to GITR (e.g., human MR) activity without any antibody or with an unrelated antibody (e.g., an antibody that does not immunospecifically bind to GITR).

In some embodiments, T cells (e.g., CD4⁺ or CD8⁺ effector T cells) stimulated with a T cell mitogen (e.g., an anti-CD3 antibody or phorbol ester) in the presence of an antagonistic antibody described herein, which immunospecifically binds to MR (e.g., human GITR), have decreased cell survival by at least about 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold relative to T cells only stimulated with the T cell mitogen or cell receptor complex stimulating agent (e.g., phytohaemagglutinin (PHA) and/or phorbol myristate acetate (PMA), or a TCR complex stimulating antibody, such as an anti-CD3 antibody and anti-CD28 antibody), as assessed by methods described herein or known to one of skill in the art (e.g., a trypan blue exclusion assay). In some embodiments, T cells (e.g., CD4⁺ or CD8⁺ effector T cells) stimulated with a T cell mitogen or cell receptor complex stimulating agent (e.g., phytohaemagglutinin (PHA) and/or phorbol mytistate acetate (PMA), or a TCR complex stimulating antibody, such as an anti-CD3 antibody and anti-CD28 antibody) in the presence of an antagonistic antibody described herein, which immunospecifically binds to GITR (e.g., human MR), have decreased cell survival by at least about 5%, 10%, 15%, 20%, 25%, 30%, 35%, 40%, 45%, 50%, 60%, 65%, 70%, 75%, 80%, 85%, 90%, 95%, 98%, or 99% relative to T cells only stimulated with the T cell mitogen, as assessed by methods described herein or known to one of skill in the art (e.g., a trypan blue exclusion assay).

In certain embodiments, an antagonistic antibody described herein, which immunospecifically binds to GITR (e.g., human GITR), does not protect effector T cells (e.g., CD4⁺ and CD8⁺ effector T cells) from activation-induced cell death.

In specific embodiments, an antagonistic antibody described herein, which immunospecifically binds to GITR (e.g., human GITR), inhibits, reduces, or deactivates cytokine production (e.g., IL-2, TNF-α, IFN-γ, IL-4, IL-10, and/or IL-13) by at least about 5%, 10%, 15%, 20%, 25%, 30%, 35%, 40%, 45%, 50%, 55%, 60%, 65%, 70%, 75%, 80%, 85%, 90%, 95%, 98%, or 99%, as assessed by methods described herein or known to one of skill in the art, relative to cytokine production in the presence or absence of GITRL (e,g, human GITRL) stimulation without any antibody or with an unrelated antibody (e.g., an antibody that does not immunospecifically bind to GITR). In specific embodiments, an antagonistic antibody described. herein, which immunospecifically binds to GITR (e.g., human GITR), inhibits or reduces cytokine production (e.g., IL-2, TNF-α, IFN-γ, IL-4, IL-10, and/or IL-13) by at least about 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold, as assessed by methods described herein or known to one of skill in the art, relative to cytokine production in the presence or absence of GITRL (e.g., human GITRL) stimulation without any antibody or with an unrelated antibody (e.g., an antibody that does not immunospecifically bind to GITR).

In certain embodiments, T cells (e.g., CD4⁺ or CD8⁺ effector T cells) stimulated with a T cell mitogen or T cell receptor complex stimulating agent (e.g., phytohaema.gglutinin (PHA) and/or phorbol myristate acetate (PMA), or a TCR complex stimulating antibody, such as an anti-CD3 antibody and anti-CD28 antibody) in the presence of an antagonistic antibody described herein, which immunospecifically binds to GITR (e.g., human GITR), have decreased cytokine production (e.g., IL-2, TNF-α, IFN-γ, IL-4, IL-10, and/or IL-13) by at least about 5%, 10%, 15%, 20%, 25%, 30%, 35%, 40%, 45%, 50%, 55%, 60%, 65%, 70%, 75%, 80%, 85%, 90%, 95%, 98%, or 99% relative to T cells only stimulated with the T cell mitogen or T cell receptor complex stimulating agent (e.g., phytohaemagglutinin (PHA) and/or phorbol myristate acetate (PMA), or a TCR complex stimulating antibody, such as an anti-CD3 antibody and anti-CD28 antibody), as assessed by methods described herein or known to one of skill in the art (e.g., an ELISA assay). In some embodiments, T cells (e.g., CD4⁺ or CD8⁺ effector T cells) stimulated with a T cell mitogen or T cell receptor complex stimulating agent (e.g., phytohaemagglutinin (PHA) and/or phorbol myristate acetate (PMA), or a TCR complex stimulating antibody, such as an anti-CD3 antibody and anti-CD28 antibody) in the presence of an antagonistic antibody described herein, which immunospecifically binds to GITR (e.g., human GITR), have decreased cytokine production (e.g., IL-2, TNF-α, INF-γ, IL-4, IL-10, and/or IL-13) by at least about 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold relative to T cells only stimulated with the T cell mitogen or T cell receptor complex stimulating agent (e.g., phytohaemagglutinin (PHA) and/or phorbol myristate acetate (PMA), or a TCR complex stimulating antibody, such as an anti-CD3 antibody and anti-CD28 antibody), as assessed by methods described herein or known to one of skill in the art (e.g., an ELISA assay).

An anti-GITR antibody can be fused or conjugated (e.g., covalently or noncovalently linked) to a detectable label or substance. Examples of detectable labels or substances include enzyme labels, such as, glucose oxidase; radioisotopes, such as iodine (¹²⁵I, ¹²¹I), carbon (¹⁴C), sulfur ³⁵S), tritium (³H), indium (¹²¹In), and technetium (⁹⁹Tc); luminescent labels, such as luminal; and fluorescent labels, such as fluorescein and rhodamine, and biotin. Such labeled antibodies can be used to detect GITR (e.g., human GITR) protein. See, e.g., Section 7.5.2, infra.

### 7.3 Antibody Production

Antibodies that immunospecifically bind to GITR (e.g., human GITR) can be produced by any method known in the art for the synthesis of antibodies, for example, by chemical synthesis or by recombinant expression techniques. The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described, for example, in the references cited herein and are fully explained in the literature. See, e.g., Maniatis, T et al., (1982) Molecular Cloning: A Laboratory Manual, Cold Spring Harbor Laboratory Press; Sambrook, J et al., (1989), Molecular Cloning: A Laboratory Manual, Second Edition, Cold Spring Harbor Laboratory Press; Sambrook J et al., (2001) Molecular Cloning: A Laboratory Manual, Cold Spring Harbor Laboratory Press, Cold Spring Harbor, N.Y.; Ausubel, F M et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren, B et al., (eds.) (1999) Genome Analysis: A Laboratory Manual, Cold Spring Harbor Laboratory Press.

In a specific embodiment, an antibody described herein is an antibody (e.g., recombinant antibody) prepared, expressed, created or isolated by any means that involves creation, e.g., via synthesis, genetic engineering of DNA sequences. In certain embodiments, such antibody comprises sequences (e.g., DNA sequences or amino acid sequences) that do not naturally exist within the antibody germline repertoire of an animal or mammal (e.g., human) vivo.

In a certain aspect, provided herein is a method of making an antibody which immunospecifically binds to GITR (e.g., human GITR) comprising culturing a cell or host cell described herein. In a certain aspect, provided herein is a method of making an antibody which immunospecifically binds to GITR (e.g., human GITR) comprising expressing (e.g., recombinantly expressing) the antibody using a cell or host cell described herein (e.g., a cell or a host cell comprising polynucleotides encoding an antibody described herein). In a particular embodiment, the cell is an isolated cell. In a particular embodiment, the exogenous polynucleotides have been introduced into the cell. In a particular embodiment, the method further comprises the step of purifying the antibody obtained from the cell or host cell.

Methods for producing polyclonal antibodies are known in the art (see, for example, Chapter 11 in: Short Protocols in Molecular Biology, (2002) 5th Ed. Ausubel, F M et al., eds., John Wiley and Sons, New York).

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow, E & Lane, D, Antibodies: A Laboratory Manual, (Cold Spring Harbor Laboratory Press, 2nd ed. 1988); Hammerling, G J et al., in: Monoclonal Antibodies and I-Cell hybridomas 563 681 (Elsevier, N.Y., 1981). The term “monoclonal antibody” as used herein is not limited to antibodies produced through hybridoma technology. For example, monoclonal antibodies can be produced recombinantly from host cells exogenously expressing an antibody described herein.

In specific embodiments, a “monoclonal antibody,” as used herein, is an antibody produced by a single cell (e.g., hybridoma or host cell producing a recombinant antibody), wherein the antibody immunospecifically binds to GITR (e.g., human GITR) as determined, e.g., by ELISA or other antigen-binding or competitive binding assay known in the art or in the Examples provided herein. In particular embodiments, a monoclonal antibody can be a chimeric antibody or a humanized antibody. In certain embodiments, a monoclonal antibody is a monovalent antibody or multivalent (e.g., bivalent) antibody. In certain embodiments, a monoclonal antibody can be a Fab fragment or a F(ab′)₂ fragment. Monoclonal antibodies described herein can, for example, be made by the hybridoma method as described in Kohler, G & Milstein, C, Nature 256:495 (1975) or can, e.g., be isolated from phage libraries using the techniques as described herein, for example. Other methods for the preparation of clonal cell lines and of monoclonal antibodies expressed thereby are well known in the art (see, for example, Chapter 11 in: Short Protocols in Molecular Biology, (2002) 5th Ed., Ausubel F M et al., supra).

Methods for producing and screening for specific antibodies using hybridoma technology are routine and well known in the art. For example, in the hybridoma method, a mouse or other appropriate host animal, such as a sheep, goat, rabbit, rat, hamster or macaque monkey, is immunized to elicit lymphocytes that produce or are capable of producing antibodies that will specifically bind to the protein (e.g., GITR (e.g., human GITR)) used for immunization. Alternatively, lymphocytes may be immunized in vitro. Lymphocytes then are fused with myeloma cells using a suitable fusing agent, such as polyethylene glycol, to form a hybridoma cell (Goding, J W (Ed.), Monoclonal Antibodies: Principles and Practice, pp. 59-103 (Academic Press, 1986)). Additionally, a RIMMS (repetitive immunization multiple sites) technique can be used to immunize an animal (Kilpatrick, K E et al., Hybridoma 16:381-9 (1997), incorporated by reference in its entirety).

In some embodiments, mice (or other animals, such as rats, monkeys, donkeys, pigs, sheep, hamster, or dogs) can be immunized with an antigen (e.g., GITR (e.g., human GITR)) and once an immune response is detected, e.g., antibodies specific for the antigen are detected in the mouse serum, the mouse spleen is harvested and splenocytes isolated. The splenocytes are then fused by well-known techniques to any suitable myeloma cells, for example cells from cell line SP20 available from the American Type Culture Collection (ATCC) (Manassas, Va.), to form hybridomas. Hybridomas are selected and cloned by limited dilution. In certain embodiments, lymph nodes of the immunized mice are harvested and fused with NS0 myeloma cells.

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Specific embodiments employ myeloma cells that fuse efficiently, support stable high-level production of antibody by the selected antibody-producing cells, and are sensitive to a medium such as HAT medium. Among these myeloma cell lines are murine myeloma lines, such as NS0 cell line or those derived from MOPC-21 and MPC-11 mouse tumors available from the Salk Institute Cell Distribution Center, San Diego, Calif., USA, and SP-2 or X63-Ag8.653 cells available from the ATCC. Human myelotna and mouse-human heteromyeloma cell lines also have been described for the production of human monoclonal antibodies (Kozbor, D, J Immunol 133: 3001-5 (1984); Brodeur, et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against GITR (e.g., human GITR). The binding specificity of monoclonal antibodies produced by hybridoma cells is determined by methods known in the art, for example, immunoprecipitation or by an in vitro binding assay, such as radioimmunoassay (RIA) or enzyme-linked immunoabsorbent assay (ELISA).

After hybridoma cells are identified that produce antibodies of the desired specificity, affinity, and/or activity, the clones may be subcloned by limiting dilution procedures and grown by standard methods (Goding, J W (Ed,), Monoclonal Antibodies: Principles and Practice, supra). Suitable culture media for this purpose include, for example, D-MEM or RPMI 1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Antibodies described herein can be generated by any technique known to those of skill in the art. For example, Fab and F(ab′)₂ fragments described herein can be produced by proteolytic cleavage of immunoglobulin molecules, using enzymes such as papain (to produce Fab fragments) or pepsin (to produce F(ab′)₂ fragments). A Fab fragment corresponds to one of the two identical arms of a tetrameric antibody molecule and contains the complete light chain paired with the VH and CH1 domains of the heavy chain. A F(ab′)₂ fragment contains the two antigen-binding arms of a tetrarneric antibody molecule linked by disulfide bonds in the hinge region.

Further, the antibodies described herein can also be generated using various phage display methods known in the art. In phage display methods, proteins are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with a scFv linker by PCR and cloned into a phagetnid vector. The vector is electroporated in E. coli and the E. coli is infected with helper phage. Phage used in these methods are typically filamentous phage including fd and M13, and the VH and VL domains are usually recombinantly fused to either the phage gene III or gene VIII Phage expressing an antibody that binds to a particular antigen can be selected or identified with antigen, e.g., using labeled antigen or antigen bound or captured to a solid surface or bead. Examples of phage display methods that can be used to make the antibodies described herein include those disclosed in Brinkman, U et al. J Immunol Methods 182:41-50 (1995); Ames, R S et al., J Immunol Methods 184:177-186 (1995); Kettleborough, C A et al., Eur J Immunol 24:952-958 (1994); Persic, L et al., Gene 187: 9-18 (1997); Burton, D R & Barbas, C F, Advan Immunol 57:191-280 (1994); PCT Application No. PCT/GB91/001134; International Publication Nos. WO 90/02809, WO 91/10737, WO 92/01047, WO 92/18619, WO 93/1 1236, WO 95/15982, WO95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743, and 5,969,108.

As described in the above references, after phage selection, the antibody coding regions from the phage can be isolated and used to generate antibodies, including human antibodies, and expressed in any desired host, including mammalian cells, insect cells, plant cells, yeast, and bacteria, e.g., as described below. Techniques to recombinantly produce antibodies such as Fab, Fab′ and F(ab′)₂ fragments can also be employed using methods known in the art such as those disclosed in PCT publication No. WO 92/22324; Mullinax R L et al., BioTechniques 12(6): 864-9 (1992); Sawai, H et al., Am Reprod Immunol 34: 26-34 (1995); and Better, M et al., Science 240: 1041-1043 (1988).

In one aspect, to generate antibodies, PCR primers including VH or VL nucleotide sequences, a restriction site, and a flanking sequence to protect the restriction site can be used to amplify the VH or VL sequences from a template, e.g., scFv clones. Utilizing cloning techniques known to those of skill in the art, the PCR amplified. VH domains can be cloned into vectors expressing a VH constant region, and the PCR amplified VL domains can be cloned into vectors expressing a VL constant region, e.g., human kappa or lambda constant regions. The VH and VL domains can also be cloned into one vector expressing the necessary constant regions. The heavy chain conversion vectors and light chain conversion vectors are then co-transfected into cell lines to generate stable or transient cell lines that express antibodies, e.g., IgG, using techniques known to those of skill in the art.

A chimeric antibody is a molecule in which different portions of the antibody are derived from different immunoglobulin molecules. For example, a chimeric antibody can contain a variable region of a mouse or rat monoclonal antibody fused to a constant region of a human antibody. Methods for producing chimeric antibodies are known in the art. See, e.g., Morrison, S L, Science 229:1202-1207 (1985); Oi, V T & Morrison, S L, BioTechniques 4:214-221 (1986); Gillies, S D et al., J Immunol Methods 125:191-202 (1989); and U.S. Pat. Nos. 5,807,715, 4,816,567, 4,816,397, and 6,331,415.

A humanized antibody is capable of binding to a predetermined antigen and which comprises a framework region having substantially the amino acid sequence of a human immunoglobulin and CDRs having substantially the amino acid sequence of a non-human immunoglobulin (e.g., a murine immunoglobulin). In particular embodiments, a humanized antibody also comprises at least a portion of an immunoglobulin constant region (Fc), typically that of a human imtnunoglobulin. The antibody also can include the CH1, hinge, CH2, CH3, and CH4 regions of the heavy chain. A humanized antibody can be selected from any class of immunoglobulins, including IgM, IgG, IgD, IgA and IgE, and any isotype, including IgG₁, IgG₂, IgG₃ and IgG₄. Humanized antibodies can be produced using a variety of techniques known in the art, including but not limited to, CDR-grafting (European Patent No. EP 239400; International Publication No. WO 91/09967; and U.S. Pat. Nos. 5,225,539, 5,530,101, and 5,585,089), veneering or resurfacing (European Patent Nos. EP 592106 and EP 519596; Padlan, E A, Mol Immunol 28(4/5):489-498 (1991); Studnicka, G M et al., Prot Engineering 7(6): 805-814 (1994); and Roguska, M A et al., PNAS 91:969-973 (1994)), chain shuffling (U.S. Pat. No. 5,565,332), and techniques disclosed in, e.g., U.S. Pat. No. 6,407,213, U.S. Pat. No. 5,766,886, International Publication No. WO 93/17105; Tan, P et al., J Immunol 169: 1119-25 (2002); Caldas, C et al., (2000) Protein Eng. 13(5): 353-60; Morea, V et al., (2000) Methods 20(3): 267-79; Baca, M et al., (1997) J Biol Chem 272(16): 10678-84; Roguska, M A et al., (1996) Protein Eng 9(10): 895 904; Couto, J R et al., (1995) Cancer Res. 55 (23 Supp): 5973s-5977s; Couto, J R et al., (1995) Cancer Res 55(8): 1717-22; Sandhu, J S (1994) Gene 150(2): 409-10 and Pedersen, J T et al., (1994) J Mol Biol 235(3): 959-73, See also U.S. Application Publication No. US 2005/0042664 A1 (Feb. 24, 2005), which is incorporated by reference herein in its entirety.

Single domain antibodies, for example, antibodies lacking the light chains, can be produced by methods well known in the art. See Riechmann, L & Muyldermans, S J Immunol 231: 25-38 (1999); Nuttall, S D et al., Curr Pharm Biotechnol 1(3): 253-263 (2000); Muyldermans, S, J Biotechnol 74(4): 277-302 (2001); U.S. Pat. No. 6,005,079; and International Publication Nos. WO 94/04678, WO 94/25591 and WO 01/44301.

Further, antibodies that immunospecifically bind to a GITR antigen can, in turn, be utilized to generate anti-idiotype antibodies that “mimic” an antigen using techniques well known to those skilled in the art. (See, e.g., Greenspan, N S & Bona, C A FASEB J 7(5): 437-444 (1989); and Nissinoff, A, Immunol 147:2429-2438 (1991)).

In particular embodiments, an antibody described herein, which binds to the same epitope of GITR (e.g., human GITR) as an anti-GITR antibody described herein, is a human antibody. In particular embodiments, an antibody described herein, which competitively blocks (e.g., in a dose-dependent manner) any one of the antibodies described herein, (e.g., pab1876 or pab1967) from binding to GITR (e.g., human GITR), is a human antibody. Human antibodies can be produced using any method known in the art. For example, transgenic mice which are incapable of expressing functional endogenous immunoglobulins, but which can express human immunoglobulin genes, can be used. In particular, the human heavy and light chain immunoglobulin gene complexes can be introduced randomly or by homologous recombination into mouse embryonic stem cells. Alternatively, the human variable region, constant region, and diversity region can be introduced into mouse embryonic stem cells in addition to the human heavy and light chain genes. The mouse heavy and light chain immunoglobulin genes can be rendered non-functional separately or simultaneously with the introduction of human immunoglobulin loci by homologous recombination. In particular, homozygous deletion of the J_{H} region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of an antigen (e.g., GITR). Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg, N & Huszar, D, Int Rev Immunol 13:65-93 (199:5). For a detailed discussion of this technology for producing human antibodies and human monoclonal antibodies and protocols for producing such antibodies, see, e.g., International Publication Nos. WO 98/24893, WO 96/34096 and WO 96/33735; and U.S. Pat. Nos. 5,413,923, 5,625,126, 5,633,425, 5,569,825, 5,661,016, 5,545,806, 5,814,318 and 5,939,598. Examples of mice capable of producing human antibodies include the Xenomouse™ (Abgenix, Inc.; U.S. Pat. Nos. 6,075,181 and 6,150,184), the HuAb-Mouse™ (Mederex, Inc./Gen Pharm; U.S. Pat. Nos. 5,545,806 and 5,569, 825), the Trans Chromo Mouse™ (Kirin) and the KM Mouse™ (Medarex/Kirin).

Human antibodies which specifically bind to GITR (e.g., human GITR) can be made by a variety of methods known in the art including phage display methods described above using antibody libraries derived from human immunoglobulin sequences. See also U.S. Pat. Nos. 4,444,887, 4,716,111, and 5,885,793; and International Publication Nos. WO 98/46645, WO 98/50433, WO98/24893, WO98/16654, WO96/34096, WO96/33735, and WO91/10741.

In some embodiments, human antibodies can be produced using mouse-human hybridomas. For example, human peripheral blood lymphocytes transformed with Epstein-Barr virus (EBV) can be fused with mouse tnyeloma cells to produce mousehuman hybridomas secreting human monoclonal antibodies, and these mousehuman hybridomas can be screened to determine ones which secrete human monoclonal antibodies that immunospecifically bind to a target antigen (e.g., GITR (e.g., human GITR)). Such methods are known and are described in the art, see, e.g., Shinmoto, H el al., Cylotechnology 46:19-23 (2004); Naganawa, Y et al., Human Antibodies 14:27-31 (2005).

### 7.3.1 Polynueleotides

In certain aspects, provided herein are polynucleotides comprising a nucleotide sequence encoding an antibody described herein or a fragment thereof (e.g., a variable light chain region and/or variable heavy chain region) that immunospecifically binds to an GITR (e.g., human GITR) antigen, and vectors, e.g., vectors comprising such polynucleotides for recombinant expression in host cells (e.g., E. coli and mammalian cells). Provided herein are polynucleotides comprising nucleotide sequences encoding any of the antibodies provided herein, as well as vectors comprising such polynucleotide sequences, e.g., expression vectors for their efficient expression in host cells, e.g., mammalian cells.

As used herein, an “isolated” polynucleotide or nucleic acid molecule is one which is separated from other nucleic acid molecules which are present in the natural source (e.g., in a mouse or a human) of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. For example, the language “substantially free” includes preparations of polynucleotide or nucleic acid molecule having less than about 15%, 10%, 5%, 2%, 1%, 0.5%, or 0.1% (in particular less than about 10%) of other material, e.g., cellular material, culture medium, other nucleic acid molecules, chemical precursors and/or other chemicals. In a specific embodiment, a nucleic acid moleculle(s) encoding an antibody described herein is isolated or purified.

In particular aspects, provided herein are polynucleotides comprising nucleotide sequences encoding antibodies, which immunospecifically bind to an GITR polypeptide (e.g., human GITR) and comprises an amino acid sequence as described herein, as well as antibodies that compete with such antibodies for binding to an GITR polypeptide (e.g., in a dose-dependent manner), or which binds to the same epitope as that of such antibodies.

In certain aspects, provided herein are polynucleotides comprising a nucleotide sequence encoding the light chain or heavy chain of an antibody described herein. The polynucleotides can comprise nucleotide sequences encoding a light chain comprising the VL FRs and CDRs of antibodies described herein. The polynucleotides can comprise nucleotide sequences encoding a heavy chain comprising the VH FRs and CDRs of antibodies described herein. In specific embodiments, a polynucleotide described herein encodes a VL domain comprising the amino acid sequence set forth in SEQ ID NO:19. In specific embodiments, a polynucleotide described herein encodes a VH domain comprising the amino acid sequence set forth in SEQ ID NO:18.

In particular embodiments, provided herein are polynucleotides comprising a nucleotide sequence encoding an anti-GITR antibody comprising three VL chain CDRs, e.g., containing VL CDR1, VL CDR2, and VL CDR3 of any one of antibodies described herein (e.g., see Table 2). In specific embodiments, provided herein are polynucleotides comprising three VH chain CDRs, e.g., containing VH CDR1, VH CDR2, and VH CDR3 of any one of antibodies described herein (e.g., see Table 1). In specific embodiments, provided herein are polynucleotides comprising a nucleotide sequence encoding an anti-GITR antibody comprising three VH chain CDRs, e.g., containing VL CDR1, VL CDR2, and VL CDR3 of any one of antibodies described herein (e.g., see Table 2) and three VH chain CDRs, e.g., containing VH CDR1, VH CDR2, and VH CDR3 of any one of antibodies described herein (e.g., see Table 1).

In particular embodiments, provided herein are polynucleotides comprising a nucleotide sequence encoding an anti-GITR antibody or a fragment thereof comprising a VL domain, e.g., containing FR1-CDR1-FR2-CDR2-FR3-CDR3-FR4, comprising an amino acid sequence described herein. In specific embodiments, provided herein are polynucleotides comprising a nucleotide sequence encoding an anti-GITR antibody or a fragment thereof comprising a VH domain, e.g., containing FR1-CDR1-FR2-CDR2-FR3-CDR3-FR4, comprising an amino acid sequence described herein.

In certain embodiments, a polynucleotide described herein comprises a nucleotide sequence encoding an antibody provided herein comprising a light chain variable region comprising an amino acid sequence described herein (e.g., SEQ ID NO:19, 21, 23, or 26), wherein the antibody immunospecifically binds to GITR (e.g., human GITR). In a certain embodiment, a polynucleotide described herein comprises a nucleotide sequence encoding antibody pab1876w, pab1967w, pab1975w, or pab1979w provided herein or a fragment thereof comprising a light chain variable region comprising an amino acid sequence described herein (e.g., SEQ ID NO:19, 21, 23, or 26).

In certain embodiments, a polynucleotide described herein comprises a nucleotide sequence encoding an antibody provided herein comprising a heavy chain variable region comprising an amino acid sequence described herein (e.g., SEQ ID NO:18, 20, 22, 24, or 25), wherein the antibody immunospecifically binds to GITR (e.g., human GITR). In a certain embodiment, a polynucleotide described herein comprises a nucleotide sequence encoding antibody pab1876w, pab1967w, pab1975w, or pab1979w provided herein or a fragment thereof comprising a heavy chain variable region comprising an amino acid sequence described herein (e.g., SEQ ID NO: 18, 20, 22, 24, or 25).

In certain aspects, a polynucleotide comprises a nucleotide sequence encoding an antibody or fragment thereof described herein comprising a VL domain comprising one or more VL FRs having the amino acid sequence described herein, wherein the antibody immunospecifically binds to GITR. (e.g., human GITR.). In certain aspects, a polynucleotide comprises a nucleotide sequence encoding an antibody or fragment thereof described herein comprising a VH domain comprising one or more VH FRs having the amino acid sequence described herein, wherein the antibody immunospecifically binds to GITR (e.g., human GITR).

In specific embodiments, a polynucleotide provided herein comprises a nucleotide sequence encoding an antibody or fragment thereof described herein comprising: framework regions (e.g., framework regions of the VL domain and VH domain) that are human framework regions, wherein the antibody immunospecifically binds GITR (e.g., human GITR). In certain embodiments, a polynucleotide provided herein comprises a nucleotide sequence encoding an antibody or fragment thereof (e.g., CDRs or variable domain) described in Section 7.2 above.

In specific aspects, provided herein is a polynucleotide comprising a nucleotide sequence encoding an antibody comprising a light chain and a heavy chain, e.g., a separate light chain and heavy chain. With respect to the light chain, in a specific embodiment, a polynucleotide provided herein comprises a nucleotide sequence encoding a kappa light chain. In another specific embodiment, a polynucleotide provided herein comprises a nucleotide sequence encoding a lambda light chain. In yet another specific embodiment, a polynucleotide provided herein comprises a nucleotide sequence encoding an antibody described herein comprising a human kappa light chain or a human lambda light chain. In a particular embodiment, a polynucleotide provided herein comprises a nucleotide sequence encoding an antibody, which immunospecifically binds to GITR (e.g., human GITR), wherein the antibody comprises a light chain, wherein the amino acid sequence of the VL domain can comprise the amino acid sequence set forth in SEQ ID NO: 19, 21, 23, or 26 and wherein the constant region of the light chain comprises the amino acid sequence of a human kappa light chain constant region. In another particular embodiment, a polynucleotide provided herein comprises a nucleotide sequence encoding an antibody, which immunospecifically binds to GITR (e.g., human GITR), and comprises a light chain, wherein the amino acid sequence of the VL domain can comprise the amino acid sequence set forth in SEQ ID NO: 19, 21, 23, or 26, and wherein the constant region of the light chain comprises the amino acid sequence of a human lambda light chain constant region. For example, human constant region sequences can be those described in U.S. Pat. No. 5,693,780.

In a particular embodiment, a polynucleotide provided herein comprises a nucleotide sequence encoding an antibody described herein, which immunospecifically binds to GITR (e.g., human GITR), wherein the antibody comprises a heavy chain, wherein the amino acid sequence of the VH domain can comprise the amino acid sequence set forth in SEQ ID NO: 18, 20, 22, 24, or 25, and wherein the constant region of the heavy chain comprises the amino acid sequence of a human gamma (γ) heavy chain constant region.

In a certain embodiment, a polynucleotide provided herein comprises a nucleotide sequence(s) encoding a VH domain and/or a VL domain of an antibody described herein (e.g., pab1876w, pab1967w, pab1975w, or pab1979w such as SEQ ID NO: 18, 20, 22, 24, or 25 for the VH domain or SEQ ID NO: 19, 21, 23, or 26 for the VL domain), which immunospecifically binds to GITR (e.g., human GITR).

In yet another specific embodiment, a polynucleotide provided herein comprises a nucleotide sequence encoding an antibody described herein, which immunospecifically binds GITR (e.g., human GITR), wherein the antibody comprises a VL domain and a VH domain comprising any amino acid sequences described herein, and wherein the constant regions comprise the amino acid sequences of the constant regions of a human IgG₁ (e.g., allotype 1, 17, or 3), human IgG₂, or human IgG₄.

In a specific embodiment, provided herein are polynucleotides comprising a nucleotide sequence encoding an anti-GITR antibody or domain thereof, designated herein, see, e.g., Tables 1-5, for example antibody pab1876w, pab1967w, pab1975w, or pab1979w.

Also provided herein are polynucleotides encoding an anti-GITR antibody or a fragment thereof that are optimized, e.g., by codon/RNA optimization, replacement with heterologous signal sequences, and elimination of mRNA instability elements. Methods to generate optimized nucleic acids encoding an anti-GITR antibody or a fragment thereof (e.g., light chain, heavy chain, VH domain, or VL domain) for recombinant expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In some embodiments, it can be desirable to alter one or more codons to encode a conservative mutation, e.g., a similar amino acid with similar chemical structure and properties and/or function as the original amino acid.

In certain embodiments, an optimized polynucleotide sequence encoding an anti-GITR antibody described herein or a fragment thereof (e.g., VL domain or VH domain) can hybridize to an antisense (e.g., complementary) polynucleotide of an unoptimized polynucleotide sequence encoding an anti-GITR antibody described herein or a fragment thereof (e.g., VL domain or VH domain). In specific embodiments, an optimized nucleotide sequence encoding an anti-GITR antibody described herein or a fragment hybridizes under high stringency conditions to antisense polynucleotide of an unoptimized polynucleotide sequence encoding an anti-GITR antibody described herein or a fragment thereof. In a specific embodiment, an optimized nucleotide sequence encoding an anti-GITR antibody described herein or a fragment thereof hybridizes under high stringency, intermediate or lower stringency hybridization conditions to an anti sense polynucleotide of an unoptimized nucleotide sequence encoding an anti-GITR antibody described herein or a fragment thereof. Information regarding hybridization conditions has been described, see, e.g., U.S. Patent Application Publication No. US 2005/0048549 (e,g, paragraphs 72-73), which is incorporated herein by reference.

The polynucleotides can be obtained, and the nucleotide sequence of the polynucleotides determined, by any method known in the art. Nucleotide sequences encoding antibodies described herein, e.g., antibodies described in Tables 1-5, and modified versions of these antibodies can be determined using methods well known in the art, i.e., nucleotide codons known to encode particular amino acids are assembled in such a way to generate a nucleic acid that encodes the antibody. Such a polynucleotide encoding the antibody can be assembled from chemically synthesized oligonucleotides (e.g., as described in Kutmeier, G et at., BioTechniques 17:242-246 (1994)), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody or fragment thereof described. herein can be generated from nucleic acid from a suitable source (e.g., a hybridoma) using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the antibody of interest. Such PCR amplification methods can be used to obtain nucleic acids comprising the sequence encoding the light chain and/or heavy chain of an antibody. Such PCR amplification methods can be used to obtain nucleic acids comprising the sequence encoding the variable light chain region and/or the variable heavy chain region of an antibody. The amplified nucleic acids can be cloned into vectors for expression in host cells and for further cloning, for example, to generate chimeric and humanized antibodies.

If a clone containing a nucleic acid encoding a particular antibody or fragment thereof is not available, but the sequence of the antibody molecule or fragment thereof is known, a nucleic acid encoding the immunoglobulin or fragment can be chemically synthesized or obtained from a suitable source (e.g., an antibody cDNA library or a cDNA library generated from, or nucleic acid, preferably poly A+ RNA, isolated from, any tissue or cells expressing the antibody, such as hybridoma cells selected to express an antibody described herein) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR can then be cloned into replicable cloning vectors using any method well known in the art.

DNA encoding anti-GITR antibodies described herein can be readily isolated and sequenced using conventional procedures (e.g., by using oligonucleotide probes that are capable of binding specifically to genes encoding the heavy and light chains of the anti-GITR antibodies). Hybridoma cells can serve as a source of such DNA. Once isolated, the DNA can be placed into expression vectors, which are then transfected into host cells such as E. coli cells, simian COS cells, Chinese hamster ovary (CHO) cells (e.g., CHO cells from the CHO GS System™ (Lonza)), or myeloma cells that do not otherwise produce immunoglobulin protein, to obtain the synthesis of anti-GITR antibodies in the recombinant host cells.

To generate antibodies, PCR primers including VH or VL nucleotide sequences, a restriction site, and a flanking sequence to protect the restriction site can be used to amplify the VH or VL sequences in say clones. Utilizing cloning techniques known to those of skill in the art, the PCR amplified VH domains can be cloned into vectors expressing a heavy chain constant region, e.g., the human gamma 4 constant region, and the PCR amplified VL domains can be cloned into vectors expressing a light chain constant region, e.g., human kappa or lambda constant regions. In certain embodiments, the vectors for expressing the VH or VL domains comprise an EF-1α promoter, a secretion signal, a cloning site for the variable domain, constant domains, and a selection marker such as neomycin. The VH and VL, domains can also be cloned into one vector expressing the necessary constant regions. The heavy chain conversion vectors and light chain conversion vectors are then co-transfected into cell lines to generate stable or transient cell lines that express kill-length antibodies, e.g., IgG, using techniques known to those of skill in the art.

The DNA also can be modified, for example, by substituting the coding sequence for human heavy and light chain constant domains in place of the murine sequences, or by covalently joining to the immunoglobulin coding sequence all or part of the coding sequence for a non-immunoglobulin polypeptide.

Also provided are polynucleotides that hybridize under high stringency, intermediate or lower stringency hybridization conditions to polynucleotides that encode an antibody described herein. In specific embodiments, polynucleotides described herein hybridize under high stringency, intermediate or lower stringency hybridization conditions to polynucleotides encoding a VH domain (e.g., SEQ ID NO: 18, 20, 22, 24, or 25) and/or VL domain (e.g., SEQ ID NO: 19, 21, 23, or 26) provided herein.

Hybridization conditions have been described in the art and are known to one of skill in the art. For example, hybridization under stringent conditions can involve hybridization to filter-bound DNA in 6× sodium chloride/sodium citrate (SSC) at about 45° C. followed by one or more washes in 0.2×SSC/0.1% SDS at about 50-65° C.; hybridization under highly stringent conditions can involve hybridization to filter-bound nucleic acid in 6×SSC at about 45° C. followed by one or more washes in 0.1×SSC/0.2% SDS at about 68° C. Hybridization under other stringent hybridization conditions are known to those of skill in the art and have been described, see, for example, Ausubel, F M et al., eds., (1989) Current Protocols in Molecular Biology, Vol. I, Green Publishing Associates, Inc. and John Wiley & Sons, Inc., New York at pages 6.3.1-6.3.6 and 2.10.3.

### 7.3.2 Cells and Vectors

In certain aspects, provided herein are cells (e.g., host cells) expressing (e.g., recombinantly) antibodies described herein, which specifically bind to GITR (e.g., human GITR) and related polynucleotides and expression vectors. Provided herein are vectors (e.g., expression vectors) comprising polynucleotides comprising nucleotide sequences encoding anti-GITR antibodies or a fragment for recombinant expression in host cells, preferably in mammalian cells. Also provided herein are host cells comprising such vectors for recombinantly expressing anti-GITR antibodies described herein (e.g., human or humanized antibody). In a particular aspect, provided herein are methods for producing an antibody described herein, comprising expressing such antibody in a host cell.

Recombinant expression of an antibody or fragment thereof described herein (e.g., a heavy or light chain of an antibody described herein) that specifically binds to GITR (e.g., human GITR) involves construction of an expression vector containing a polynucleotide that encodes the antibody or fragment. Once a polynucleotide encoding an antibody or fragment thereof (e.g., heavy or light chain variable domains) described herein has been obtained, the vector for the production of the antibody molecule can be produced by recombinant DNA technology using techniques well known in the art. Thus, methods for preparing a protein by expressing a polynucleotide containing an antibody or antibody fragment (e.g., light chain or heavy chain) encoding nucleotide sequence are described herein. Methods which are well known to those skilled in the art can be used to construct expression vectors containing antibody or antibody fragment (e.g., light chain or heavy chain) coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding an antibody molecule described herein, a heavy or light chain of an antibody, a heavy or light chain variable domain of an antibody or a fragment thereof, or a heavy or light chain CDR, operably linked to a promoter. Such vectors can, for example, include the nucleotide sequence encoding the constant region of the antibody molecule (see, e.g., international Publication Nos. WO 86/05807 and WO 89/01036; and U.S. Pat. No. 5,122,464) and variable domains of the antibody can be cloned into such a vector for expression of the entire heavy, the entire light chain, or both the entire heavy and light chains.

An expression vector can be transferred to a cell (e.g., host cell) by conventional techniques and the resulting cells can then be cultured by conventional techniques to produce an antibody described herein (e.g., an antibody comprising the CDRs of pab1876w, pab1967w, pab1975w, or pab1979w) or a fragment thereof. Thus, provided herein are host cells containing a polynucleotide encoding an antibody described herein (e.g., an antibody comprising the CDRs of pab1876w, pab1967w, pab1975w, or pab1979w) or fragments thereof (e.g., a heavy or light chain thereof, or fragment thereof), operably linked to a promoter for expression of such sequences in the host cell. In certain embodiments, for the expression of double-chained antibodies, vectors encoding both the heavy and light chains, individually, can be co-expressed in the host cell for expression of the entire immunoglobulin molecule, as detailed below. In certain embodiments, a host cell contains a vector comprising a polynucleotide encoding both the heavy chain and light chain of an antibody described herein (e.g., an antibody comprising the CDRs of pab1876w, pab1967w, pab1975w, or pab1979w), or a fragment thereof. In specific embodiments, a host cell contains two different vectors, a first vector comprising a polynucleotide encoding a heavy chain or a heavy chain variable region of an antibody described herein (e.g., an antibody comprising the CDRs of pab1876w, pab1967w, pab1975w, or pab1979w), or a fragment thereof, and a second vector comprising a polynucleotide encoding a light chain or a light chain variable region of an antibody described herein (e.g., an antibody comprising the CDRs of pab1876w, pab1967w, pab1975w, or pab1979w), or a fragment thereof. In other embodiments, a first host cell comprises a first vector comprising a polynucleotide encoding a heavy chain or a heavy chain variable region of an antibody described herein (e.g., an antibody comprising the CDRs of pab1876w, pab1967w, pab1975w, or pab1979w), or a fragment thereof, and a second host cell comprises a second vector comprising a polynucleotide encoding a light chain or a light chain variable region of an antibody described herein (e.g., an antibody comprising the CDRs of pab1876w, pab1967w pab1975w, or pab1979w). In specific embodiments, a heavy chain/heavy chain variable region expressed by a first cell associated with a light chain/light chain variable region of a second cell to form an anti-GITR antibody described herein (e.g., antibody comprising the CDRs pab1876w, pab1967w, pab1975w, or pab1979w). In certain embodiments, provided herein is a population of host cells comprising such first host cell and such second host cell.

In a particular embodiment, provided herein is a population of vectors comprising a first vector comprising a polynucleotide encoding a light chain/light chain variable region of an anti-GITR antibody described herein (e.g., antibody comprising the CDRs of pab1876w, pab1967w, pab1975w, or pab1979w), and a second vector comprising a polynucleotide encoding a heavy chain/heavy chain variable region of an anti-GITR antibody described herein (e.g., antibody comprising the CDRs of pab 1876w, pab1967w, pab1975w, or pab1979w).

A variety of host-expression vector systems can be utilized to express antibody molecules described herein (e.g., an antibody comprising the CDRs of pab1876w, pab1967w, pab1975w, or pab1979w) (see, e.g., U.S. Pat. No. 5,807,715). Such host-expression systems represent vehicles by which the coding sequences of interest can be produced and subsequently purified, but also represent cells which can, when transformed or transfected with the appropriate nucleotide coding sequences, express an antibody molecule described herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors e.g., baculovirus) containing antibody coding sequences; plant cell systems (e.g., green algae such as Chlamydomonas reinhardth) infected with recombinant virus expression vectors (e.g., cauliflower mosaic virus, CaMV, tobacco mosaic virus, TMV) or transformed with recombinant plasmid expression vectors (e.g., Ti plasmid) containing antibody coding sequences; or mammalian cell systems (e.g., COS (e.g., COS1 or COS), CHO, BHK, MDCK, HEK 293, NS0, PER.C6, VERO, CRL7O3O, HsS78Bst, HeLa, and NIH 3T3, HepG2, SP210, R1.1, B-W, L-M, BSC1, BSC40, YB/20 and BMT10 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In a specific embodiment, cells for expressing antibodies described herein (e.g., an antibody comprising the CDRs of any one of antibodies pab1876w, pab1967w, pab1975w, or pab1979w) are CHO cells, for example CHO cells from the CHO GS System™ (Lonza). In a particular embodiment, cells for expressing antibodies described herein are human cells, e.g., human cell lines. In a specific embodiment, a mammalian expression vector is pOptiVEC™ or pcDNA3.3. In a particular embodiment, bacterial cells such as Escherichia coli, or eukaryotic cells (e.g., mammalian cells), especially for the expression of whole recombinant antibody molecule, are used for the expression of a recombinant antibody molecule. For example, mammalian cells such as Chinese hamster ovary (CHO) cells in conjunction with a vector such as the major intermediate early gene promoter element from human cytomegalovirus is an effective expression system for antibodies (Foecking, M K & Hofstetter, H Gene 45: 101-105 (1986); and Cockett, M I et al., Biotechnology 8: 662-667 (1990)). In certain embodiments, antibodies described herein are produced by CHO cells or NS0 cells. In a specific embodiment, the expression of nucleotide sequences encoding antibodies described herein which immunospecifically bind GITR (e.g., human GITR) is regulated by a constitutive promoter, inducible promoter or tissue specific promoter.

In bacterial systems, a number of expression vectors can be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified can be desirable. Such vectors include, but are not limited to, the E. coli expression vector pUR278 (Ruether, U & Mueller-Hill, B, EMBO J 2:1791-1794 (1983)), in which the antibody coding sequence can be ligated individually into the vector in frame with the lac Z coding region so that a fusion protein is produced; pIN vectors (Inouye, S & Inouye, M. Nuc Acids Res 13: 3101-3109 (1985); Van. Heeke G & Schuster, S M, J Biol Chem 24: 5503-5509 (1989)); and the like. For example, pGEX vectors can also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV), for example, can be used as a vector to express foreign genes. The virus grows in Spodoptera frugiperda cells. The antibody coding sequence can be cloned individually into non-essential regions (for example the polyhedrin gene) of the virus and placed under control of an AcNPV promoter (for example the polyhedrin promoter).

In mammalian host cells, a number of viral-based expression systems can be utilized. In cases where an adenovirus is used as an expression vector, the antibody coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan, J & Shenk, T, PNAS 81:3655-3659 (1984)). Specific initiation signals can also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bitter, G et al., Methods Enzymol 153:516-544 (1987)).

In addition, a host cell strain can be chosen which modulates the expression of the inserted sequences, or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT2O and T47D, NS0 (a murine myeloma cell line that does not endogenously produce any immunoglobulin chains), CRL7O3O, COS (e.g., COS1 or COS), PER.C6, VERO, HsS78Bst, HEK-293T, HepG2, SP210, R1.1 B-W, L-M, BSC1, BSC40, YB/20, BMT10 and HsS78Bst cells. In certain embodiments, anti-GITR antibodies described herein (e.g., an antibody comprising the CDRs of pab1876w, pab1967w, pab1975w, or pab1979w) are produced in mammalian cells, such as CHO cells.

In a specific embodiment, the antibodies described herein have reduced fucose content or no fucose content. Such antibodies can be produced using techniques known one skilled in the art. For example, the antibodies can be expressed in cells deficient or lacking the ability of to fucosylate. In a specific example, cell lines with a knockout of both alleles of α1,6-fucosyltransferase can be used to produce antibodies with reduced fucose content. The Potelligent® system (Lorna) is an example of such a system that can be used to produce antibodies with reduced fucose content.

For long-term, high-yield production of recombinant proteins, stable expression cells can be generated. For example, cell lines which stably express an anti-GITR antibody described herein (e.g., an antibody comprising the CDRs of pab1876w, pab1967w, pab1975w, or pab1979w) can be engineered. In specific embodiments, a cell provided herein stably expresses a light chain/light chain variable domain and a heavy chain/heavy chain variable domain which associate to form an antibody described herein (e.g., an antibody comprising the CDRs of pab1876w, pab1967w, pab1975w, or pab1979w).

In certain aspects, rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA/potynucleotide, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express an anti-GITR antibody described herein or a fragment thereof. Such engineered cell lines can be particularly useful in screening and evaluation of compositions that interact directly or indirectly with the antibody molecule.

A number of selection systems can be used, including but not limited to, the herpes simplex virus thymidine kinase (Wigler M et al., (1977) Cell 11(1): 223-232), hypoxanthineguanine phosphoribosyltransferase (Szybalska E H & Szybalski W (1962) PNAS 48(12): 2026-2034) and adenine phosphoribosyltransferase (Lowy I et al., (1980) Cell 22(3): 817-823) genes can be employed in tk-, hgprt- or aprt-cells, respectively. Also, antimetabolite resistance can be used as the basis of selection for the following genes: dhfr, which confers resistance to methotrexate (Wigler, M et al., (1980) PNAS 77(6): 3567-3570; O'Hare, K et al., (1981) PNAS 78: 1527-1531); gpt, which confers resistance to mycophenolic acid (Mulligan, R C & Berg, P (1981) PNAS 78(4): 2072-2076); neo, which confers resistance to the aminoglycoside G-418 (Wu, G Y & Wu, CH (1991) Biotherapy 3: 87-95; Tolstoshev, P (1993) Ann Rev Pharmacol Toxicol 32: 573-596; Mulligan, R C (1993) Science 260: 926-932; and Morgan, R A & Anderson, W F (1993) Ann Rev Biochem 62: 191-217; Nabel, G J & Feigner, P L (1993) Trends Biotechnol 11(5): 211-215); and hygro, which confers resistance to hygromycin (Santerre, R F et al., (1984) Gene 30(1-3): 147-156). Methods commonly known in the art of recombinant DNA technology can be routinely applied to select the desired recombinant clone and such methods are described, for example, in Ausubel, F M et al., (eds.), Current Protocols in Molecular Biology, John Wiley & Sons, NY (1993); Kriegler, M, Gene Transfer and Expression, A Laboratory Manual, Stockton Press, NY (1990); and in Chapters 12 and 13, Dracopoli, N C et al., (eds.), Current Protocols in Human Genetics, John Wiley & Sons, NY (1994); Colbere-Garapin, F et al. J Mol Biol 150: 1-14 (1981), which are incorporated by reference herein in their entireties.

The expression levels of an antibody molecule can be increased by vector amplification (for a review, see Bebbington, C R & Hentschel, C C G, The use of vectors based on gene amplification for the expression of cloned genes in mammalian cells in DNA cloning, Vol, 3 (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse, G F et al., Mol Cell Biol 3:257-266 (1983)).

The host cell can be co-transfected with two or more expression vectors described herein, the first vector encoding a heavy chain derived polypeptide and the second vector encoding a light chain derived polypeptide. The two vectors can contain identical selectable markers which enable equal expression of heavy and light chain polypeptides. The host cells can be co-transfected with different amounts of the two or more expression vectors. For example, host cells can be transfected with any one of the following ratios of a first expression vector and a second expression vector: 1:1, 1:2, 1:3, 1:4, 1:5, 1:6, 1:7, 1:8, 1:9, 1:10, 1:12, 1:15, 1:20, 1:30, 1:35, 1:40, 1:45, or 1:50.

Alternatively, a single vector can be used which encodes, and is capable of expressing, both heavy and light chain polypeptides. In such situations, the light chain should be placed before the heavy chain to avoid an excess of toxic free heavy chain (Proudfoot, N J, Nature 322:562-565 (1986); and Köhler, G PNAS 77: 2197-2199 (1980)). The coding sequences for the heavy and light chains can comprise cDNA or genomic DNA. The expression vector can be monocistronic or multicistronic. A multicistronic nucleic acid construct can encode 2, 3, 4, 5, 6, 7, 8, 9, 10 or more, or in the range of 2-5, 5-10 or 10-20 genes/nucleotide sequences. For example, a bicistronic nucleic acid construct can comprise in the following order a promoter, a first gene (e.g., heavy chain of an antibody described herein), and a second gene and (e.g., light chain of an antibody described herein). In such an expression vector, the transcription of both genes can be driven by the promoter, whereas the translation of the mRNA from the first gene can be by a cap-dependent scanning mechanism and the translation of the mRNA from the second gene can be by a cap-independent mechanism, e.g., by an IRES.

Once an antibody molecule described herein has been produced by recombinant expression, it can be purified by any method known in the art for purification of an immunoglobulin molecule, for example, by chromatography (e.g., ion exchange, affinity, particularly by affinity for the specific antigen after Protein A, and sizing column chromatography), centrifugation, differential solubillity, or by any other standard technique for the purification of proteins. Further, the antibodies described herein can be fused to heterologous polypeptide sequences described herein or otherwise known in the art to facilitate purification.

In specific embodiments, an antibody described herein is isolated or purified. Generally, an isolated antibody is one that is substantially free of other antibodies with different antigenic specificities than the isolated antibody. For example, in a particular embodiment, a preparation of an antibody described herein is substantially free of cellular material and/or chemical precursors. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, 5%, 2%, 1%, 0.5%, or 0.1% (by dry weight) of heterologous protein (also referred to herein as a “contaminating protein”) and/or variants of an antibody, for example, different post-translational modified forms of an antibody. When the antibody or fragment is recombinantly produced, it is also generally substantially free of culture medium, i.e., culture medium represents less than about 20%, 10%, 2%, 1%, 0.5%, or 0.1% of the volume of the protein preparation. When the antibody or fragment is produced by chemical synthesis, it is generally substantially free of chemical precursors or other chemicals, i.e., it is separated from chemical precursors or other chemicals which are involved in the synthesis of the protein. Accordingly, such preparations of the antibody or fragment have less than about 30%, 20%, 10%, or 5% (by dry weight) of chemical precursors or compounds other than the antibody or fragment of interest. In a specific embodiment, antibodies described herein are isolated or purified.

### 7.4 Pharmaceutical Compositions

Provided herein are compositions comprising an antibody described herein having the desired degree of purity in a physiologically acceptable carrier, excipient, or stabilizer (Remington's Pharmaceutical Sciences (1990) Mack Publishing Co., Easton, Pa.). Acceptable carriers, excipients, or stabilizers are nontoxic to recipients at the dosages and concentrations employed.

Pharmaceutical compositions described herein that comprise an antagonistic antibody described herein can be useful in reducing, deactivating, or inhibiting GITR activity and treating a condition such as an inflammatory or autoimmune disease or disorder or an infectious disease. Pharmaceutical compositions as described herein that comprise an antibody described herein can be useful in reducing, inhibiting, or deactivating a GITR activity and treating a condition selected from the group consisting of infections (viral, bacterial, fungal and parasitic), endotoxic shock associated with infection, arthritis, rheumatoid arthritis, asthma, chronic obstructive pulmonary disease (COPD), pelvic inflammatory disease, Alzheimer's Disease, inflammatory bowel disease, Crohn's disease, ulcerative colitis, Peyronie's Disease, coeliac disease, gallbladder disease, Pilonidal disease, peritonitis, psoriasis, vasculitis, surgical adhesions, stroke, Type I Diabetes, lyme disease, arthritis, meningoencephalitis, uveitis, autoimmune uveitis, immune mediated inflammatory disorders of the central and peripheral nervous system such as multiple sclerosis, lupus (such as systemic lupus erythematosus) and Guillain-Barr syndrome, dermatitis, Atopic dermatitis, autoimmune hepatitis, fibrosing alveolitis, Grave's disease, IgA nephropathy, idiopathic thrombocytopenic purpura, Meniere's disease, pemphigus, primary biliary cirrhosis, sarcoidosis, scleroderma, Wegener's granulomatosis, pancreatitis, trauma (surgery), graft-versus-host disease, transplant rejection, heart disease (i.e., cardiovascular disease) including ischaemic diseases such as myocardial infarction as well as atherosclerosis, intravascular coagulation, bone resorption, osteoporosis, osteoarthritis, periodontitis, hypochlorhydi a, and neuromyelitis optica.

The compositions to be used for in vivo administration can be sterile. This is readily accomplished by filtration through, e.g., sterile filtration membranes.

### 7.5 Uses and Methods

### 7.5.1 Therapeutic Uses and Methods

In one aspect, presented herein are methods for modulating one or more immune functions or responses in a subject, comprising to a subject in need thereof administering an antibody that binds to GITR described herein (e.g., an anti-GITR antagonistic antibody, e.g., an anti-GITR-monovalent antibody) or a composition comprising such an antibody.

In one aspect, the methods for modulating one or more immune functions or responses in a subject as presented herein are methods for deactivating, reducing, or inhibiting one or more immune functions or responses in a subject, comprising to a subject in need thereof administering an anti-GITR antagonistic antibody or a composition thereof as described herein. In a specific embodiment, presented herein are methods for preventing and/or treating diseases in which it is desirable to deactivate, reduce, or inhibit one or more immune functions or responses, comprising administering to a subject in need thereof an anti-GITR antagonistic antibody described herein or a composition thereof. In a certain embodiment, presented herein are methods of treating an autoimmune or inflammatory disease or disorder comprising administering to a subject in need thereof an effective amount of an anti-GITR antagonistic antibody or a composition thereof as described herein. In a certain embodiment, presented herein are methods of treating an infectious disease comprising administering to a subject in need thereof an effective amount of an anti-GITR antagonistic antibody or a composition thereof as described herein. In certain embodiments, the subject is a human. In certain embodiments, the disease or disorder is selected from the group consisting of: infections (viral, bacterial, fungal and parasitic), endotoxic shock associated with infection, arthritis, rheumatoid arthritis, asthma, chronic obstructive pulmonary disease (COPD), pelvic inflammatory disease, Alzheimer's Disease, inflammatory bowel disease, Crohn's disease, ulcerative colitis, Peyronie's Disease, coeliac disease, gallbladder disease, Pilonidal disease, peritonitis, psoriasis, vasculitis, surgical adhesions, stroke, Type I Diabetes, lyme disease, arthritis, meningoencephalitis, uveitis, autoimmune uveitis, immune mediated inflammatory disorders of the central and peripheral nervous system such as multiple sclerosis, lupus (such as systemic lupus erythematosus) and. Guillain-Barr syndrome, dermatitis, Atopic dermatitis, autoimmune hepatitis, fibrosing alveolitis, Grave's disease, IgA nephropathy, idiopathic thrombocytopenic purpura, Meniere's disease, pemphigus, primary biliary cirrhosis, sarcoidosis, scleroderma, Wegener's granulomatosis, pancreatitis, trauma (surgery), graft-versus-host disease, transplant rejection, heart disease (i.e., cardiovascular disease) including ischaemic diseases such as myocardial infarction as well as atherosclerosis, intravascular coagulation, bone resorption, osteoporosis, osteoarthritis, periodontitis, hypochlorhydia, and neuromyelitis optica. In certain embodiments, the disease or disorder is selected from the group consisting of: transplant rejection, graft-versus-host disease, vasculitis, asthma, rheumatoid arthritis, dermatitis, inflammatory bowel disease, uveitis, lupus, colitis, diabetes, multiple sclerosis, and airway inflammation.

In another embodiment, an anti-GITR antagonistic antibody is administered to a patient diagnosed with an autoimmune or inflammatory disease or disorder to decrease the proliferation and/or effector function of one or more immune cell populations (e.g., T cell effector cells, such as CD4⁺ and CD8⁺ T cells) in the patient. [002511 In a specific embodiment, an anti-GITR antagonistic antibody described herein deactivates or reduces or inhibits one or more immune functions or responses in a subject by at least 99%, at least 98%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10%, or in the range of between 10% to 25%, 25% to 50%, 50% to 75%, or 75% to 95% relative to the immune function in a subject not administered the anti-GITR antagonistic antibody described herein using assays well known in the art, e.g., ELISPOT, ELISA, and cell proliferation assays. In a specific embodiment, the immune function is cytokine production (e.g., IL-2, TNF-α, IFN-γ, IL-4, IL-10, and/or IL-13 production). In another embodiment, the immune function is T cell proliferation/expansion, which can be assayed, e.g., by flow cytometry to detect the number of cells expressing markers of T cells (e.g., CD3, CD4, or CD8). In another embodiment, the immune function is antibody production, which can be assayed, e.g., by ELISA. In some embodiments, the immune function is effector function, which can be assayed, e.g., by a cytotoxicity assay or other assays well known in the art. In another embodiment, the immune function is a Thi. response. In another embodiment, the immune function is a Th2 response. In another embodiment, the immune function is a memory response.

In specific embodiments, non-limiting examples of immune functions that can be reduced or inhibited by an anti-GITR antagonistic antibody or composition thereof as described herein are proliferation/expansion of effector lymphocytes (e.g., decrease in the number of effector T lymphocytes), and stimulation of apoptosis of effector lymphocytes (e.g., effector T lymphocytes). In particular embodiments, an immune function reduced or inhibited by an anti-GITR antagonistic antibody or composition thereof as described herein is proliferation/expansion in the number of or activation of CD4⁺ T cells (e.g., Th1 and Th2 helper T cells), CD8⁺ T cells (e.g., cytotoxic T lymphocytes, alpha/beta T cells, and gamma/delta T cells), B cells (e.g., plasma cells), memory T cells, memory B cells, tumor-resident T cells, CD122⁺ T cells, natural killer (NK) cells), macrophages, monocytes, dendritic cells, mast cells, eosinophils, basophils or polymorphonucleated leukocytes. In one embodiment, an anti-GITR antagonistic antibody or composition thereof as described herein deactivates or reduces or inhibits the proliferation/expansion or number of lymphocyte progenitors. In some embodiments, an anti-GITR antagonistic antibody or composition thereof as described herein decreases the number of CD4⁺ T cells (e.g., Th1 and Th2 helper T cells), CD8⁺ T cells (e.g., cytotoxic T lymphocytes, alpha/beta T cells, and gamma/delta T cells), B cells (e.g., plasma cells), memory T cells, memory B cells, tumor-resident T cells, CD122⁺ T cells, natural killer cells (NK cells), macrophages, monocytes, dendritic cells, mast cells, eosinophils, basophils or polymorphonucleated leukocytes by approximately at least 99%, at least 98%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10%, or in the range of between 10% to 25%, 25% to 50%, 50% to 75%, or 75% to 95% relative a negative control (e.g., number of the respective cells not treated, cultured, or contacted with an anti-GITR antagonistic antibody or composition thereof as described herein).

In certain embodiments, any of the methods herein (e.g., methods of treating an infectious disease, or methods of treating an autoimmune or inflammatory disease or disorder) comprise administration to a subject of an antibody as described herein and a checkpoint targeting agent. In certain embodiments, the checkpoint targeting agent is an antibody (e.g., an anti-PD-1 antibody, an anti-PD-L1 antibody, an anti-PD-L2 antibody, an anti-CTLA-4 antibody, an anti-TIM-3 antibody, an anti-LAG-3 antibody, an anti-CEACAM1 antibody, an anti-GITR antibody, or an anti-OX40 antibody). In certain embodiments, the checkpoint targeting agent is an antagonist or agonist antibody.

### 7.5.1.1 Routes of Administration & Dosage

An antibody or composition described herein can be delivered to a subject by a variety of routes.

The amount of an antibody or composition which will be effective in the treatment and/or prevention of a condition will depend on the nature of the disease, and can be determined by standard clinical techniques.

The precise dose to be employed in a composition will also depend on the route of administration, and the seriousness of the disease, and should be decided according to the judgment of the practitioner and each subject's circumstances. For example, effective doses may also vary depending upon means of administration, target site, physiological state of the patient (including age, body weight and health), whether the patient is human or an animal, other medications administered, or whether treatment is prophylactic or therapeutic. Usually, the patient is a human but non-human mammals including transgenic mammals can also be treated. Treatment dosages are optimally titrated to optimize safety and efficacy.

In certain embodiments, an in vitro assay is employed to help identify optimal dosage ranges. Effective doses may be extrapolated from dose response curves derived from in vitro or animal model test systems.

Generally, human antibodies have a longer half-life within the human body than antibodies from other species due to the immune response to the foreign polypeptides. Thus, lower dosages of human antibodies and less frequent administration is often possible.

### 7.5.2 Detection & Diagnostic Uses

An anti-GITR antibody described herein (see, e,g., Section 7.2) can be used to assay GITR protein levels in a biological sample using classical immunohistological methods known to those of skill in the art, including immunoassays, such as the enzyme linked immunosorbent assay (ELISA), immunoprecipitation, or Western blotting. Suitable antibody assay labels are known in the art and include enzyme labels, such as, glucose oxidase; radioisotopes, such as iodine (¹²⁵I, ¹²¹I), carbon (¹⁴C), sulfur (³⁵S), tritium (³H), indium (¹²¹In), and technetium (⁹⁹Tc); luminescent labels, such as luminol; and fluorescent labels, such as fluorescein and rhodatnine, and biotin. Such labels can be used to label an antibody described herein. Alternatively, a second antibody that recognizes an anti-GITR antibody described herein can be labeled and used in combination with an anti-GITR antibody to detect GITR protein levels.

Assaying for the expression level of GITR protein is intended to include qualitatively or quantitatively measuring or estimating the level of a GPM protein in a first biological sample either directly (e.g., by determining or estimating absolute protein level) or relatively (e.g., by comparing to the disease associated protein level in a second biological sample). GITR polypeptide expression level in the first biological sample can be measured or estimated and compared to a standard GITR protein level, the standard being taken from a second biological sample obtained from an individual not having the disorder or being determined by averaging levels from a population of individuals not having the disorder. As will be appreciated in the art, once the “standard” GITR polypeptide level is known, it can be used repeatedly as a standard for comparison.

As used herein, the term “biological sample” refers to any biological sample obtained from a subject, cell line, tissue, or other source of cells potentially expressing GITR. Methods for obtaining tissue biopsies and body fluids from animals (e.g., humans) are well known in the art, Biological samples include peripheral mononuclear blood cells.

An anti-GITR antibody described herein can be used for prognostic, diagnostic, monitoring and screening applications, including in vitro and in vivo applications well known and standard to the skilled artisan and based on the present description. Prognostic, diagnostic, monitoring and screening assays and kits for in vitro assessment and evaluation of immune system status and/or immune response may be utilized to predict, diagnose and monitor to evaluate patient samples including those known to have or suspected of having an immune system-dysfunction or with regard to an anticipated or desired immune system response, antigen response or vaccine response. The assessment and evaluation of immune system status and/or immune response is also useful in determining the suitability of a patient for a clinical trial of a drug or for the administration of a particular chemotherapeutic agent or an antibody, including combinations thereof, versus a different agent or antibody. This type of prognostic and diagnostic monitoring and assessment is already in practice utilizing antibodies against the HER2 protein in breast cancer (HercepTest™, Dako) where the assay is also used to evaluate patients for antibody therapy using Herceptin®. In vivo applications include directed cell therapy and immune system modulation and radio imaging of immune responses.

In one embodiment, an anti-GITR antibody can be used in immunohistochemistry of biopsy samples.

In another embodiment, an anti-GITR antibody can be used to detect levels of GITR, or levels of cells which contain GITR on their membrane surface, which levels can then be linked to certain disease symptoms. Anti-GITR antibodies described herein may carry a detectable or functional label. When fluorescence labels are used, currently available microscopy and fluorescence-activated cell sorter analysis (FACS) or combination of both methods procedures known in the art may be utilized to identify and to quantitate the specific binding members. Anti-GITR antibodies described herein can carry a fluorescence label. Exemplary fluorescence labels include, for example, reactive and conjugated probes, e.g., Aminocoumarin, Fluorescein and Texas red, Alexa Fluor dyes, Cy dyes and DyLight dyes. An anti-GITR antibody can carry a radioactive label, such as the isotopes ³H, ¹⁴C, ³²P, ³⁵S, ³⁶Cl, ⁵¹Cr, ⁵⁷CO, ⁵⁸Co, ⁵⁹Fe, ⁶⁷Cu, ⁹⁰Y, ⁹⁹Tc, ¹¹¹In, ¹¹⁷Lu, ¹²¹I, ¹²⁴I, ¹²⁵I, ¹³¹I, ¹⁹⁸Au, ²¹¹At, ²¹³Bi, ²²⁵Ac and ¹⁸⁶Re. When radioactive labels are used, currently available counting procedures known in the art may be utilized to identify and quantitate the specific binding of anti-GITR antibody to GITR (e.g., human GITR). In the instance where the label is an enzyme, detection may be accomplished by any of the presently utilized colorimetric, spectrophotometric, fluorospectrophotometric, amperometric or gasometric techniques as known in the art. This can be achieved by contacting a sample or a control sample with an anti-GITR antibody under conditions that allow for the formation of a complex between the antibody and GITR. Any complexes formed between the antibody and GITR are detected and compared in the sample and the control. In light of the specific binding of the antibodies described herein for GITR, the antibodies thereof can be used to specifically detect GITR expression on the surface of cells. The antibodies described herein can also be used to purify GITR via immunoaffinity purification.

Also included herein is an assay system which may be prepared in the form of a test kit for the quantitative analysis of the extent of the presence of for instance, GITR or GITR/GITRL complexes. The system or test kit may comprise a labeled component, e.g., a labeled antibody, and one or more additional immunochemical reagents. See, e.g., Section 7.6 below for more on kits.

### 7.6 Kits

Provided herein are kits comprising one or more antibodies described herein or conjugates thereof. In a specific embodiment, provided herein is a pharmaceutical pack or kit comprising one or more containers filled with one or more of the ingredients of the pharmaceutical compositions described herein, such as one or more antibodies provided herein. In some embodiments, the kits contain a pharmaceutical composition described herein and any prophylactic or therapeutic agent, such as those described herein. In certain embodiments, the kits may contain a T cell mitogen, such as, e.g., phytohaemagglutinin (PHA) and/or phorbol myristate acetate (PMA), or a TCR complex stimulating antibody, such as an anti-CD3 antibody and anti-CD28 antibody. Optionally associated with such container(s) can be a notice in the form prescribed by a governmental agency regulating the manufacture, use or sale of pharmaceuticals or biological products, which notice reflects approval by the agency of manufacture, use or sale for human administration.

Also provided herein are kits that can be used in the above methods. In one embodiment, a kit comprises an antibody described herein, preferably a purified antibody, in one or more containers. In a specific embodiment, kits described herein contain a substantially isolated GITR antigen (e.g., human GITR) that can be used as a control. In another specific embodiment, the kits described herein further comprise a control antibody which does not react with a GITR antigen. In another specific embodiment, kits described herein contain one or more elements for detecting the binding of an antibody to a GITR antigen (e.g., the antibody can be conjugated to a detectable substrate such as a fluorescent compound, an enzymatic substrate, a radioactive compound or a luminescent compound, or a second antibody which recognizes the first antibody can be conjugated to a detectable substrate). In specific embodiments, a kit provided herein can include a recombinantly produced or chemically synthesized GITR antigen. The GITR antigen provided in the kit can also be attached to a solid support. In a more specific embodiment, the detecting means of the above described kit includes a solid support to which a GITR antigen is attached. Such a kit can also include a non-attached reporter-labeled anti-human antibody or anti-mouse/rat antibody. In this embodiment, binding of the antibody to the GITR. antigen can be detected by binding of the said reporter-labeled antibody.

The following examples are offered by way of illustration and not by way of limitation.

### 8. EXAMPLES

The examples in this Section (i.e., Section 8) are offered by way of illustration, and not by way of limitation.

### 8.1 Example 1: Characterization of Anti-GITR Antibody

This example describes the characterization of pab1876, an antibody that binds to human GITR, comprising a heavy chain of the amino acid sequence of SEQ ID NO: 29 and a light chain of the amino acid sequence of SEQ ID NO: 38. pab1876 is a human IgG₁ antibody containing a T109S substitution in the light chain constant domain (i.e., substitution of threonine with serine at position 109 relative to the wild type light chain constant domain), numbered according to Kabat, which facilitates the cloning of the variable region in frame to the constant region. This mutation is a conservative modification that does not affect antibody binding or function. The wild type counterpart, named pab1876w, which contains a threonine at position 109, numbered according to Kabat, was also generated. The antibody pab1876w is a human IgG₁ antibody comprising a heavy chain of SEQ ID NO: 29 and a light chain of SEQ ID NO: 37.

The activation of GITR signaling depends on receptor clustering to form higher order receptor complexes that efficiently recruit apical adapter proteins to drive intracellular signal transduction. Without being bound by theory, an anti-GITR agonist antibody may mediate receptor clustering through bivalent antibody arms and/or through Fc-Fc receptor (FcR) co-engagement on accessory myeloid or lymphoid cells. Consequently, one approach for developing an anti-GITR antagonist antibody is to select an antibody that competes with GITR ligand (GITRL) for binding to GITR, diminish or eliminate the binding of the Fc region of the antibody to Fc receptors, and/or adopt a monovalent antibody format (containing only one GITR-specific antigen-binding domain, and optionally a second antigen-binding domain that is not GITR-specific). In this example, an anti-GITR antibody pab1876w was characterized using a GITR reporter assay to first assess how much residual agonistic activity it retained in the absence of FcR interaction and second examine its ability to antagonize GITRL-induced signaling through GITR molecules. Alternatively or in addition, a monovalent antagonist antibody could be developed based on the variable region sequences of pab1876w. Monovalent antibody formats include, but are not limited to, Fab or scFv optionally fused to an Fc region or another half-life-extending moiety, e.g., poly(ethyleneglycol) (PEG) and human serum albumin (HSA).

### 8.1.1 Effect of Anti-GITR Antibody on GITR NF-κB-Luciferase Reporter Cell Line

A human GITR NF-κB-luciferase reporter cell line (Promega) was developed to test the agonistic activity of soluble pab1876w on GITR-expressing cells. This reporter assay was built using Jurkat cells which expressed minimum amount, if any, of FcR, diminishing the possibility of FeR-mediated clustering of the GITR molecules.

Jurkat cells were genetically modified to stably express the GloResponse NF-κB-luc2P construct and human GITR. Expression of GITR was verified by flow cytometry. To evaluate agonistic activity, the Jurkat-huGITR-NF-κB-luciferase reporter cells were plated at 1×10⁵ cells per well in assay media (RPMI+1% PBS) and incubated with different concentration of trimeric GITRL (2, 1.33, 0.44, 0.14, 0.049, 0.016, 0.005, 0.0018 or 0.00061 μg/ml) or a soluble antibody (12,5, 10, 5, 2.5, 1.25 or 0.625 μg/ml). The antibodies tested were the anti-GITR antibody pab1876w and an isotype control antibody. After 6-hour incubation at 37° C. and 5% CO₂, an equal volume of room temperature Bio-Glo reagent (Promega) was added. The luciferase activity was measured as relative light units (RLU) using an EnVision multilabel reader 2100.

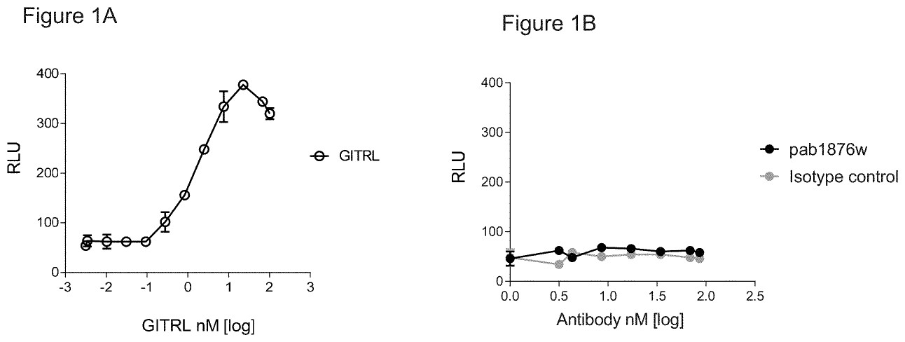

While trimeric GITRL induced NF-κB-luciferase activity over a wide range of concentrations (FIG. 1A), minimal luciferase signal was observed after incubation with soluble pab1876w (FIG. 1B).

Next, pab1876w was assessed for its ability to block NF-κB signaling induced by GITRL-expressing cells. Jurkat-huGITR-NF-κB-luciferase reporter cells were plated at 1×10⁵ cell per well in the presence or absence of 1×10⁴ FMK cells expressing GITRL and a soluble dose range of pab1876w or an isotype control antibody. After 6-hour incubation at 37° C. and 5% CO₂, an equal volume of room temperature Bio-Glo reagent (Promega) was added. Luminescence was read as RLU using an EnVision multilabel reader 2100.

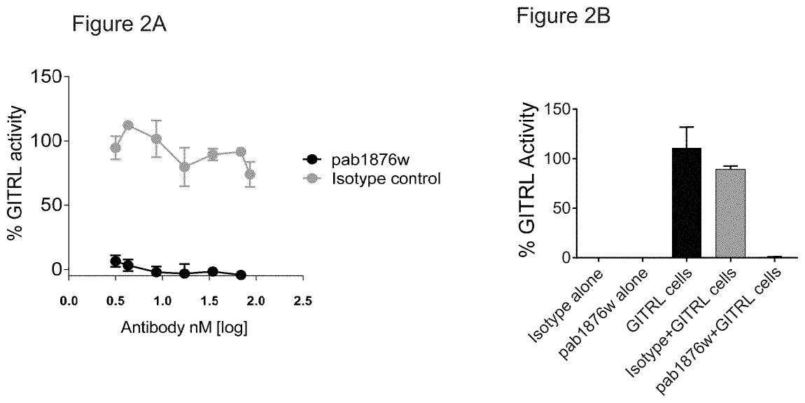

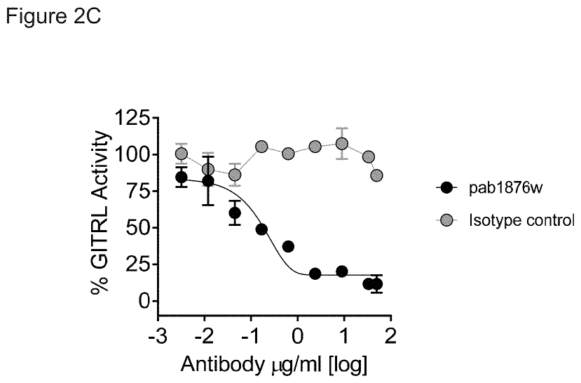

Incubation of soluble pab1876w with Jurkat-huGITR-NF-κB-luciferase reporter cells effectively blocked NF-X13-luciferase signaling triggered by GITRL-expressing cells (FIGS. 2A and 2B).

Further, the ability of pab1876w to block NF-κB signaling induced by cross-linked recombinant GITRL was examined. Briefly, Jurkat-huGITR-NF-κB-luciferase reporter cells were incubated with soluble pab1876w (50, 33, 8, 2.4, 0,6, 0.16, 0.04, 0.01, or 0.003 μg/ml) or an IgG₁ isotype control antibody in the presence of cross-linked GITRL (22 nM, HA tagged GITRL cross-linked with anti-HA). After 6 hours, the samples were equilibrated at room temperature and then an equal volume of room temperature Bio-Glo reagent (Promega) was added. Luminescence was read using an EnVision multilabel reader 2100.

As shown in FIG. 2C, soluble pab1876w reduced NF-κB-luciferase signaling in the reporter cells induced by cross-linked recombinant GITRL.

### 8.2 Example 2: Epitope Mapping of Anti-GITR Antibodies

This example characterizes the epitope of the following anti-GITR antibodies: a chimeric parental 231-32-15 antibody and its humanized versions (pab1876, pab1875, pab1967, pab1975, and pab1979). In addition, a reference anti-GITR antibody named m6C8 was also used in some studies for comparison. The antibody m6C8 was generated based on the variable regions of the antibody 6C8 provided in WO 06/105021 (herein incorporated by reference). The SEQ ID NOs corresponding to the heavy chain variable regions and light chain variable regions of these anti-GITR antibodies are listed in Table 6.

TABLE 6
VH and VL sequences of anti-GITR antibodies

| Antibody | VH (SEQ ID NO:) | VL (SEQ ID NO:) |
|---|---|---|
| 231-32-15 | 101 | 102 |
| pab1876 | 18 | 19 |
| pab1875 | 18 | 103 |
| pab1967 | 20 | 21 |
| pab1975 | 22 | 23 |
| pab1979 | 24 | 23 |
| m6C8 | 104 | 105 |

### 8.2.1 Epitope Competition—Cell Binding Assay

To confirm that the humanized variant antibodies retained the epitope specificity of the parental chimeric 231-32-15 antibody, a cell binding assay was performed, 1624-5 pre-B cells expressing the chimeric parental 231-32-15 antibody were harvested and 1×10⁶ cells were resuspended in 200 μl FACS buffer plus: i) biotinylated GITR (GITR-bio) (1:1000), preincubated for 15 min with 2 μg chimeric parental 231-32-15 antibody; ii) GITR-bio (1:1000), preincubated for 15 min with 2 μg; pab1875; iii) GITR-bio (1:1000), preincubated for 15 min with 2 μg pab1876; or iv) GITR-bio (1:1000). The cells were incubated for 20 min at 4° C. and then washed with 4 ml FACS buffer and centrifuged for 5 min at 300 g at 4° C. The cell pellet was resuspended in 200 μl FACS buffer plus streptavidin-PE (1:1000) and then incubated and washed as before. The cells were then resuspended in 200 μl FACS buffer for analysis using a FACS-Ariall (BD Biosciences).

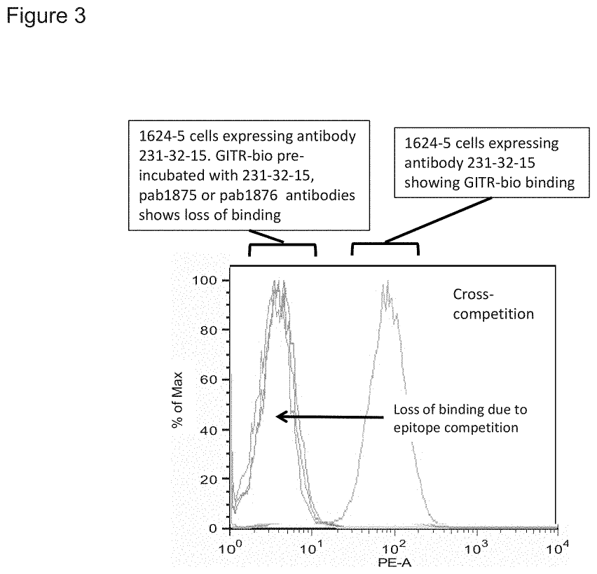

FIG. 3 shows that the humanized variant antibodies retained the epitope specificity of the chimeric parental 231-32-15 antibody. The right-hand profile shows the binding of GITR-bio to 1624-5 pre-B cells expressing the chimeric parental 231-32-25 antibody. However, when GITR-bio was pre-incubated with either chimeric parental 231-32-15, pab1875 or pab1876 antibodies, there was a loss of binding of GITR-bio to the 1624-5 cells (left-hand profile). The overlapping FACS profiles indicate that the humanized variants also show very similar GITR binding properties to each other and to the chimeric parental 231-32-15 antibody.

### 8.2.2 Epitope Competition Suspension Array Technology

Anti-GITR antibodies (25 μl) were diluted to 2 μg/ml in assay buffer (Roche 11112589001) and incubated with 1500 Luminex® beads (5 μl, Lurninex Corp, no 5 LC10005-01) coupled with anti-human IgG (F(ab)₂-specific, JIR, 105-006-097 overnight in 0.5 ml LoBind tubes (Eppendorf, 0030108.116) under shaking conditions, in the dark. This mixture was then transferred to pre-wetted 96-well filter plates (Millipore, MABVN1250). Plates were washed twice with 200 μl/well PBS to remove unbound antibody. At the same time 20 μg/ml of either the same anti-GITR antibodies, different anti-GITR antibodies, or assay buffer were incubated with 20 μl (1 μg/ml) R-PE labeled GITR antigen (R&D systems, di-sulfide-linked homodimer; 689-GR; in-house labeled with AbDSerotec LYNX Kit, LNK022RPE) for 1 hour in the dark at 650 rpm. The bead mixture and the antigen/antibody mixture were mixed 1:1 (20 μl from each) and incubated for one additional hour under shaking conditions (20° C., 650rpm), Directly before the measurement, 40 μl of assay buffer was added to each well and analysis was performed using a Luminex® 200 system (Millipore) and a readout of 100 beads in 48 μl sample volume. Binding was determined using the WI values of the non-competed control (100% binding, only assay buffer as competing compound).

When the chimeric parental 231-32-15 antibody was used as the captured antibody, full binding competition was observed with both humanized antibodies pab1875 and pab1876. When the anti-GITR antibody m6C8 was used as the captured antibody, no competition of binding was observed with the chimeric parental 231-32-15 antibody or the two humanized variants pab1875 and pab1876 (data not shown). These results indicate that m6C8 and the anti-GITR antibodies described herein recognize different epitopes on human GITR.

### 8.2.3 Epitope Competition Surface Plasmon Resonance

For epitope binning using surface plasmon resonance the “in tandem approach” was used (Abdiche, Y N et al., Analytical Biochemistry 386: 172-180 (2009)). For that purpose different chip surfaces were generated on a CM5 sensor chip (GE Healthcare, Series S CM5, BR-1005-30) using immobilization of different densities of GITR antigen (R&D systems, disulfide-linked homodimer; 689-GR). Flow cell 2 contained GITR antigen in low density (667 RU), medium density was assessed in flow cell 3 (1595 RU) and in flow cell 4, high density was achieved (4371 RU). In flow cell 1, ovalbumin (1289 RU, Pierce ThermoFisher 77120) was immobilized for reference. Immobilization was performed according to a standard protocol from the manufacturer (GE Healthcare) for amine coupling (activation of surface with 0.4 M EDC and 0.1 M NHS, GE Healthcare Amine coupling kit, BR-1000-50). Unreacted groups were inactivated with 1 M ethanol-amine-HCA, pH8.5. Afterwards anti-GITR antibodies were run through the different surfaces at a concentration of 300 nM (45 μg/ml) for 240 seconds at 5 μl/min. Using these conditions saturation of the GITR surface should have been reached. A dissociation time of 60 seconds was included before adding the competing antibody (300 nM, 5 μl/min). Regeneration of the chip surface was performed using 10 mM Glycine pH2.0 (GE Healthcare, BR-1003-55) for 60 seconds at 10 μl/min. Binning was performed using the response units (RU) of the non-competed control (100% binding, saturating conditions)

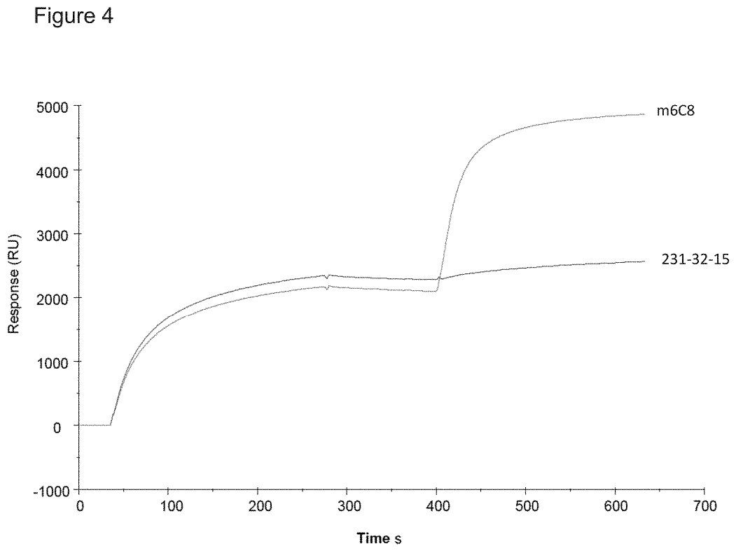

As is shown in FIG. 4, when the chimeric parental 231-32-15 antibody is first bound to GITR, no further binding of this antibody occurs. However, when the chimeric parental 231-32-15 antibody is first bound to GITR and the antibody m6C8 is applied, this antibody is still able to bind to GITR.

### 8.2.4 Epitope Mapping—PCR Mutagenesis and Alanine Scanning

In order to map the epitope on GITR to which anti-GITR antibodies described herein bind, error prone PCR was used to generate variants of the human GITR antigen. The variant GITR proteins were expressed on the surface of cells in a cellular library and these cells were screened for binding of the anti-GITR antibodies. As a positive control, a polyclonal anti-GITR antibody was used to confirm proper folding of the GITR protein. For variants of the human GITR antigen to which reduced or no antibody binding occurred, alanine scanning mutagenesis was performed to determine the precise epitope residues that were required for binding by the anti-GITR antibodies described herein.

### 8.2.4.1 Generation of Human GITR Variants

Error prone PCR mutagenesis was used to generate variants of human GITR with random mutations in the extracellular domain. For error prone PCR, the GeneMorphII Random Mutagenesis Kit (Stratagene) was used, according to the manufacturer's instructions. In brief, 20 PCR cycles in a volume of 50 μl was performed using an in-house construct as template (13 ng, construct number 4377 pMA-T-huGITR), 0.05 U/μl Mutazyme II DNA polymerase, 1× Mutazyme II reaction buffer, 0.2 μM of each primer and 0.2 mM of each deoxynucleoside-triphosphate (dATP, dCTP, dGTP, and dTTP). The samples were amplified by PCR (Eppendorf, Germany) using the following program: 95° C. for 2 min; 20 cycles of 95° C. for 30 sec, 56° C. for 30 sec, 72° C. for 1 min; and a final extension step of 72° C. for 10 min. The PCR product was gel purified using 1% agarose gel, the DNA band corresponding to the expected size of 720 bp was cut out and gel extraction was done using a NucleoSpin Gel and PCR cleanup kit from Macherey&Nagel according to the product manual. Purified DNA was ligated into an in-house expression vector via XhoI/EcoRI sites using T4 DNA ligase and a ratio of 1:3 (vector:insert), Ligation (25° C.) was stopped after 2 hours with a heat denaturation step for 10 min at 65° C. DNA from the ligation reaction was EtOH precipitated using yeast t-RNA. Standard digestion and ligation techniques were used. The ligation reaction was electroporated into DH10B cells (E. coli Electro Max DH10B electrocompetent cells, Invitrogen; 1900V/5ms). Electroporated bacteria were plated onto LB-agar 100 μg/ml ampicillin plates and approximately 1.9×10⁸ colonies were obtained.

All electroporated bacteria were then scratched from the plates and used for large-scale DNA plasmid preparation (Macherey&Nagel, NucleoBond Xtra Maxi Plus Kit), according to the manufacturer's instructions to generate a DNA library. A restriction enzyme digestion with XhoI/EcoRI and BsrGI/EcoRI was performed to quality control the library. Single clones were picked and sent for sequencing to determine the final library diversity.

### 8.2.4.2 Generation of a Cellular Library with Human GITR Variants

Standard techniques of transfection followed by transduction were used to express human GITR mutants on the surface of 1624-5 cells. For the generation of retroviral particles, a DNA library and vectors expressing retroviral proteins Gag, Pol and Env were transfected into a retroviral packaging cell line (HEK cells) using X-tremeGENE 9 DNA transfection reagent (Roche Diagnostics GmbH, Germany). The resulting retroviral particles accumulated in the cell culture supernatant of the retroviral packaging cells. Two days post transfection cell-free viral vector particle-containing supernatants were harvested and subjected to spin-infection of 1624-5 cells. A transduction efficiency (% human GITR expressing cells) of roughly 4% was obtained. Upon continuous culture for at least one additional day, cells were selected using puromycin (1.5 μg/ml). Untransduced cells served as negative controls (NC). After antibiotic selection, most cells stably expressed the human GITR antigen library on the cell surface. Non-viable cells were removed via a Ficoll separation step.

FACS was used to select cells expressing correctly folded human GITR mutants using a polyclonal anti-GITR antibody and to subsequently select individual cells expressing human GITR variants that did not bind to the anti-GITR chimeric parental 231-32-15 antibody. In brief, antibody binding cells were analyzed by FACS and cells that exhibited specific antibody binding were separated from the non-binding cell population by preparative, high-speed FACS (FACSAriaII, BD Biosciences). Antibody reactive or non-reactive cell pools were expanded again in tissue culture and, due to the stable expression phenotype of retrovirally transduced cells, cycles of antibody-directed cell sorting and tissue culture expansion were repeated, up to the point that a clearly detectable anti-GITR antibody (chimeric parental 231-32-15) non-reactive cell population was obtained. This anti-GITR antibody (chimeric parental 231-32-15) non-reactive cell population was subjected to a final, single-cell sorting step. After several days of cell expansion, single cell sorted cells were again tested for non-binding to anti-GITR chimeric parental 231-32-15 antibody and binding to a polyclonal anti-GITR antibody using 96 well plate analysis on a FACSCalibur (BD Biosciences).

### 8.2.4.3 Epitope Analysis

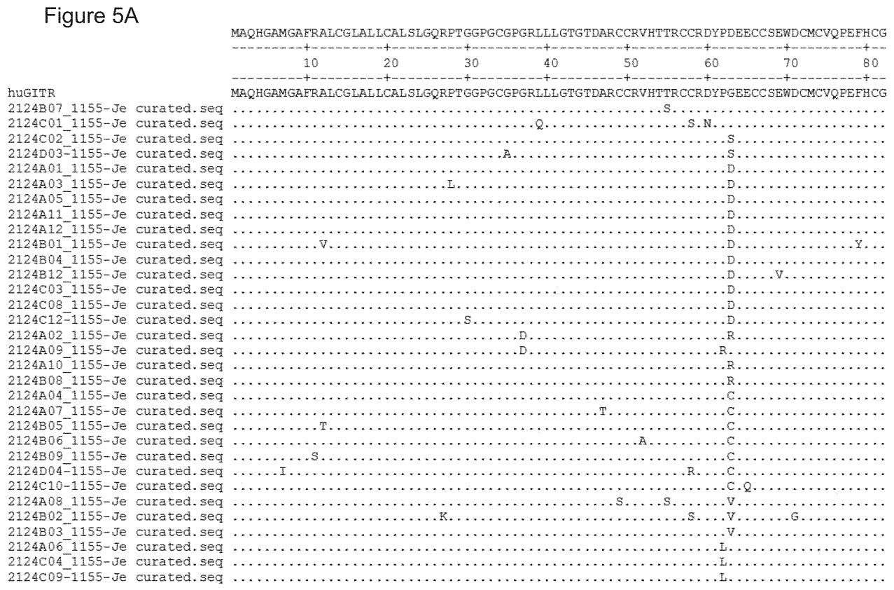

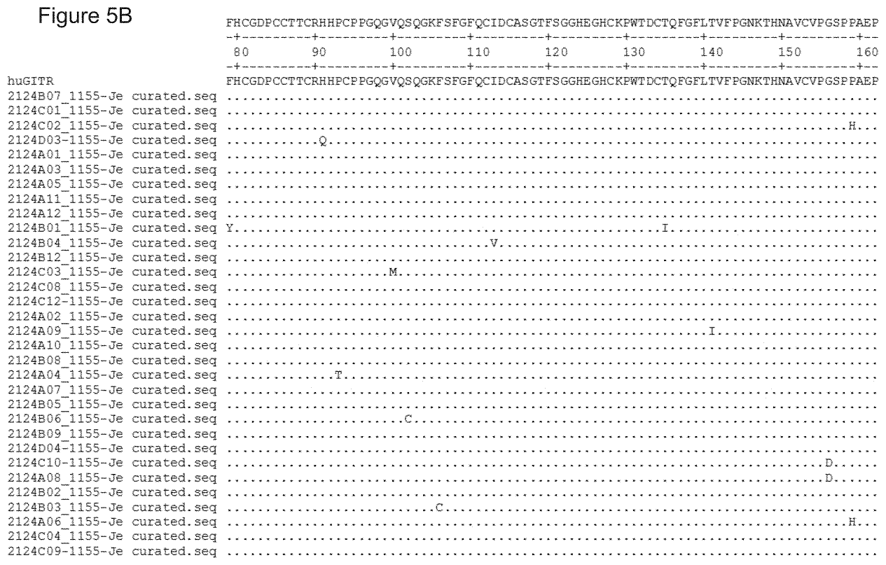

To connect phenotype (polyclonal anti-GITR+, chimeric parental 231-32-15-) with genotype, sequencing of single cell sorted huGITR variants was performed. FIGS. 5A and 5B show the alignment of sequences from these variants. The amino acid residues in FIGS. 5A and 5B are numbered according to the immature amino acid sequence of human GITR (SEQ ID NO: 41). Sequencing identified regions with increased mutations or “hot spots” (e.g., P62 and G63), providing an indication of the epitope on human GITR recognized by anti-GITR chimeric parental 231-32-15 antibody.

To confirm the precise amino acids of human GITR involved in binding to anti-GITR antibodies, alanine replacement of hot spot amino acids was performed. The following positions (numbered according to SEQ ID NO: 41) were separately mutated to an Alanine: P28A, T29A, G30A, G31A, P32A, T54A, T55A, R56A, C57A, C58A, R59A, D60A, Y61A, P62A, G63A, E64A, E65A, C66A, C67A, S68A, E69A, W70A, D71A, C72A, M73A, C74A, V75A, and Q76A. Standard techniques of transfection followed by transduction were used to express these human GITR alanine mutants on the surface of 1624-5 cells.

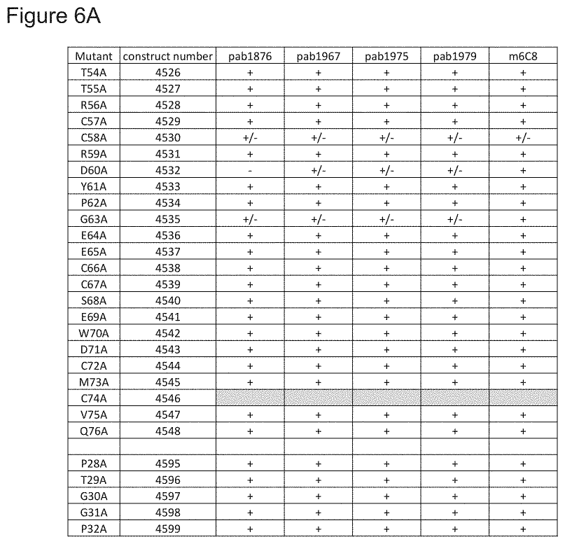

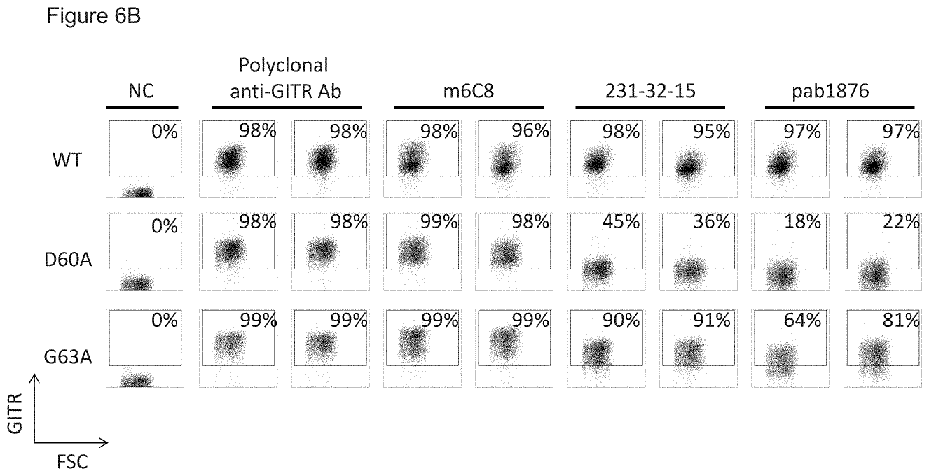

Finally, alanine mutants expressed on 1624-5 cells were tested in flow cytometry (FACSCalibur; BD Biosciences) for the binding of the anti-GITR humanized antibodies pab1876, pab1967, pab1975 and pab1979, and the reference antibody m6C8. Briefly, 1624-5 cells expressing individual human GITR alanine mutants were incubated with 2 μg/ml. of the monoclonal anti-GITR antibody pab1876, pab1967, pab1975, pab1979, or m6C8; or a polyclonal anti-GITR antibody (AF689, R&D systems) conjugated with APC, and Fe receptor block (1:200; BD Cat no. 553142) diluted in 100 μl FACS buffer (PBS+2% FCS) for 20 min at 4° C. After washing, the cells were incubated with a secondary anti-IgG antibody if necessary for detection (APC conjugated; BD Cat no. 109-136-097) diluted in 100 μl FACS buffer (PBS+2% FCS) for 20 min at 4° C. The cells were then washed and acquired using a flow cytometer (BD Biosciences), The mean fluorescence intensity (MFI) value of the tested monoclonal antibody was divided by the MFI value of the polyclonal antibody, generating an MFI ratio (monoclonal antibody/polyclonal antibody) for individual GITR alanine mutants. An average MFI ratio (“AMFI ratio”) was calculated based on the individual MFI ratios for all the mutants. FIG. 6A is a table summarizing the binding of pab1876, pat) 1967, pab1975, pab1979 and the reference antibody m6C8 to1624-5 cells expressing human GITR alanine mutants. An individual MFI ratio that is above 60% of the AMFI ratio is considered to indicate similar binding, after normalization, of that of the polyclonal antibody and is represented by “+” in FIG. 6A. An individual MFI ratio that is between 30% and 60% of the AMFI ratio is represented by “+/−” in FIG. 6A. An individual MFI ratio that is below 30% of the AMFI ratio is represented by “−” in FIG. 6A.

As shown in FIG. 6A, the D60A mutant and the G63A mutant, numbered according to SEQ ID NO: 41, specifically disrupted or weakened the binding of pab1876, pab1967, pab1975 and pab1979, but not that of the reference antibody m6C8. The C58A mutant disrupted the binding of all five antibodies and is likely a structural mutation rather than an epitope-specific one. The C74A mutant had weak expression and could not be used for binding comparison.

Furthermore, the anti-GITR antibodies 231-32-15, pab1876, and m6C8 were compared for their binding to wild type versus mutant human GITR. Briefly, wild type human GITR and two GITR alanine mutants (the D60A mutant and the G63A mutant, numbered according to SEQ ID NO: 41) were expressed on the surface of 1624-5 cells as described above and tested in a flow cytometry analysis as described above where cells were first stained using 2 μg/ml of the monoclonal antibodies 231-32-15, pab1876, and m6C8, or a polyclonal antibody conjugated to APC, and then stained using a secondary anti-IgG antibody if necessary for detection (APC conjugated; 1:1000; BD Cat No. 109-136-097). All the mean fluorescence intensity (MFI) values were calculated as the mean of two measurements. The MFI value of the tested monoclonal antibody for a particular cell type was divided by the MFI value of the polyclonal antibody for the same cell type, generating a total of nine MFI ratios (monoclonal antibodylpolyclonal antibody): MFI ratio_{231-32-15, WT}, MFI ratio_{pab1876, WT}, MFI ratio_{m6C8, WT}, MFI ratio_{231-32-15, D60A}, MFI ratio_{pab1876, D60A}, MFI ratio_{m6C8, D60A}, MFI ratio_{231-32-15, G63A}, MFI ratio_{pab1876, G631}, and MFI ratio_{m6C8, G63A}. The percentage of binding of an antibody to the GITR alanine mutants relative to the wild type GITR was calculated by dividing a particular MFI ratio for the GITR alanine mutants by the corresponding MFI ratio for the wild type (e.g., dividing MFI ratio_{pab1876, D60A} by MFI ratio_{pab1876, WT}). The percentage of reduction in binding was determined by calculating, e.g., 100%*(1-(MFI ratio_{pab1876, D60A}/MFI ratio_{pab1876, WT})).

As shown in FIG. 6B, the D60A mutant and the G63A mutant specifically disrupted or weakened the binding of 231-32-15 and pab1876, but not that of m6C8. The percentages shown in FIG. 6B are the percentages of GITR positive cells in each plot. When tested using the cells expressing GITR D60A, antibody binding was reduced by 82% and 88% for 231-32-15 and pab1876, respectively, compared with a 10% reduction for m6C8. Similarly, when tested using the cells expressing GITR G63A, the binding of 231-32-15 and pab1876 was reduced by 37% and 59%, respectively, whereas the binding of m6C8 was increased by 62%.

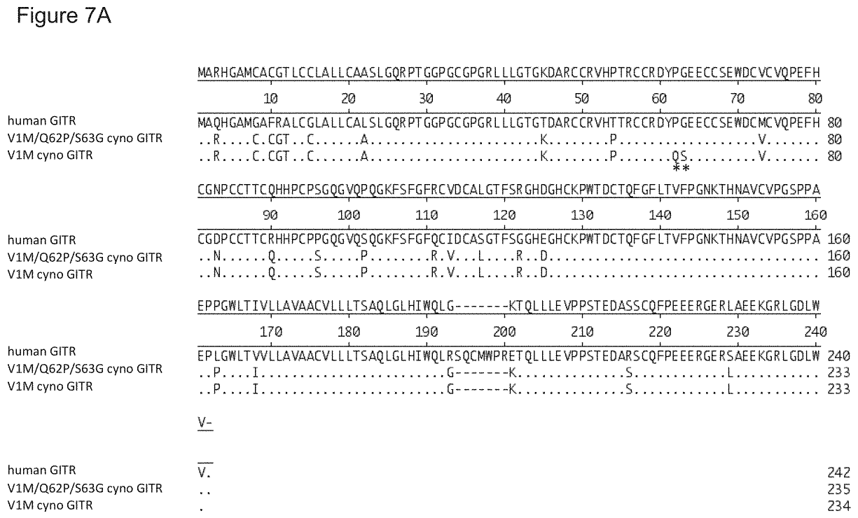

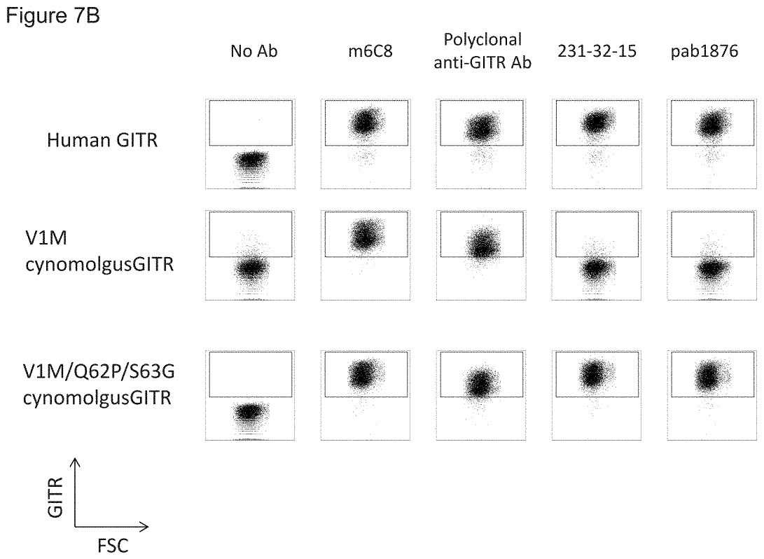

As further evidence for the binding characteristics of the anti-GITR antibodies, the binding of the antibodies to cynomolgus GITR was compared. The immature protein of cynomolgus GITR comprises the amino acid sequence of SEQ ID NO: 44. To increase protein expression, the first residue of the signal peptide of cynomolgus GITR was replaced by methionine, generating V1M cynomolgus GITR. A mutant cynomolgus GITR V1M/Q62P/S63G, where the amino acid residues at the positions 62 and 63 (GlnSer), numbered according to SEQ ID NO: 44, were replaced by the corresponding residues in human GITR (ProGly), was then generated. FIG. 7A is a sequence alignment between human GITR, V1M cynomolgus GITR, and V1M/Q62P/S63G cynomolgus GITR. The three proteins shown in FIG. 7A were expressed on the surface of 1624-5 cells as described above and tested in a flow cytometry analysis as described above where cells were first stained using 2 μg/ml of the monoclonal antibodies 231-32-15, pab1876, and m6C8, or a polyclonal antibody conjugated to APC, and then stained using a secondary anti-IgG antibody (APC conjugated; 1:1000; BD Cat no. 109-136-097).

As shown in FIG. 7B, the anti-GITR antibodies 231-32-15 and pab1876 displayed binding only to the cells expressing V1M/Q62P/S63G cynomolgus GITR, but not the cells expressing V1M cynomolgus GITR.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications, patents, or patent applications) cited herein are incorporated herein by reference in their entirety and for all purposes to the same extent as if each individual reference (e.g., publication, patent, or patent application) was specifically and individually indicated to be incorporated by reference in its entirety for all purposes.

Other embodiments are within the following claims.

1. An isolated antibody that specifically binds to human GITR, wherein the antibody comprises: (i) a first antigen-binding domain that specifically binds to human GITR; and (ii) a second antigen-binding domain that does not specifically bind to an antigen expressed by a human immune cell, wherein the first antigen-binding domain comprises:
(a) a heavy chain variable domain (VH) comprising a VH-complementarity determining region (CDR) 1 comprising the amino acid sequence of X₁YX₂MX₃ (SEQ ID NO:87), wherein X₁ is D, E or G; X₂ is A or V, and X₃ is Y or H; a VH-CDR2 comprising the amino acid sequence of X₁IX₂TX₃SGX₄X₅X₆YNQKFX₇X₈ (SEQ ID NO:88), wherein X₁ is V or L, X₂ is R, K or Q, X₃ is Y or F, X₄ is D, E or G, X₅ is V or L, X₆ is T or S, X₇ is K, R or Q, and X₈ is D, E or G; and a VH-CDR3 comprising the amino acid sequence of SEQ ID NO:3; and
(b) a light chain variable domain (VL) comprising a VL-CDR1 comprising the amino acid sequence of KSSQSLLNSX₁NQKNYLX₂ (SEQ ID NO:90), wherein X₁ is G or S, and X₂ is T or S; a VL-CDR2 comprising the amino acid sequence of SEQ ID NO:5; and a VL-CDR3 comprising the amino acid sequence of QNX₁YSX₂PYT (SEQ ID NO:92), wherein X₁ is D or E; and X₂ is Y, F or S.

2. (canceled)

3. The antibody of claim 1, wherein the first antigen-binding domain:
(a) binds to the same epitope of human GITR as an antibody comprising a VH comprising the amino acid sequence of SEQ ID NO:18 and a VL comprising the amino acid sequence of SEQ ID NO:19;
(b) exhibits, as compared to binding to a human GITR sequence of residues 26 to 241 of SEQ ID NO:41, reduced or absent binding to a protein identical to residues 26 to 241 of SEQ ID NO:41 except for the presence of a D60A or G63A amino acid substitution, numbered according to SEQ ID NO:41;
(c) specifically binds to an epitope of GITR comprising at least one amino acid in residues 60-63 of SEQ ID NO:41; or
(d) specifically binds to each of i) human GITR, comprising amino acid residues 26 to 241 of SEQ ID NO:41; and ii) a variant of cynomolgus GITR, said variant comprising amino acid residues 26-234 of SEQ ID NO:46, wherein the antigen-binding domain that specifically binds to human GITR does not specifically bind to cynomolgus GITR comprising amino acid residues 26-234 of SEQ ID NO:44.

4. (canceled)

5. The antibody of claim 1, wherein the first antigen-binding domain comprises:
(a) CDRs comprising the amino acid sequences of SEQ ID NOs: 1-6;
(b) a VH-CDR1 comprising an amino acid sequence selected from the group consisting of SEQ ID NOs: 7-9;
(c) a VH-CDR2 comprising an amino acid sequence selected from the group consisting of SEQ ID NOs: 10-13;
(d) a VL-CDR1 comprising the amino acid sequence of SEQ ID NO: 14 or 15;
(e) a VL-CDR3 comprising the amino acid sequence of SEQ ID NO: 16 or 17;
(f) VH-CDR1, VH-CDR2, and VH-CDR3 sequences set forth in SEQ ID NOs: 7, 10, and 3; SEQ ID NOs: 8, 11, and 3; SEQ ID NOs: 9, 12, and 3; or SEQ ID NOs: 9, 13, and 3, respectively; and/or VL-CDR1, VL-CDR2, and VL-CDR3 sequences set forth in SEQ ID NOs: 14, 5, and 16; or SEQ ID NOs: 15, 5, and 17, respectively; or
(g) VH-CDR1, VH-CDR2, VH-CDR3, VL-CDR1, VL-CDR2, and VL-CDR3 sequences set forth in SEQ ID NOs: 7, 10, 3, 14, 5, and 16, respectively.

6. The antibody of claim 1, wherein the first antigen-binding domain comprises:
(a) a VH and a VL, wherein the VH comprises an amino acid sequence selected from the group consisting of SEQ ID NOs: 18, 20, 22, 24, and 25, and/or the VL comprises an amino acid sequence selected from the group consisting of SEQ ID NOs: 19, 21, 23, and 26;
(b) a VH comprising the sequence set forth in SEQ ID NO:25;
(c) a VH comprising an amino acid sequence at least 75%, 80%, 85%, 90%, 95%, or 99% identical to an amino acid sequence selected from the group consisting of SEQ ID NOs: 18, 20, 22, and 24;
(d) a VH comprising an amino acid sequence selected from the group consisting of SEQ ID NOs: 18, 20, 22, and 24;
(e) a VH comprising an amino acid sequence derived from a human IGHV1-2 germline sequence;
(f) a VL comprising the amino acid sequence of SEQ ID NO: 26;
(g) a VL comprising an amino acid sequence at least 75%, 80%, 85%, 90%, 95%, or 99% identical to an amino acid sequence selected from the group consisting of SEQ ID NOs: 19, 21, and 23;
(h) a VL comprising an amino acid sequence selected from the group consisting of SEQ ID NOs: 19, 21, and 23;
(i) a VL comprising an amino acid sequence derived from a human IGKV4-1 germline sequence; or
(j) VH and VL sequences set forth in SEQ ID NOs: 18 and 19, SEQ ID NOs: 20 and 21, SEQ ID NOs: 22 and 23, or SEQ ID NOs: 24 and 23, respectively.

7. (canceled)

8. The antibody of claim 1, wherein the second antigen-binding domain specifically binds to a non-human antigen, a viral antigen, an HIV antigen, or hen egg lysozyme.

9-31. (canceled)

32. The antibody of claim 1, wherein the first antigen-binding domain comprises:
(a) a heavy chain comprising the amino acid sequence of SEQ ID NOs: 29, 30, 36, 74 75, or 81; and/or
(b) a light chain comprising the amino acid sequence of SEQ ID NO: 37 or 38.

33-45. (canceled)

46. The antibody of any one of claim 1, wherein:
(a) the first antigen-binding domain comprises a first human IgG₁ heavy chain and the second antigen-binding domain comprises a second human IgG₁ heavy chain, and wherein the first and second heavy chains comprise an identical mutation selected from the group consisting of N297A, N297Q, D265A, and a combination thereof, numbered according to the EU numbering system,
(b) the first antigen-binding domain comprises a first human IgG2 heavy chain and the second antigen-binding domain comprises a second human IgG2 heavy chain, and wherein the first and second heavy chains comprise a C127S mutation, numbered according to Kabat; or
(c) the first antigen-binding domain comprises a first human IgG4 heavy chain and the second antigen-binding domain comprises a second human IgG4 heavy chain, and wherein the first and second heavy chains comprise a S228P mutation, numbered according to the EU numbering system.

47-51. (canceled)

52. The antibody of claim 1, wherein the antibody:
(a) is antagonistic to human GITR;
(b) deactivates, reduces, or inhibits an activity of human GITR;
(c) inhibits or reduces binding of human GITR to human GITR ligand;
(d) inhibits or reduces human GITR signaling;
(e) inhibits or reduces human GITR signaling induced by human GITR ligand;
(f) decreases CD4+ T cell proliferation induced by synovial fluid from rheumatoid arthritis patients;
(g) increases survival of NOG mice transplanted with human PBMCs; and/or
(h) increases proliferation of regulatory T cells in a GVHD model.

53-60. (canceled)

61. A pharmaceutical composition comprising the antibody of claim 1, and a pharmaceutically acceptable excipient.

62. A method of modulating an immune response in a subject, the method comprising administering to the subject an effective amount of the antibody of claim 1.

63. (canceled)

64. A method of treating an autoimmune or inflammatory disease or disorder in a subject, the method comprising administering to the subject an effective amount of the antibody of claim 1.

65. (canceled)

66. A method of treating an infectious disease in a subject, the method comprising administering to the subject an effective amount of the antibody of claim 1.

67. (canceled)

68. A method for detecting GITR in a sample comprising contacting the sample with the antibody of claim 1.

69. A kit comprising the antibody of claim 1, and a) a detection reagent, b) a GITR antigen, c) a notice that reflects approval for use or sale for human administration, or d) a combination thereof.

70-84. (Canceled)


SEQUENCE LISTING (133 sequences)

SEQ ID NO: 1 - protein, 5 aa, Artificial sequence
  GITR HCDR1 consensus
  MISC_FEATURE (1)..(1): Xaa is Asp, Gly or Glu
  MISC_FEATURE (5)..(5): Xaa is Tyr or His
XYAMX

SEQ ID NO: 2 - protein, 17 aa, Artificial sequence
  GITR HCDR2 consensus
  MISC_FEATURE (1)..(1): Xaa is Val or Leu
  MISC_FEATURE (8)..(8): Xaa is Asp or Gly
  MISC_FEATURE (10)..(10): Xaa is Thr or Ser
  MISC_FEATURE (16)..(16): Xaa is Lys, Arg or Gln
  MISC_FEATURE (17)..(17): Xaa is Asp, Glu or Gly
XIRTYSGXVXYNQKFXX

SEQ ID NO: 3 - protein, 9 aa, Artificial sequence
  GITR HCDR3 consensus (pab1876/pab1967/pab1975/pab1979 HCDR3)
SGTVRGFAY

SEQ ID NO: 4 - protein, 17 aa, Artificial sequence
  GITR LCDR1 consensus
  MISC_FEATURE (10)..(10): Xaa is Gly or Ser
KSSQSLLNSXNQKNYLT

SEQ ID NO: 5 - protein, 7 aa, Artificial sequence
  GITR LCDR2 consensus (pab1876/pab1967/pab1975/pab1979 LCDR2)
WASTRES

SEQ ID NO: 6 - protein, 9 aa, Artificial sequence
  GITR LCDR3 consensus
  MISC_FEATURE (3)..(3): Xaa is Asp or Glu
  MISC_FEATURE (6)..(6): Xaa is Tyr or Phe
QNXYSXPYT

SEQ ID NO: 7 - protein, 5 aa, Artificial sequence
  pab1876 HCDR1
DYAMY

SEQ ID NO: 8 - protein, 5 aa, Artificial sequence
  pab1967 HCDR1
GYAMH

SEQ ID NO: 9 - protein, 5 aa, Artificial sequence
  pab1975/pab1979 HCDR1
EYAMH

SEQ ID NO: 10 - protein, 17 aa, Artificial sequence
  pab1876 HCDR2
VIRTYSGDVTYNQKFKD

SEQ ID NO: 11 - protein, 17 aa, Artificial sequence
  pab1967 HCDR2
LIRTYSGGVSYNQKFRE

SEQ ID NO: 12 - protein, 17 aa, Artificial sequence
  pab1975 HCDR2
LIRTYSGGVSYNQKFQG

SEQ ID NO: 13 - protein, 17 aa, Artificial sequence
  pab1979 HCDR2
VIRTYSGGVSYNQKFQE

SEQ ID NO: 14 - protein, 17 aa, Artificial sequence
  pab1876/pab1975/pab1979 LCDR1
KSSQSLLNSGNQKNYLT

SEQ ID NO: 15 - protein, 17 aa, Artificial sequence
  pab1967 LCDR1
KSSQSLLNSSNQKNYLT

SEQ ID NO: 16 - protein, 9 aa, Artificial sequence
  pab1876/pab1975/pab1979 LCDR3
QNDYSYPYT

SEQ ID NO: 17 - protein, 9 aa, Artificial sequence
  pab1967 LCDR3
QNEYSFPYT

SEQ ID NO: 18 - protein, 118 aa, Artificial sequence
  pab1876 VH
QVQLVQSGAEVKKPGASVKVSCKGSGYTFTDYAMYWVRQAPGQGLEWIGVIRTYSGDVTYNQKFKDRATMTVDKSISTAYMELSRLRSDDTAVYYCAKSGTVRGFAYWGQGTLVTVSS

SEQ ID NO: 19 - protein, 113 aa, Artificial sequence
  pab1876 VL
DIVMTQSPDSLAVSLGERATINCKSSQSLLNSGNQKNYLTWYQQKPGQPPKLLIYWASTRESGVPDRFSGSGSGTDFTLTISSLQAEDVAVYHCQNDYSYPYTFGQGTKLEIK

SEQ ID NO: 20 - protein, 118 aa, Artificial sequence
  pab1967 VH
QVQLVQSGAEVKKPGASVKVSCKGSGYTFTGYAMHWVRQAPGQGLEWMGLIRTYSGGVSYNQKFRERATMTVDTSISTAYMELSRLRSDDTAVYYCAKSGTVRGFAYWGQGTLITVSS

SEQ ID NO: 21 - protein, 113 aa, Artificial sequence
  pab1967 VL
DIVMTQSPDSLAVSLGERATINCKSSQSLLNSSNQKNYLTWYQQKPGQPPKLLIYWASTRESGVPDRFTGSGSGTDFTLTISSVQAEDVAVYHCQNEYSFPYTFGQGTKLEIK

SEQ ID NO: 22 - protein, 118 aa, Artificial sequence
  pab1975 VH
QVQLVQSGAEVKKPGASVKVSCKASGYTFTEYAMHWVRQAPGQGLEWMGLIRTYSGGVSYNQKFQGRATMTVDTSISTAYMELSRLRSDDTAVYYCAKSGTVRGFAYWGQGTLVTVSS

SEQ ID NO: 23 - protein, 113 aa, Artificial sequence
  pab1975/pab1979 VL
DIVMTQSPDSLAVSLGERATINCKSSQSLLNSGNQKNYLTWYQQKPGQPPKLLIYWASTRESGVPDRFSGSGSGTDFTLTISSLQAEDVAVYYCQNDYSYPYTFGQGTKLEIK

SEQ ID NO: 24 - protein, 118 aa, Artificial sequence
  pab1979 VH
QVQLVQSGAEVKKPGASVKVSCKASGYTFTEYAMHWVRQAPGQGLEWMGVIRTYSGGVSYNQKFQERVTMTVDTSISTAYMELSRLRSDDTAVYYCAKSGTVRGFAYWGQGTLVTVSS

SEQ ID NO: 25 - protein, 118 aa, Artificial sequence
  GITR VH consensus
  MISC_FEATURE (24)..(24): Xaa is Gly or Ala
  MISC_FEATURE (31)..(31): Xaa is Asp, Gly or Glu
  MISC_FEATURE (35)..(35): Xaa is Tyr or His
  MISC_FEATURE (48)..(48): Xaa is Ile or Met
  MISC_FEATURE (50)..(50): Xaa is Val or Leu
  MISC_FEATURE (57)..(57): Xaa is Asp or Gly
  MISC_FEATURE (59)..(59): Xaa is Thr or Ser
  MISC_FEATURE (65)..(65): Xaa is Lys, Arg or Gln
  MISC_FEATURE (66)..(66): Xaa is Asp, Glu or Gly
  MISC_FEATURE (68)..(68): Xaa is Ala or Val
  MISC_FEATURE (74)..(74): Xaa is Lys or Thr
  MISC_FEATURE (114)..(114): Xaa is Val or Ile
QVQLVQSGAEVKKPGASVKVSCKXSGYTFTXYAMXWVRQAPGQGLEWXGXIRTYSGXVXYNQKFXXRXTMTVDXSISTAYMELSRLRSDDTAVYYCAKSGTVRGFAYWGQGTLXTVSS

SEQ ID NO: 26 - protein, 113 aa, Artificial sequence
  GITR VL consensus
  MISC_FEATURE (33)..(33): Xaa is Gly or Ser
  MISC_FEATURE (69)..(69): Xaa is Ser or Thr
  MISC_FEATURE (84)..(84): Xaa is Leu or Val
  MISC_FEATURE (93)..(93): Xaa is His or Tyr
  MISC_FEATURE (97)..(97): Xaa is Asp or Glu
  MISC_FEATURE (100)..(100): Xaa is Tyr or Phe
DIVMTQSPDSLAVSLGERATINCKSSQSLLNSXNQKNYLTWYQQKPGQPPKLLIYWASTRESGVPDRFXGSGSGTDFTLTISSXQAEDVAVYXCQNXYSXPYTFGQGTKLEIK

SEQ ID NO: 27 - protein, 98 aa, Artificial sequence
  GITR VH germline IGHV1-2*02
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAR

SEQ ID NO: 28 - protein, 101 aa, Artificial sequence
  GITR VL germline IGKV4-1*01
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTP

SEQ ID NO: 29 - protein, 448 aa, Artificial sequence
  pab1876 HC IgG1
QVQLVQSGAEVKKPGASVKVSCKGSGYTFTDYAMYWVRQAPGQGLEWIGVIRTYSGDVTYNQKFKDRATMTVDKSISTAYMELSRLRSDDTAVYYCAKSGTVRGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 448 aa, Artificial sequence
  pab1876 HC IgG1 N297A
QVQLVQSGAEVKKPGASVKVSCKGSGYTFTDYAMYWVRQAPGQGLEWIGVIRTYSGDVTYNQKFKDRATMTVDKSISTAYMELSRLRSDDTAVYYCAKSGTVRGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31-35 - skipped (5 entries)

SEQ ID NO: 36 - protein, 445 aa, Artificial sequence
  pab1876 HC IgG4 S228P
QVQLVQSGAEVKKPGASVKVSCKGSGYTFTDYAMYWVRQAPGQGLEWIGVIRTYSGDVTYNQKFKDRATMTVDKSISTAYMELSRLRSDDTAVYYCAKSGTVRGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - protein, 220 aa, Artificial sequence
  pab1876w LC
DIVMTQSPDSLAVSLGERATINCKSSQSLLNSGNQKNYLTWYQQKPGQPPKLLIYWASTRESGVPDRFSGSGSGTDFTLTISSLQAEDVAVYHCQNDYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 220 aa, Artificial sequence
  pab1876 LC T109S
DIVMTQSPDSLAVSLGERATINCKSSQSLLNSGNQKNYLTWYQQKPGQPPKLLIYWASTRESGVPDRFSGSGSGTDFTLTISSLQAEDVAVYHCQNDYSYPYTFGQGTKLEIKRSVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - skipped

SEQ ID NO: 40 - protein, 216 aa, Homo sapiens
QRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEPLGWLTVVLLAVAACVLLLTSAQLGLHIWQLRSQCMWPRETQLLLEVPPSTEDARSCQFPEEERGERSAEEKGRLGDLWV

SEQ ID NO: 41 - protein, 241 aa, Homo sapiens
MAQHGAMGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEPLGWLTVVLLAVAACVLLLTSAQLGLHIWQLRSQCMWPRETQLLLEVPPSTEDARSCQFPEEERGERSAEEKGRLGDLWV

SEQ ID NO: 42 - protein, 255 aa, Homo sapiens
MAQHGAMGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCCWRCRRRPKTPEAASSPRKSGASDRQRRRGGWETCGCEPGRPPGPPTAASPSPGAPQAAGALRSALGRALLPWQQKWVQEGGSDQRPGPCSSAAAAGPCRRERETQSWPPSSLAGPDGVGS

SEQ ID NO: 43 - protein, 234 aa, Homo sapiens
MAQHGAMGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEPLGWLTVVLLAVAACVLLLTSAQLGLHIWQLRKTQLLLEVPPSTEDARSCQFPEEERGERSAEEKGRLGDLWV

SEQ ID NO: 44 - protein, 234 aa, Cynomolgus monkey
VARHGAMCACGTLCCLALLCAASLGQRPTGGPGCGPGRLLLGTGKDARCCRVHPTRCCRDYQSEECCSEWDCVCVQPEFHCGNPCCTTCQHHPCPSGQGVQPQGKFSFGFRCVDCALGTFSRGHDGHCKPWTDCTQFGFLTVFPGNKTHNAVCVPGSPPAEPPGWLTIVLLAVAACVLLLTSAQLGLHIWQLGKTQLLLEVPPSTEDASSCQFPEEERGERLAEEKGRLGDLWV

SEQ ID NO: 45 - protein, 234 aa, Cynomolgus monkey
MARHGAMCACGTLCCLALLCAASLGQRPTGGPGCGPGRLLLGTGKDARCCRVHPTRCCRDYQSEECCSEWDCVCVQPEFHCGNPCCTTCQHHPCPSGQGVQPQGKFSFGFRCVDCALGTFSRGHDGHCKPWTDCTQFGFLTVFPGNKTHNAVCVPGSPPAEPPGWLTIVLLAVAACVLLLTSAQLGLHIWQLGKTQLLLEVPPSTEDASSCQFPEEERGERLAEEKGRLGDLWV

SEQ ID NO: 46 - protein, 234 aa, Artificial sequence
  V1M/Q62P/S63G cynomolgus GITR immature protein
MARHGAMCACGTLCCLALLCAASLGQRPTGGPGCGPGRLLLGTGKDARCCRVHPTRCCRDYPGEECCSEWDCVCVQPEFHCGNPCCTTCQHHPCPSGQGVQPQGKFSFGFRCVDCALGTFSRGHDGHCKPWTDCTQFGFLTVFPGNKTHNAVCVPGSPPAEPPGWLTIVLLAVAACVLLLTSAQLGLHIWQLGKTQLLLEVPPSTEDASSCQFPEEERGERLAEEKGRLGDLWV

SEQ ID NO: 47-73 - skipped (27 entries)

SEQ ID NO: 74 - protein, 447 aa, Artificial Sequence
  pab1876 HC IgG1
QVQLVQSGAEVKKPGASVKVSCKGSGYTFTDYAMYWVRQAPGQGLEWIGVIRTYSGDVTYNQKFKDRATMTVDKSISTAYMELSRLRSDDTAVYYCAKSGTVRGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 75 - protein, 447 aa, Artificial Sequence
  pab1876 HC IgG1 N297A
QVQLVQSGAEVKKPGASVKVSCKGSGYTFTDYAMYWVRQAPGQGLEWIGVIRTYSGDVTYNQKFKDRATMTVDKSISTAYMELSRLRSDDTAVYYCAKSGTVRGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 76-80 - skipped (5 entries)

SEQ ID NO: 81 - protein, 444 aa, Artificial Sequence
  pab1876 HC IgG4 S228P
QVQLVQSGAEVKKPGASVKVSCKGSGYTFTDYAMYWVRQAPGQGLEWIGVIRTYSGDVTYNQKFKDRATMTVDKSISTAYMELSRLRSDDTAVYYCAKSGTVRGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 82-86 - skipped (5 entries)

SEQ ID NO: 87 - protein, 5 aa, Artificial Sequence
  H-CDR1 anti-GITR consensus
  MISC_FEATURE (1)..(1): Xaa is Asp, Glu or Gly
  MISC_FEATURE (3)..(3): Xaa is Ala or Val
  MISC_FEATURE (5)..(5): Xaa is Tyr or His
XYXMX

SEQ ID NO: 88 - protein, 17 aa, Artificial Sequence
  H-CDR2 anti-GITR consensus
  MISC_FEATURE (1)..(1): Xaa is Val or Leu
  MISC_FEATURE (3)..(3): Xaa is Arg, Lys or Gln
  MISC_FEATURE (5)..(5): Xaa is Tyr or Phe
  MISC_FEATURE (8)..(8): Xaa is Asp, Glu or Gly
  MISC_FEATURE (9)..(9): Xaa is Val or Leu
  MISC_FEATURE (10)..(10): Xaa is Thr or Ser
  MISC_FEATURE (16)..(16): Xaa is Lys, Arg or Gln
  MISC_FEATURE (17)..(17): Xaa is Asp, Glu or Gly
XIXTXSGXXXYNQKFXX

SEQ ID NO: 89 - skipped

SEQ ID NO: 90 - protein, 17 aa, Artificial Sequence
  L-CDR1 anti-GITR consensus
  MISC_FEATURE (10)..(10): Xaa is Gly or Ser
  MISC_FEATURE (17)..(17): Xaa is Thr or Ser
KSSQSLLNSXNQKNYLX

SEQ ID NO: 91 - skipped

SEQ ID NO: 92 - protein, 9 aa, Artificial Sequence
  L-CDR3 anti-GITR consensus
  MISC_FEATURE (3)..(3): Xaa is Asp or Glu
  MISC_FEATURE (6)..(6): Xaa is Tyr, Phe or Ser
QNXYSXPYT

SEQ ID NO: 93 - protein, 330 aa, Artificial Sequence
  Human IgG1 constant region consensus sequence
  MISC_FEATURE (97)..(97): Xaa is Lys or Arg
  MISC_FEATURE (239)..(239): Xaa is Asp or Glu
  MISC_FEATURE (241)..(241): Xaa is Leu or Met
  MISC_FEATURE (314)..(314): Xaa is Gly or Ala
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEXLHNHYTQKSLSLSPGK

SEQ ID NO: 94 - protein, 330 aa, Artificial Sequence
  Human IgG1 G1m3 allotype
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 330 aa, Artificial Sequence [sequence omitted]

SEQ ID NO: 96 - protein, 330 aa, Artificial Sequence
  Human IgG1, G1m17,1,2 allotype
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 97 - protein, 327 aa, Artificial Sequence
  Human IgG4 wild type constant regions
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLSSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 98 - protein, 327 aa, Artificial Sequence
  IgG4 constant regions S228P modification
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 99 - protein, 326 aa, Artificial Sequence
  Human IgG2 wild type constant region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 100 - protein, 326 aa, Artificial Sequence
  IgG2 constant region C127S modification
ASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 101 - protein, 118 aa, Artificial Sequence
  231-32-15 VH
QVQLLQSGTELVRPGVSVKISCKGSGYTFTDYAMYWVKQSHAKSLEWIGVIRTYSGDVTYNQKFKDKATMTVDKSSSIAYMELARLSSEDSAIYYCAKSGTVRGFAYWGQGTLVTVSS

SEQ ID NO: 102 - protein, 113 aa, Artificial Sequence
  231-32-15 VL
DIVMTQSPSSLTVTAGEKVIMSCKSSQSLLNSGNQKNYLTWYQQKPGQPPKLLIYWASTRESGVPDRFTGSGSGTDFTLTISSVQAEDLAVYHCQNDYSYPYTFGGGTKLEIK

SEQ ID NO: 103 - protein, 113 aa, Artificial Sequence
  pab1875 VL
DIVMTQSPPTLSLSPGERVTLSCKSSQSLLNSGNQKNYLTWYQQKPGQAPRLLIYWASTRESGIPARFSGSGSGTDFTLTISSLQPEDFAVYHCQNDYSYPYTFGQGTKLEIK

SEQ ID NO: 104 - protein, 119 aa, Artificial Sequence
  m6C8 VH
QVTLKESGPGILKPSQTLSLTCSFSGFSLSTSGMGVGWIRQPSGKGLEWLAHIWWDDDKYYNPSLKSQLTISKDTSRNQVFLKITSVDTADAATYYCARTRRYFPFAYWGQGTLVTVSS

SEQ ID NO: 105 - protein, 107 aa, Artificial Sequence
  m6C8 VL
DIVMTQSQKFMSTSVGDRVSVTCKASQNVGTNVAWYQQKPGQSPKALIYSASYRYSGVPDRFTGSGSGTDFTLTINNVHSEDLAEYFCQQYNTDPLTFGAGTKLEIK

SEQ ID NO: 106-125 - skipped (20 entries)

SEQ ID NO: 126 - protein, 329 aa, Artificial Sequence
  Human IgG1 constant region consensus sequence
  MISC_FEATURE (97)..(97): Xaa can be Lys or Arg
  MISC_FEATURE (239)..(239): Xaa can be Asp or Glu
  MISC_FEATURE (241)..(241): Xaa can be Leu or Met
  MISC_FEATURE (314)..(314): Xaa can be Gly or Ala
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEXLHNHYTQKSLSLSPG

SEQ ID NO: 127 - protein, 329 aa, Artificial Sequence
  Human IgG1 G1m3 allotype
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 128 - protein, 329 aa, Artificial Sequence
  Human IgG1 G1m17,1 allotype
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 129 - protein, 329 aa, Artificial Sequence
  Human IgG1, G1m17,1,2 allotype
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPG

SEQ ID NO: 130 - protein, 326 aa, Artificial Sequence
  Human IgG4 wild type constant regions
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLSSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 131 - protein, 326 aa, Artificial Sequence
  IgG4 constant regions S228P modification
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 132 - protein, 325 aa, Artificial Sequence
  Human IgG2 wild type constant region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 133 - protein, 325 aa, Artificial Sequence
  IgG2 constant region C127S modification
ASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG